(19) United States
(12) Patent Application Publication
(10) Pub. No.: US 2020/0362303 A1
(43) Pub. Date: Nov. 19, 2020

(54) MAINTENANCE-AND-AMPLIFICATION METHOD AND DIFFERENTIATION INDUCTION METHOD FOR PRIMORDIAL GERM CELLS/PRIMORDIAL GERM CELL-LIKE CELLS

(71) Applicant: KYOTO UNIVERSITY, Kyoto-shi, Kyoto (JP)

(72) Inventors: Mitinori SAITOU, Kyoto (JP); Hiroshi OHTA, Kyoto (JP); Hidetaka MIYAUHI, Kyoto (JP)

(73) Assignee: KYOTO UNIVERSITY, Kyoto-shi, Kyoto (JP)

(21) Appl. No.: 16/768,462

(22) PCT Filed: Nov. 30, 2018
(86) PCT No.: PCT/JP2018/045011

§ 371 Date: May 29, 2020

(30) Foreign Application Priority Data
Nov. 30, 2017 (JP) 2017-231294

(51) Int. Cl. C12N 5/0735; C12N 5/075

(52) U.S. Cl. CPC C12N 5/0611; C12N 5/0609

(57) ABSTRACT

The present invention provides a method for expanding PGC/PGCLC, including culturing PGC/PGCLC in the presence of a phosphodiesterase 4 (PDE4) inhibitor and/or cyclosporine A, further in the presence of forskolin, and a method for inducing oocytes from PGC/PGCLC, including culturing PGC/PGCLC in the presence of bone forming protein (BMP) and retinoic acid (RA).

### TECHNICAL FIELD

The present invention relates to a method for expanding a primordial germ cell or a primordial germ cell-like cell, a method for inducing oogenesis from the cell, a reagent therefor, and the like.

### BACKGROUND ART

A major problem in developmental biology is the reconstitution of essential developmental pathways in vitro. This not only provides new experimental opportunities but also serves as the basis for medical applications. The present inventors previously established a culture system for inducing embryonic stem cells (sometimes to be abbreviated as “ES cell”, “ESC”)/induced pluripotent stem cells (sometimes to be abbreviated as “iPS cell”, “iPSC”) into epiblast like-cells (sometimes to be abbreviated as “EpiLC”) by using cytokine containing activin A and basic fibroblast growth factor (bFGF), and thereafter inducing into primordial germ cell (sometimes to be abbreviated as “PGC”)-like cells (sometimes to be abbreviated as “PGC-like cell”, “PGCLC”) by using cytokine containing BMP4 (see, for example, patent document 1, non-patent document 1). Furthermore, they succeeded in transplanting the PGCLC under the egg sac of newborn mice, differentiating same into ovum, and obtaining normal offspring therefrom (non-patent document 2). In addition, they also succeeded in inducing ovum from pluripotent stem cells (sometimes to be abbreviated as “PSC”) in vitro via PGCLC (non-patent document 3).

However, it is difficult to control proliferation and differentiation of PGC/PGCLC in vitro, which poses a problem in advancing research.

It has been reported that Forskolin is effective for PGC proliferation (non-patent document 4). Forskolin activates adenylate cyclase and increases intracellular cAMP level. While involvement of intracellular cAMP levels in meiotic arrest is suggested, it remains unclear whether addition of forskolin alone will result in sufficient elevation of cAMP levels and proliferation of PGC.

As for induction of oogenesis, all conventional methods require somatic cells of the gonad. However, it is unclear which factors supplied by the cells actually contribute to oogenesis. In addition, since somatic cells derived from gonad in the embryonic period are used, there is a problem that collection of the somatic cells is extremely difficult in other animal species such as human.

### DOCUMENT LIST

### Patent Documents

patent document 1: WO 2012/020687

### Non-Patent Document

non-patent document 1: Hayashi, K., Ohta, H., Kurimoto, K., Aramaki, S. & Saitou, M. Reconstitution of the mouse germ cell specification pathway in culture by pluripotent stem cells. Cell 1 46, 519-532 (2011).
non-patent document 2: Hayashi, K. et al. Offspring from oocytes derived from in vitro primordial germ cell-like cells in mice. Science 338, 971-975 (2012).
non-patent document 3: Hikabe O, Hamazaki N, Nagamatsu G, Obata Y, Hirao Y, Hamada N, Shimamoto S, Imamura T, Nakashima K, Saitou M, Hayashi K. Reconstitution in vitro of the entire cycle of the mouse female germ line. Nature. 2016 Nov. 10; 539(7628):299-303.
non-patent document 4: Farini D, Scaldaferri M L, Iona S, La Sala G, De Felici M. Growth factors sustain primordial germ cell survival, proliferation and entering into meiosis in the absence of somatic cells. Dev Biol. 2005 Sep. 1; 285 (1):49-56.

### SUMMARY OF INVENTION

### Technical Problem

Therefore, the present invention aims to provide a culture system for proliferating PGC/PGCLC in vitro without using somatic cells in gonad, and a culture system capable of inducing oogenesis from the cell.

### Solution to Problem

To achieve the above-mentioned purpose, the present inventors screened about 2000 compound libraries by using PGCLC induced from mouse ESC, and found that the top 25 compounds that significantly increased proliferation of PGCLC include forskolin and retinoic acid (RA) signaling agonists known to support the proliferation of PGC, as well as a number of phosphodiesterase 4 (PDE4) inhibitors. Since the PDE4 inhibitors increase the intracellular cAMP level by inhibiting the hydrolysis of cAMP, the present inventors examined the combined effect of the inhibitor and forskolin. As a result, the PDE4 inhibitor and forskolin synergistically increased the proliferation of PGCLC to about 25-fold (about 50-fold at maximum) on average and the proliferation of E9.5 PGC to about 8-fold on average.

Furthermore, the present inventors investigated whether the proliferation efficiency of PGC/PGCLC can be further improved by further combining other compounds identified by the aforementioned screening. As a result, they have succeeded in increasing the proliferation of PGCLC and PGC (E9.5) to about 50-fold and about 16-fold on average, respectively, by using cyclosporin A in addition to PDE4 inhibitor and forskolin.

Moreover, in all cases, PGCLC progressively eliminated the DNA methylome in all genomic regions, including the parental imprint, during expansion, and faithfully reproduced the genome-wide DNA demethylation in the reproductive cells while maintaining the properties of PGC with neither sex.

Furthermore, using the above-mentioned amplified PGCLC, the present inventors studied a culture system that enables female differentiation in vitro in the absence of gonadal somatic cells. As a result, they have succeeded in inducing an oocyte-like cell by simultaneously reacting RA and bone morphogenetic protein (BMP). The oocyte-like cells showed the same meiosis image as oocytes, and comprehensive gene expression analysis revealed that the gene expression thereof closely resembles that of oocytes in the prenatal period. It was also shown that 90% or more of PGCLC can be differentiated into oocyte-like cells by this method. Importantly, the present inventors clarified that the induction of differentiation into female germ cells requires a proper competence of cell that is observed in appropriately proliferated PGC/PGCLC, but not in PGC/PGCLC immediately after induction, and characterized by demethylation of the related gene.

Based on these findings, the present inventors have constructed a model of the mechanism of sex differentiation into female in germ cells as shown in FIG. 15, and completed the present invention.

That is, the present invention provides the following.

[1] A method for expanding PGC or isolated PSC-derived PGCLC, comprising culturing PGC or PGCLC in the presence of a PDE4 inhibitor and/or cyclosporine A.
[2] The method of [1], wherein the PGC or PGCLC is cultured under a condition further comprising forskolin.
[3] A reagent for expanding PGC or PGCLC, comprising a PDE4 inhibitor and/or cyclosporine A.
[4] The reagent of [3] comprising forskolin in combination.
[5] A method for inducing oocyte from PGC or PGCLC, comprising culturing PGC or PGCLC in the presence of BMP and RA.
[6] The method of [5], wherein the BMP is one or more selected from BMP2, BMP5 and BMP7.
[7] A reagent for inducing oocyte from PGC or PGCLC, comprising BMP and RA in combination.
[8] The reagent of [7], wherein the BMP is one or more selected from BMP2, BMP5 and BMP7.
[9] A method for inducing oocyte from PGC or PGCLC, comprising
(a) expanding PGC or PGCLC by culturing the PGC or PGCLC in the presence of a PDE4 inhibitor and/or cyclosporine A, and
(b) culturing the PGC or PGCLC obtained in step (a) in the presence of BMP and RA culturing in the presence of BMP and RA.

### Advantageous Effects of Invention

According to the present invention, there is a possibility that ovum can be produced in vitro from PGC/PGCLC, and the development of basic research relating to infertility and application to assisted reproductive medicine are expected.

### BRIEF DESCRIPTION OF DRAWINGS

FIG. 1 shows the identification of compounds that stimulate PGCLC proliferation. A) Experimental procedure for compound library screening using PGCLC. B) Scatter plot of the results of compound library screening (10 μM). The fold difference (d7/d1) of the Blimp1-mVenus (BV) signal for each compound detected by Cell analyzer was plotted. Mean (red line) and 3SD (standard deviation; red dotted line) for negative control are shown. The results of PDE4 inhibitor, RAR agonist and forskolin are respectively shown in orange, blue and green. C) Stimulation of PGCLC proliferation by representative PDE4 inhibitor (GSK256066, 10 μM). Temperature distribution map (heatmap image) of the 96-well plate on day 7 of screening (upper figure) and BV fluorescence image of the well (blue square) containing GSK256066 was amplified (lower left diagram and lower right diagram). scale bar: (left Figure) 1 mm; (right Figure) 100 μm. D) Pie graph classifying the categories of the top 25 compounds (>+3SDs) in the screening (10 μM). E) Pie graph classifying the categories of 426 compounds (<−3SD) that adversely affect PGCLC proliferation/survival at screening (10 μM).

FIG. 2 shows establishment of an expansion culture system for PGCLC. A) Influence of forskolin and rolipram on PGCLC proliferation. d4 PGCLC was cultured on m220-5 feeder (NC; negative control) using a basal medium (GMEM containing 10% KSR, 2.5% FCS and 100 ng/ml SCF), and influence of 10 μM forskolin (F10), rolipram (R10) and both forskolin, rolipram (FR10) on PGCLC proliferation was examined. The number of PGCLC was counted on day 3 (c3), day 5 (c5), day 7 (c7) and day 9 (c9) of the culture. The mean fold increase in the PGCLC number at each time point relative to the number of PGCLC seeded on a plate (plated) is shown with standard deviation (n=3). B) Representative culture of d4 PGCLC containing FR10. Photographs (images of bright field (BF), Blimp1-mVenus (BV) and Stella-ECFP (SC)) and BVSC FACS plot (viable single cell in culture medium) were obtained on day 1 (d4c1), day 3 (d4c3), day 5 (d4c5), and day 7 (d4c7) of the culture. Scale bar is 100 μm. C) (left Figure) FR10 expansion of PGCLC derived from ESC strains of male (BVSC R8, BDF1-2, BCF1-2) and female (H14, H18). The fold increase in PGCLC at d4c7 versus the number of PGCLC initially plated was plotted for each experiment of each ESC strain. The average is shown with red bar. (right Figure) E9.5 PGC was amplified by FR10 as measured in the left panel. D) Cultured PGCLC (BV: green) was stained with phalloidin (red). Scale bar is 20 μm. E) Influence of FR10 on increase in intracellular cAMP concentration of d4 PGCLC. Mean values are shown with standard deviation derived from three independent experiments. F)-H) Cell cycle status of cultured PGCLCs (F) and male embryonic germ cells (G). Representative plot of cell cycle status by FACS analysis of indicated cell type is shown. The vertical axis shows BrdU incorporation, and the horizontal axis shows DNA content (7AAD). Cells in S phase, G2/M phase and G1 phase are shown in purple, blue and red, respectively, with the percentage of each population. Mean values with standard deviation derived from three independent experiments are shown in (H).

FIG. 3 shows robust spermatozoon formation by cultured PGCLC. A) 7 months after transplantation of d4c7 PGCLC derived from W/W^{v} testis (left figure, untransplanted), BDF1-2 (middle figure) or BCF1-2 (right figure) ESC strain. B)-D) Seminiferous tubules (B, C) transplanted with d4c7 PGCLC (BDF1-2) and exhibiting spermatogenesis, and the resulting spermatozoon (D). E)-G) Hematoxylin and eosin (HE) staining of transplanted testis (E, F) and caudal epididymis (G) sections. H)-K) In vitro fertilization (IVF) using spermatozoon (H) collected from the cauda epididymidis of the recipient mouse. The resulting 2-cell embryo (I) and its offspring (J, K) are shown (J with normal placenta). L)-N) Fertility of recipient W/W^{v} mice transplanted with d4c7 PGCLCs (BDF1-2). The fertility of recipient W/W^{v} mice was confirmed by natural mating (L). (M) Size of recipient W/W^{v} mouse litter transplanted with d4c7 PGCLC. The average is shown with red bar. (N) Genotypes of BV and SC transgenes in litter derived from d4c7 PGCLC. Data information: scale bar is (A) 1 mm; (B) 2 mm; (C, E) 0.5 mm; (D) 20 μm; (F, G, I) 100 μm; (H) 25 μm.

FIG. 4 shows transcriptome analysis of cultured PGCLC. A) Immunofluorescence (IF) analysis of DDX4 (upper figure), DAZL (middle figure) and OCT4 (bottom figure) levels in d4c7 PGCLCs [Blimp1-mVenus (BV) positive] was compared with E13.5 male germ cell. In the middle row, d4c7 PGCLC was outlined with a green dotted line. The ratio of the level of d4c7 PGCLC to the mean level in E13.5 male germ cells measured by densitometry [DDX4 (n=48), DAZL (n=77), OCT4 (n=61)] is shown on the right (average is shown with red bar). Scale bar is 5 μm. B) PGA of the transcriptome of the cells displayed. C), D) Venn diagrams showing overlap of up (C)/down (D) regulated genes in d4c7 PGCLC and E13.5 male/female germ cells were compared to d6 PGCLC. The number of genes in each category is shown. E) Box plot of expression level difference between d6 and d4c7 PGCLCs in the course of PGCLC culture or germ cell development (E9.5-E13.5) [median value (horizontal line), 25^{th} and 75^{th} percentiles (box) and 5^{th} and 95^{th} percentiles (error bars)] were compared to DEG d6 PGCLCs (log₂-fold difference). What is shown by the color is as indicated. F) Scatter plot of log₂ expression level changes in E13.5 germ cells (larger values in males or females) compared to d6 (x-axis) and d4c7 (y-axis) PGCLC. Genes up-regulated in d4c7 compared to d6 PGCLC are shown as red open circle (“d4c7/d6>2”) and when x>1 (i.e., up-regulated in E13.5 germ cells compared to d6 PGCLC), it is classified by the fold difference between E13.5 germ cells and d4c7 PGCLC; “fully activated at E13.5” (up-regulated in E13.5 germ cells, (yellow); “fully activated with d4c7” (within 2-fold difference, cyan color); and “over-activated with d4c7” (down-regulated in E13.5 germ cells, gray). Representative genes and selected GO terms are shown. Previously reported “germline gene” (Weber et al, 2007; Borgel et al, 2010; Kurimoto et al, 2015) or other related genes were red or blue, respectively.

FIG. 5 shows key epigenetic properties of cultured PGCLC. A) IF analysis of 5mC (upper figure), H3K27me3 (middle panel) and H3K9me2 (bottom figure) in d4c7PGCLC [Blimp1-mVenus (BV) positive] was compared with EpiLC. In the middle row, d4c7 PGCLC was outlined with a green dotted line. Relative levels of d4c7 PGCLC measured by densitometry [5mC (n=49), H3K27me3 (n=44), H3K9me2 (n=46)] and compared to the average in EpiLC are shown on the right (average is shown with red bar). Scale bar is 5 μm. B) IF analysis of DNMT1, DNMT3A, DNMT3B and UHRF1 levels in d4c7 PGCLCs (BV positive) were compared with EpiLC. In the middle row, d4c7 PGCLC was outlined with a green dotted line. Relative level in d4c7 PGCLC when compared to the average in EpiLC measured by densitometry [DNMT1 (n=57), DNMT3A (n=56), DNMT3B (n=51), UHRF1 (n=55)] is shown on the right (average is shown with red bar). Scale bar is 5 μm. C) ChIP-seq (H3K4me3, H3K27ac and H3K27me3) and 5mC level track Figure (tracks) in the 100-kb region around Prdm14 (left Figure) and Hoxb cluster (right Figure) in the indicated cell type. d4c7 PGCLCs are shaded in pink. Transcription start sites (TSSs) are indicated by dotted lines.

FIG. 6 shows elimination of DNA methylation in cultured PGCLC. A) Scatter plot comparing 5mC levels in d6, d4c3, d4c7 PGCLCs and E10.5 and E13.5 male germ cells with those in EpiLC. The 5mC levels of 2-kb unique genomic region (contour plot, upper figure), ICRs and “germline gene” (n=102) (middle figure) and repetitive consensus sequences (bottom figure) are shown. The latter two are shown along with the 5mC level of the promoter. Contour lines are drawn at intervals of 100 regions, and yellow dotted lines connect the point of origin and vertices and indicate the slope. What is shown by the color is as indicated. B) Scatter plot comparing 5mC levels between E10.5 male PGC and d4c3 PGCLC, and between E13.5 male germ cell and d4c7 PGCLC. What is shown by the color is the same as in A). C) Definition of promoters demethylated between d6 and d4c7 PGCLC; 5mC>20% for d6 and <20% for d4c7 (red open circle, n=7,737). D) Venn diagram showing the overlap between promoter DNA demethylation and genes differently expressed between d6 and d4c7 PGCLC. Between d6 and d4c7 PGCLC, demethylated promoters are classified in LCP and non-LCP. The number of genes in each category is shown.

FIG. 7 shows histone modification kinetics in cultured PGCLC. A) Scatter plot comparing log₂ H3K27ac levels between EpiLC and d6 PGCLC (left Figure), and between d6 and d4c7 PGCLC (right Figure). The H3K27ac peaks biased to d4c7 and d6 are shown in orange and cyan, respectively. B) Temperature distribution plot of correlation coefficient of H3K27ac level in pairwise comparison. What is shown by the color is as indicated. C) Definition of promoter demethylation/unmethylation in both d4c7 PGCLCs (5mC>20% in d6, <20% in d4c7, red open circle) alone, and d6 and d4c7 PGCLC (5mC<5% in d6 and d4c7, blue open circle). D) Scatter plot comparing log 2 H3K27me3 levels between d6 and d4c7 PGCLC around TSS of all genes (left figure, black), promoter demethylated only in d4c7 PGCLC (middle figure, red), and promoter demethylated/unmethylated gene in both d6 and d4c7 PGCLC (right figure, blue). E) Number of bivalent genes in ESC, EpiLC and d6 and d4c7 PGCLC. F) Changes in the displayed GO term enrichment during PGCLC induction and expansion.

FIG. 8 shows X chromosome reactivation in cultured female PGCLC. A) One X chromosome was lost in female PGCLC during PGCLC induction, (left Figure) Representative image of female EpiLC stained with Huwe1 by DNA FISH. XX and XO EpiLC were outlined with blue and orange dotted lines, respectively. (right Figure) X chromosome number derived from two female ESC lines (H14 and H18) during PGCLC induction/expansion. Scale bar is, 25 μm. B) Evaluation of X chromosome reactivation in cultured female PGCLC double stained for Huwe1 and H3K27me3. (Left Figure) Representative images of Huwe1 DNA FISH and H3K27me3 immunofluorescence. Reactivation of X chromosome was evaluated in cells maintaining two X chromosomes. (Right Figure) Analysis of Huwe1 and H3K27me3 signals in female MEF and d4/d4c3/d4c7 PGCLC. The scale bar is 5 μm. C) A model of epigenetic regulation during PGCLC induction/expansion. (Left Figure) In vivo, E9.5-E13.5, both male and female germ cells proliferate drastically (˜>100-fold expansion) (Tam & Snow, 1981; Kagiwada et al, 2013), and comprehensively erase DNA methylome. In the meantime, from around E11.5, signals from gonadal somatic cell are received, a complete “germline” gene is acquired, and male or female differentiation is started [reviewed in Spiller & Bowles, 2015]. (Right Figure) During expansion culture from d4 to d4c7 PGCLCs (˜20-fold expansion), PGCLC comprehensively eliminates DNA methylome as PGCs/germ cells in vivo. However, due to the lack of cues corresponding to the signals from gonadal somatic cell, PGCLC essentially maintains initial transcription property, at least partially, in the periphery of key genes through compensatory upregulation of H3K27me3 levels, and thus moderately acquires “germ cell line gene” and male/female property. See a summary of gene expression analyzed in this study, data set EV7 for 5mC, H3K4me3 and H3K27me3 levels.

FIG. 9 shows screening for factors that induce fate decision into female germ cell. A (left) A screening scheme for factors that induce the fate of female germ cells. Blimp1-mVenus (BV); Stella-ECFP (SC); Daz1-tdTomato (DT) (XY) or BVSC; d4/c0 PGCLC (BV (+) cells induced from mVH-RFP (VR) (XX) ESCs were sorted by FACS on m220 feeder cells and cultured in GMEM containing 10% KSR (GK10) and 2.5% fetal calf serum (FCS) in the presence of forskolin, rolipram and SCF (Ohta et al, 2017). Cytokines/chemicals for screening were provided from c3 (forskolin, rolipram and SCF were provided through culture). (Right) Expression of Daz1 and Ddx4 in d4 PGCLC and germ cells from E9.5 to E13.5 (female germ cells of E12.5 and E13.5) measured by RNA-Seq (Sasaki et al, 2015; Yamashiro et al, 2016; Ohta et al, 2017). Average of two replicates is shown. B (Upper left) FACS scheme. DT levels between BV (+) cells were analyzed at c7. (Right) FACS results for culture under the indicated conditions. Numbers indicate the percentage of DT (+) cells within the indicated gate. The concentration of cytokine shown is 500 ng/ml. (Lower left) Outline of screening results. The percentage of DT (+) cells among BV (+) cells under the indicated conditions is shown. C, D (Left) Representative FACS plot of c9 cells (BVSCVR) under the indicated conditions. The enclosed region [SC (+) cells] in the upper plot was separated from BV and VR in the lower plot. The BVSC plot of E15.5 primary oocytes is shown in C. (Right) Percentage of VR (+) cells under the indicated conditions. Means and standard deviation (SD) of two independent tests are shown. E Expression of DDX4 and SCP3 in E15.5 fetal oocytes stained with DAPI (right) and c9 RAB2 cells [BV/SC (+)] derived from BVSC ESCs (XX) (left). F Expression of TEX14 in c9 RAB2 cells [left panel: BV/SC (+)/SCP3 (+) oocyte cyst-like structure] derived from BVSC ESCs (XX) (arrow) (inset shows amplified enclosed region) and expression of TEX14 in fetal oocytes at E15.5. Scale bar is 10 μm.

FIG. 10 shows induction of female fate decision in PGCLC by BMP and RA. A Representative FACS plots of PGCLC culture at c5, c7 and c9 of control (left), and culture under conditions using RA (100 nM) (center), and using RA (100 nM) and BMP2 (300 ng/ml) (right) [top: Blimp1-mVenus (BV); Stella-ECFP (SC) expression; bottom: BV; mVH-RFP (VR) expression]. SC (+) cells enclosed in the top panel was analyzed in the bottom panel and shown with the percentage of cells in the enclosed region. B BVSC fluorescence of BV/SC (+) cells (left) and expression of SCP3/STRA8/DAZL (right) at c5, c7 and c9 in PGCLC culture using RA (100 nM) and BMP2 (300 ng/ml). Insert shows an amplified enclosed region in the left panel. Scale bar is 40 μm (left), 10 μm (left, insert Figure) and 20 μm (right). C Percentage of STRA8 (+) and SCP3 (+) cells among BV/SC (+) cells at c5, c7 and c9 based on IF analysis. Mean and standard deviation (SD) of two independent tests are shown. D Simultaneity of entering the early stage of meiosis. The vertical axis shows the number of colonies. The horizontal axis shows the percentage of SCP3 (+) cells in the BV/SC (+) colony. Colonies consisting of 2 or more cells were counted. An image of a typical colony with or without SCP3 expression is shown on the right. Scale bar is 20 μm. E Number of BV (+) cells between control culture and culture with RA (100 nM) and BMP2 (300 ng/ml). 1,500 BV (+) d4 PGCLC was seeded on day 0 of culture. One dot shows the average of 5 replicate culture wells and the bar shows the average of the dots. F Cell cycle of PGCLC at c5, c7 and c9 of culture of control, and culture under conditions using RA (100 nM), and using RA (100 nM) and BMP2 (300 ng/ml) and analyzed by EdU and 7AAD uptake. G Meiotic progression tested by spread analysis of SCP3/cH2AX/SCP1 expression at c9 using RA and BMP2 (Meuwissen et al, 1992; Yuan et al, 2000; Mahadevaiah et al, 2001). The percentage of cells at the indicated stage is shown. Scale bar is 20 μm.

FIG. 11 shows induction of female fate decision in PGCs by BMP and RA. A Scheme of PGC culture. Stella-EGFP (SG) (+) cells were cultured on m220 feeder cells using forskolin, rolipram and SCF. RA (100 nM) and/or BMP2 (300 ng/ml) were provided at c0. B Expression of DDX4/SCP3/SG at c4 under the indicated conditions. Scale bar is 20 μm. C Percentage of SCP3 (+) cells among DDX4 (+) cells. Mean and SD of replicated wells are shown. D Scheme of E11.5 fetal ovary culture. BMS493 (10 μM) and LDN1931189 (500 nM) were provided at c0. E Expression of DDX4/SCP3 at c4 under the indicated conditions. Scale bar is 20 μm. F Percentage of SCP3 (+) cells among DDX4 (+) cells. Mean and SD of two independent tests are shown. G Scheme of LDN1931189 administration (2.5 mg/kg, every 12 hr) in pregnant mice. H Expression of DDX4/SCP3 at E14.5 in embryonic ovary administered with water (control) or LDN1931189 (G). Scale bar is 20 μm. I Percentage of SCP3 (+) cells among DDX4 (+) cells. Mean and SD of two anterior and two posterior regions of the gonad are shown. J Relative mVH-RFP (VR) intensity analyzed by FACS of female or male VR (+) cells at E14.5 and treated with water (control) or LDN1931189 (G). VR intensity of control cells is 100. K Relative expression level analyzed by qPCR of female or male VR (+) cells at E14.5 and treated with water (control) or LDN1931189 (G). The level of control cells is 100. ND, not detected.

FIG. 12 shows the transcriptome during the female sex determination of PGCLC/PGC. A Heat map of expression levels of UHC (Ward method) and major genes in the indicated cells using genes with log₂ (REM+1)>2 in at least one sample (15,849 genes). ct: PGCLC cultured without RA or BMP2. Color division is shown. B PGA of germ cells in vivo (E9.5-E11.5 PGCs, E12.5-E15.5 male and female germ cells) and in vitro (cultured PGCLC). Purple dotted circles are clusters of PGC (E9.5, E10.5, E11.5) and PGCLC (c0, c3, c9). Red dotted circles are clusters of fetal oocytes (E14.5, E15.5 female germ cells) and c9 RAB2 cells. Blue, red, pink and yellow show male germ cells, female germ cells, PGCLC cultured with RA and PGCLC cultured with RAB2, respectively. C (Top) Scatter plot comparing gene expression between E14.5 male and female germ cells (left) and E14.5 female germ cells and E9.5 germ cells (right). Orange, green, red, blue and gray dots respectively indicate early PGC gene [318 gene: log₂ fold-change: E9.5-male/female E14.5>2, E9.5 log₂ (RPM+1)>4], late germ cell gene [254 gene: log₂ fold-change: male/female E14.5-E9.5>2, male/female E14.5 log₂ (RPM+1)>4], fetal oocyte gene [476 gene: log₂ fold-change: female E14.5-male E14.5>2, female E14.5-E9.5>2, female E14.5 log₂ (RPM+1) at >4], PSG gene [323 gene: log₂ fold-change: male E14.5-female E14.5>2, male E14.5-E9.5>2, male E14.5 log₂ (RPM+1)>4], and unclassified gene. (Lower left) Heat map of early PGC gene (orange), late germ cell gene (green), fetal oocyte gene (red) and PSG gene (blue) expression in germ cells in vivo and in vitro. (Lower right) GO enrichment (P value is shown) and major genes in each gene class). D Box plot of fetal oocyte gene (left) and late germ cell gene (right) levels in indicated cells [mean (horizontal line), 25 and 75 percentile (box), and 5 and 95 percentile (error bar) are shown]. E Expression of major genes during female sex determination of PGCLC/PGC [log₂ (RPM+1)]. The average of two replicates is shown. Circles filled with purple, green and orange and red circles show E9.5 PGCs, E14.5 fetal oocytes, c9 RAB2 cells and c9 RA cells, respectively. The line above the gene name is colored as shown in (C).

FIG. 13 shows the function of STRA8 in female sex determination. A representative FACS plot of wild type (WT) and Stra8 knockout (SK1) PGCLC at c3, c5, c7 and c9 using RAB2 (BVSC). B The number of Stella-ECFP(SC)(+) cells assumed by FACS in the culture shown in (A). Initial BV (+) cell count (c0) was 5,000. Mean and SD of two independent tests are shown. C Cell cycle of c9 WT and PGCLC cultured under control (upper) or RAB2 (lower) conditions, analyzed by EdU and 7AAD uptake. D PCA of the indicated cells. The arrow highlights the difference between WT and SK1 cells. Red dotted circles are clusters of fetal oocytes (E14.5, E15.5 female germ cells) and c9 RAB2 cells. E Number of DEG between WT and SK1 cells at c5, c7 and c9 cultured with RAB2 [log₂ (RPM+1)>4, log₂ (fold-change)>2]. F (Top) Comparison of scatter plots of gene expression between WT and SK1 cells, and selected GO terms of DEG. Coloring of 4 gene classes is shown. (Bottom) Non-DEG [log₂ (fold-change)<1] between fetal oocyte gene or late germ cell gene between WT and SK1 c9 RAB2 cells, and selected GO terms thereof. For fetal oocyte genes and late germ cell genes, a total of 153 (about 32.1%) and 254 (about 64.6%) of 476 genes are non-DEG, respectively. G Box plot of fetal oocyte gene, late germ cell gene and RA gene levels in indicated cells [mean (horizontal line), 25 and 75 percentile (box), and 5 and 95 percentile (error bar)]. c9 SK1 cells using c9 KO, RAB2; c9 WT cells using c9 WT cells, RAB2. H Expression of main genes in WT and SK1 cells during RAB2 culture [log₂ (RPM+1)]. Average of two replicates is shown. Coloring is shown.

FIG. 14 shows the competence of cells for fate determination of female germ cells. A experiment scheme. Ct: control; RB: d4/c0 or c7 to 48 hr, RAB2 was cultured. B PCA of the indicated cells. Yellow circles surrounded by black or red show d4/c0, c0 Ct and c0 RB or c7, c7 Ct and c7 RB cells, respectively. Black or yellow arrows indicate Ct or RB cultured cells. C Number of DEG between c0 RB and Ct cultures (left) and between c7 RB and Ct cultures (right). D GO terms for 218 genes up-regulated in c7 RB cells compared to c7 CT cells. E Scatter plot of the relationship between expression in c0 Ct/RB (left) and c7 Ct/RB (right) cells, and promoter-5mC levels in c0 (left) and c7 (right) cells (Shirane et al, 2016; Ohta et al, 2017). Red and blue circles respectively show the “meiosis” genes (GO: 0007126) (D) where the difference in % 5mC of d4/c0 and c7 PGCLC is >20% (42 genes) or <20% (110 genes). Bold letters indicate genes with log₂ (RPM+1)>5 in c0 RB cells. F Box plot of “meiosis” gene level in indicated cells [mean (horizontal line), 25 and 75 percentile (box), and 5 and 95 percentile (error bar)]. * statistically significant difference [student's t-test, P<0.05]. Coloring is the same as in (E). G Scatter plot of relationship between difference in expression of meiotic (meiosis) genes during PGC transition from early stage to late stage (E9.5 to E11.5) (left) and the difference thereof in expression during d4/c0 to c7 PGCLC culture, and late stage (right) of PGC to E13.5 female germ cell transition during c7 to c7 RAB2 PGCLC culture. Coloring and bold letters are the same as in (E). Correlation coefficient is shown. H PCA of E9.5 PGC, E11.5 PGC, E13.5 female and male germ cells, d4/c0 PGCLC, c7 PGCLC and c7 RAB2 PGCLC based on “meiosis” gene expression.

FIG. 15 shows a model of female sex determination mechanism in mouse germ cells. A Model of cell fate transition and signaling requirements for the differentiation of fetal oocyte from blastoderm. PGC identification from epiblast depends on BMP signaling (Lawson et al, 1999; Saitou et al, 2002; Ohinata et al, 2009). PGC maturation from early to late stages depends on DNA demethylation of the major promoter by passive and active mechanisms (Yamaguchi et al, 2012; Kagiwada et al, 2013) coupled with PGC propagation via SCF and cAMP signaling. Differentiation of late PGC into fetal primary oocyte depends on BMP and RA signaling. B Model of the role of BMP and RA signaling. BMP and RA signaling contributes to suppression of early PGC gene (e.g., Prdm1, Prdm14, Tfap2c, Pou5f1, Sox2, Nanog and Esrrb) and upregulation of late germ cell line gene (e.g., Ddx4, Daz1, Piwi12, Mov10 11, and Mae1) and fetal oocyte gene (e.g., Stra8, Rec8, Sycp3, Hormad1 as meiotic genes and Fig1a, Ybx2, Soh1h2 as oocyte development genes). Through BMP signaling, STRA8 promotes expression of meiotic gene and inhibits ectopic expression of developmental genes induced by RA signal transduction (RA gene). STRA8 does not significantly influence late germ cell gene or oocyte developmental gene. Without BMP signal transduction, STRA8 cannot fully upregulate meiotic genes or induce meiotic entry. BMP and RA signaling do not upregulate prespermatogonium genes.

FIG. 16 shows that cyclosporin A (CsA) can promote proliferation of PGCLC. A) Scatter plot of the results of compound library screening (left: 10 μM, right: 1 μM). The fold difference (d7/d1) of the BV signal for each compound was plotted. Mean (red line) and 3SD (standard deviation; red dotted line) for negative control are shown. The scatter plot on the left is the same scatter plot as in FIG. 1B, and shows CsA. The scatter plot on the right shows CsA after compound library screening using a concentration of 1 μM. B) Examination of CsA concentration in culture system of PGCLC. From the left, 10 μM, 5 μM, 1 μM, 0 μM CsA was applied to PGCLC, and a fold difference (d7/d1) was shown. NC: Negative target, PC: Positive target (LIF). It is clear that 5 μM CsA is optimal for the proliferation of PGCLC. C) Experimental procedure for investigating the effect of CsA. The influence of the presence or absence of CsA addition in the presence of forskolin and rolipram (FR10) was investigated. D) The influence of CsA on the proliferation of PGCLC. d4 PGCLC was cultured on m220-5 feeder using a basal medium (GMEM containing 10% KSR, 2.5% FCS and 100 ng/ml SCF), CsA (5 μM) was added to FR10 or FR10, and influence on PGCLC proliferation was examined. The number of PGCLC was counted on day 3 (c3), day 5 (c5), day 7 (c7) and day 9 (c9) of the culture. The mean fold increase in the PGCLC number at each time point relative to the number of PGCLC seeded on a plate (plated) is shown with standard deviation (n=3). E) CsA was added to FR10, representative culture of d4 PGCLC. Photographs (images of bright field (BF), Blimp1-mVenus (BV) and Stella-ECFP (SC)) were obtained on day 3 (d4c3), day 5 (d4c5), day 7 (d4c7) and day 9 (d4c3) of the culture. F) State of cell cycle of FR10 (left Figure) or PGCLCs cultured by adding CsA to FR10 (center Figure). The right figure shows the average of G1, S, and G2/M periods along with the standard deviation (n=3). G) State of cell death of PGCLCs cultured by adding FR10 (left Figure) or FR10 and CsA (center Figure). The right figure shows the average of apoptotic cells with standard deviation (n=3). H) The influence of FK506 on the proliferation of PGCLC. From the left, CsA or FK506 was applied to PGCLC at concentrations of 10 μM, 5 μM, 1 μM, and 0 μM, and fold difference (d7/d1) is shown. NC: negative target, PC: positive target (LIF).

FIG. 17 shows the effect of CsA on the gene expression, epigenetic properties and in vivo PGC of PGCLC cultured in the presence of CsA. A) PCA of transcriptome of the displayed cell. B) IF analysis of 5mC (upper figure), H3K27me3 (middle figure) and H3K9me2 (bottom figure) in d4c7PGCLC [Blimp1-mVenus (BV) positive] was compared with EpiLC. In the middle row, d4c7 PGCLC was outlined with a green dotted line. C) Relative levels of fluorescence amount of d4c7 PGCLC measure by densitometry [FR10: 5mC (n=53), H3K27me3 (n=56), H3K9me2 (n=51); +CsA: 5mC (n=53), H3K27me3 (n=59), H3K9me2 (n=57)] and compared to the average in EpiLC are shown (average is shown with red bar). D) Proliferation effect of CsA on in vivo PGC. E9.5 PGC was cultured in a culture medium added with CsA and FR10 or FR10 and the fold increase of PGCs at d4c5 was plotted against the number of PGCs plated initially (n=3).

FIG. 18 shows robust spermatozoon formation by PGCLC cultured in the presence of CsA. A)-D) Hematoxylin and eosin (HE) staining of sections of seminiferous tubules (A, B) showing spermatogenesis by transplanting d4c7 PGCLC cultured in the presence of CsA into the testis and transplanted testicles (C, D). E)-H) Microinsemination experiment (intracytoplasmic sperm injection; ICSI) using spermatozoon (E) collected from the testes of recipient mice. The resulting 2-cell embryo (F) and its offspring (G, H) are shown (G with normal placenta). I) Genotypes of BV and SC transgenes in litter derived from d4c7 PGCLC. PC: positive target. J) Body weight change of litters derived from d4c7 PGCLC (n=14). Weight changes from 1 to 4 weeks after birth are shown (average is shown with red bar).

### DESCRIPTION OF EMBODIMENTS

### [I] Expansion Method of PGC/PGCLC

The present invention provides an expansion method of PGC or isolated PSC-derived PGCLC in vitro (sometimes to be abbreviated as “method (I) of the present invention”). The method is characterized by culturing PGC or PGCLC in the presence of a PDE4 inhibitor and/or cyclosporine A.

### 1. Production of PGC/PGCLC

### 1-1. Production of PGC

PGC to be used in the present invention in the case of a mouse can be isolated, for example, from embryos of embryonic day (E) 9.5 to 11.5 by FACS or the like using the expression of a PGC-specific marker (e.g., Blimp1, Stella, etc.) as an index. The method is not limited thereto and can also be isolated by any method known per se in the art. Alternatively, PGC similarly isolated from earlier mouse embryos can be used by culturing up to the stage corresponding to mobile PGC by the method described in the following 1-2. Mammals other than mice can also be prepared in the same manner from embryos of gestational age corresponding to the gestational age of the above-mentioned mice. In the present specification, unless otherwise specified, the stages of PGC are shown below by the gestational age of the mouse embryo. In other mammals, it should be understood as the gestational age corresponding to the gestational age of the mouse embryo. Such conversion is well known in the art.

1-2. Production of PGCLC from PSC

PGCLC for use in the present invention may be any as long as it is derived in vitro from isolated PSC and has properties equivalent to those of PGC. For example, PGCLCs described in the aforementioned patent document 1 and non-patent document 1 can be mentioned. The PGCLC can be produced from isolated PSC 1 by the method shown below and via epiblast-like cells (EpiLC).

PSC for use as the starting material of PGCLC production may be any as long as it is an isolated undifferentiated cell possessing a “self-renewal” that enables it to proliferate while retaining the undifferentiated state, and “pluripotency” that enables it to differentiate into all the three primary germ layers of the embryo. As used herein, “isolated” means being placed in a living body (in vivo) or outside a living body (in vitro), and does not necessarily mean being purified. Examples of the isolated PSC include iPS cells, ES cells, embryonic germ (EG) cells, embryonic cancer (EC) cells and the like, with preference given to iPS cells or ES cells.

The method (I) of the present invention can be applied to any mammalian species in which any PSC has been or can be established. Examples of such mammals include humans, mice, rats, monkeys, dogs, pigs, bovines, cats, goat, sheep, rabbits, guinea pigs, hamsters and the like, with preference given to humans, mice, rats, monkeys, dogs and the like, more preferably humans or mice.

### (1) Preparation of Pluripotent Stem Cells

### (i) ES Cells

Pluripotent stem cells can be acquired by methods known per se. For example, available methods of preparing ES cells include, but are not limited to, methods in which a mammalian inner cell mass in the blastocyst stage is cultured [see, for example, Manipulating the Mouse Embryo: A Laboratory Manual, Second Edition, Cold Spring Harbor Laboratory Press (1994)] and methods in which an early embryo prepared by somatic cell nuclear transfer is cultured [Wilmut et al., Nature, 385, 810 (1997); Cibelli et al., Science, 280, 1256 (1998); Iritani et al., Protein, Nucleic Acid and Enzyme, 44, 892 (1999); Baguisi et al., Nature Biotechnology, 17, 456 (1999); Wakayama et al., Nature, 394, 369 (1998); Wakayama et al., Nature Genetics, 22, 127 (1999); Wakayama et al., Proc. Natl. Acad. Sci. USA, 96, 14984 (1999); RideoutIII et al., Nature Genetics, 24, 109 (2000)]. Also, ES cells can be obtained from various public and private depositories and are commercially available. For example, human ES cell lines H1 and H9 can be obtained from WiCell Institute of University of Wisconsin and KhES-1, -2 and -3 can be obtained from Institute for Frontier Medical Sciences, Kyoto University. When ES cells are produced by somatic cell nuclear transfer, the kinds and sources of somatic cells are the same as those used for producing iPS cells mentioned below.

### (ii) iPS Cells

An iPS cell can be prepared by transferring a nuclear reprogramming substance to a somatic cell.

### (a) Sources of Somatic Cells

Any cells other than germ cells of mammalian origin (e.g., mice, humans) can be used as starting material for the production of iPS cells. Examples include keratinizing epithelial cells (e.g., keratinized epidermal cells), mucosal epithelial cells (e.g., epithelial cells of the superficial layer of tongue), exocrine gland epithelial cells (e.g., mammary gland cells), hormone-secreting cells (e.g., adrenomedullary cells), cells for metabolism or storage (e.g., liver cells), intimal epithelial cells constituting interfaces (e.g., type I alveolar cells), intimal epithelial cells of the obturator canal (e.g., vascular endothelial cells), cells having cilia with transporting capability (e.g., airway epithelial cells), cells for extracellular matrix secretion (e.g., fibroblasts), constrictive cells (e.g., smooth muscle cells), cells of the blood and the immune system (e.g., T lymphocytes), sense-related cells (e.g., bacillary cells), autonomic nervous system neurons (e.g., cholinergic neurons), sustentacular cells of sensory organs and peripheral neurons (e.g., satellite cells), nerve cells and glia cells of the central nervous system (e.g., astroglia cells), pigment cells (e.g., retinal pigment epithelial cells), progenitor cells thereof (tissue progenitor cells) and the like. There is no limitation on the degree of cell differentiation; even undifferentiated progenitor cells (including somatic stem cells) and finally differentiated mature cells can be used alike as sources of somatic cells in the present invention. Examples of undifferentiated progenitor cells include tissue stem cells (somatic stem cells) such as adipose-derived stromal (stem) cells, nerve stem cells, hematopoietic stem cells, mesenchymal stem cells, and dental pulp stem cells.

The choice of mammal individual as a source of somatic cells is not particularly limited; however, when the GSCLC cells as a final product are to be used for the treatment of diseases such as infertility in humans, it is preferable, from the viewpoint of prevention of graft rejection and/or GvHD, that somatic cells are patient's own cells or collected from another person having the same or substantially the same HLA type as that of the patient. “Substantially the same HLA type” as used herein means that the HLA type of donor matches with that of patient to the extent that the transplanted cells, which have been obtained by inducing differentiation of iPS cells derived from the donor's somatic cells, can be engrafted when they are transplanted to the patient with use of immunosuppressor and the like. For example, it includes an HLA type wherein major HLAs (the three major loci of HLA-A, HLA-B and HLA-DR or four loci further including HLA-Cw) are identical (hereinafter the same meaning shall apply) and the like. When the PGC-like cells are not to be administered (transplanted) to a human, but used as, for example, a source of cells for screening for evaluating a patient's drug susceptibility or adverse reactions, it is likewise necessary to collect the somatic cells from the patient or another person with the same genetic polymorphism correlating with the drug susceptibility or adverse reactions.

Somatic cells separated from a mammal can be pre-cultured using a medium known per se suitable for the cultivation thereof, depending on the kind of the cells. Examples of such media include, but are not limited to, a minimal essential medium (MEM) containing about 5 to 20% fetal calf serum, Dulbecco's modified Eagle medium (DMEM), RPMI1640 medium, 199 medium, F12 medium, and the like. When using, for example, a transfection reagent such as a cationic liposome in contacting the cell with nuclear reprogramming substance (s) and iPS cell establishment efficiency improver(s), it is sometimes preferable that the medium be previously replaced with a serum-free medium to prevent a reduction in the transfer efficiency.

### (b) Nuclear Reprogramming Substances

In the present invention, “a nuclear reprogramming substance” refers to any substance(s) capable of inducing an iPS cell from a somatic cell, which may be composed of any substance such as a proteinous factor or a nucleic acid that encodes the same (including forms incorporated in a vector), or a low-molecular compound. When the nuclear reprogramming substance is a proteinous factor or a nucleic acid that encodes the same, the following combinations, for example, are preferable (hereinafter, only the names for proteinous factors are shown).

(1) Oct3/4, Klf4, c-Myc
(2) Oct3/4, Klf4, c-Myc, Sox2 (Sox2 is replaceable with Sox1, Sox3, Sox15, Sox17 or Sox18; Klf4 is replaceable with Klf1, Klf2 or Klf5; c-Myc is replaceable with T58A (active mutant), N-Myc, or L-Myc)
(3) Oct3/4, Klf4, c-Myc, Sox2, Fbx15, Nanog, Eras, ECAT15-2, TclI, β-catenin (active mutant S33Y)
(4) Oct3/4, Klf4, c-Myc, Sox2, TERT, SV40 Large T antigen (hereinafter SV40LT)
(5) Oct3/4, Klf4, c-Myc, Sox2, TERT, HPV16 E6
(6) Oct3/4, Klf4, c-Myc, Sox2, TERT, HPV16 E7
(7) Oct3/4, Klf4, c-Myc, Sox2, TERT, HPV6 E6, HPV16 E7
(8) Oct3/4, Klf4, c-Myc, Sox2, TERT, Bmil
[For more information on the factors shown above, see WO 2007/069666 (for information on replacement of Sox2 with Sox18 and replacement of Klf4 with Klf1 or Klf5 in the combination (2) above, see Nature Biotechnology, 26, 101-106 (2008)); for the combination “Oct3/4, Klf4, c-Myc, Sox2”, see also Cell, 126, 663-676 (2006), Cell, 131, 861-872 (2007) and the like; for the combination “Oct3/4, Klf2 (or Klf5), c-Myc, Sox2”, see also Nat. Cell Biol., 11, 197-203 (2009); for the combination “Oct3/4, Klf4, c-Myc, Sox2, hTERT, SV40 LT”, see also Nature, 451, 141-146 (2008).]

### (9) Oct3/4, Klf4, Sox2 (see Nature Biotechnology, 26, 101-106 (2008))

### (10) Oct3/4, Sox2, Nanog, Lin28 (see Science, 318, 1917-1920 (2007))

(11) Oct3/4, Sox2, Nanog, Lin28, hTERT, SV40LT (see Stem Cells, 26, 1998-2005 (2008))
(12) Oct3/4, Klf4, c-Myc, Sox2, Nanog, Lin28 (see Cell Research (2008) 600-603)
(13) Oct3/4, Klf4, c-Myc, Sox2, SV40LT (see also Stem Cells, 26, 1998-2005 (2008))

### (14) Oct3/4, Klf4 (see Nature 454:646-650 (2008), Cell Stem Cell, 2:525-528 (2008))

(15) Oct3/4, c-Myc (see Nature 454:646-650 (2008))

### (16) Oct3/4, Sox2 (see Nature, 451, 141-146 (2008), WO2008/118820)

### (17) Oct3/4, Sox2, Nanog (see WO2008/118820)

### (18) Oct3/4, Sox2, Lin28 (see WO2008/118820)

(19) Oct3/4, Sox2, c-Myc, Esrrb (Here, Essrrb can be substituted by Esrrg, see Nat. Cell Biol., 11, 197-203 (2009))
(20) Oct3/4, Sox2, Esrrb (see Nat. Cell Biol., 11, 197-203 (2009))

### (21) Oct3/4, Klf4, L-Myc

### (22) Oct3/4, Nanog

### (23) Oct3/4

(24) Oct3/4, Klf4, c-Myc, Sox2, Nanog, Lin28, SV40LT (see Science, 324: 797-801 (2009))

In (1)-(24) above, Oct3/4 may be replaced with another member of the Oct family, for example, Oct1A, Oct6 or the like. Sox2 (or Sox1, Sox3, Sox15, Sox17, Sox18) may be replaced with another member of the Sox family, for example, Sox7 or the like. Furthermore, Lin28 may be replaced with another member of the Lin family, for example, Lin28b or the like.

Any combination that does not fall in (1) to (24) above but comprises all the constituents of any one of (1) to (24) above and further comprises an optionally chosen other substance can also be included in the scope of “nuclear reprogramming substances” in the present invention. Provided that the somatic cell to undergo nuclear reprogramming is endogenously expressing one or more of the constituents of any one of (1) to (24) above at a level sufficient to cause nuclear reprogramming, a combination of only the remaining constituents excluding the one or more constituents can also be included in the scope of “nuclear reprogramming substances” in the present invention.

Of these combinations, a combination of at least one, preferably two or more, more preferably three or more, selected from among Oct3/4, Sox2, Klf4, c-Myc, Nanog, Lin28 and SV40LT, is a preferable nuclear reprogramming substance.

Particularly, when the iPS cells obtained are to be used for therapeutic purposes, a combination of the three factors Oct3/4, Sox2 and Klf4 [combination (9) above] are preferably used. When the iPS cells obtained are not to be used for therapeutic purposes (e.g., used as an investigational tool for drug discovery screening and the like), the four factors Oct3/4, Sox2, Klf4 and c-Myc, or the five factors Oct3/4, Klf4, c-Myc, Sox2 and Lin28, or the six factors consisting of the five factors and Nanog [combination (12) above], and further, the seven factors consisting of the six factors and SV40 Large T [combination (24) above] are preferable.

Furthermore, the above-described combinations wherein c-Myc is replaced with L-Myc are also preferred as nuclear reprogramming substances.

Information on the mouse and human cDNA sequences of the aforementioned nuclear reprogramming substances is available with reference to the NCBI accession numbers mentioned in WO 2007/069666 (in the publication, Nanog is described as ECAT4. Mouse and human cDNA sequence information on Lin28, Lin28b, Esrrb, Esrrg and L-Myc can be acquired by referring to the following NCBI accession numbers, respectively); those skilled in the art are easily able to isolate these cDNAs.

| Name of gene | Mouse | Human |
|---|---|---|
| Lin28 | NM_145833 | NM_024674 |
| Lin28b | NM_001031772 | NM_001004317 |
| Esrrb | NM_011934 | NM_004452 |
| Esrrg | NM_011935 | NM_001438 |
| L-Myc | NM_008506 | NM_001033081 |

A proteinous factor for use as a nuclear reprogramming substance can be prepared by inserting the cDNA obtained into an appropriate expression vector, introducing the vector into a host cell, and recovering the recombinant proteinous factor from the cultured cell or its conditioned medium. Meanwhile, when the nuclear reprogramming substance used is a nucleic acid that encodes a proteinous factor, the cDNA obtained is inserted into a viral vector, plasmid vector, episomal vector etc. to construct an expression vector, and the vector is subjected to the step of nuclear reprogramming.

### (c) Method of Transferring a Nuclear Reprogramming Substance to a Somatic Cell

Transfer of a nuclear reprogramming substance to a somatic cell can be achieved using a method known per se for protein transfer into a cell, provided that the substance is a proteinous factor. In view of human clinical applications, it is preferable that the starting material iPS cell be also prepared without gene manipulation.

Such methods include, for example, the method using a protein transfer reagent, the method using a protein transfer domain (PTD)- or cell penetrating peptide (CPP)-fusion protein, the microinjection method and the like. Protein transfer reagents are commercially available, including those based on a cationic lipid, such as BioPOTER Protein Delivery Reagent (Gene Therapy Systems), Pro-Ject™ Protein Transfection Reagent (PIERCE) and ProVectin (IMGENEX); those based on a lipid, such as Profect-1 (Targeting Systems); those based on a membrane-permeable peptide, such as Penetrain Peptide (Q biogene) and Chariot Kit (Active Motif), GenomONE (ISHIHARA SANGYO KAISHA, LTD.) utilizing HVJ envelope (inactivated hemagglutinating virus of Japan) and the like. The transfer can be achieved per the protocols attached to these reagents, a common procedure being as described below. Nuclear reprogramming substance(s) is(are) diluted in an appropriate solvent (e.g., a buffer solution such as PBS or HEPES), a transfer reagent is added, the mixture is incubated at room temperature for about 5 to 15 minutes to form a complex, this complex is added to cells after exchanging the medium with a serum-free medium, and the cells are incubated at 37° C. for one to several hours. Thereafter, the medium is removed and replaced with a serum-containing medium.

Developed PTDs include those using transcellular domains of proteins such as drosophila-derived AntP, HIV-derived TAT (Frankel, A. et al, Cell 55, 1189-93 (1988) or Green, M. & Loewenstein, P. M. Cell 55, 1179-88 (1988)), Penetratin (Derossi, D. et al, J. Biol. Chem. 269, 10444-50 (1994)), Buforin II (Park, C. B. et al. Proc. Natl Acad. Sci. USA 97, 8245-50 (2000)), Transportan (Pooga, M. et al. FASEB J. 12, 67-77 (1998)), MAP (model amphipathic peptide) (Oehlke, J. et al. Biochim. Biophys. Acta. 1414, 127-39 (1998)), K-FGF (Lin, Y. Z. et al. J. Biol. Chem. 270, 14255-14258 (1995)), Ku70 (Sawada, M. et al. Nature Cell Biol. 5, 352-7 (2003)), Prion (Lundberg, P. et al. Biochem. Biophys. Res. Commun. 299, 85-90 (2002)), pVEC (Elmquist, A. et al. Exp. Cell Res. 269, 237-44 (2001)), Pep-1 (Morris, M. C. et al. Nature Biotechnol. 19, 1173-6 (2001)), Pep-7 (Gao, C. et al. Bioorg. Med. Chem. 10, 4057-65 (2002)), SynB1 (Rousselle, C. et al. Mol. Pharmacol. 57, 679-86 (2000)), HN-I (Hong, F. D. & Clayman, G L. Cancer Res. 60, 6551-6 (2000)), and HSV-derived VP22. CPPs derived from the PTDs include polyarginines such as 11R (Cell Stem Cell, 4, 381-384 (2009)) and 9R (Cell Stem Cell, 4, 472-476 (2009)).

A fused protein expression vector incorporating cDNA of a nuclear reprogramming substance and PTD or CPP sequence is prepared, and recombination expression is performed using the vector. The fused protein is recovered and used for transfer. Transfer can be performed in the same manner as above except that a protein transfer reagent is not added.

Microinjection, a method of placing a protein solution in a glass needle having a tip diameter of about 1 μm, and injecting the solution into a cell, ensures the transfer of the protein into the cell.

However, taking into account the efficiency of establishment of iPS cells, nuclear reprogramming substance may also be used preferably in the form of a nucleic acid that encodes a proteinous factor, rather than the factor as it is. The nucleic acid may be a DNA or an RNA, or a DNA/RNA chimera, and may be double-stranded or single-stranded. Preferably, the nucleic acid is a double-stranded DNA, particularly a cDNA.

A cDNA of a nuclear reprogramming substance is inserted into an appropriate expression vector comprising a promoter capable of functioning in a host somatic cell. Useful expression vectors include, for example, viral vectors such as retrovirus, lentivirus, adenovirus, adeno-associated virus, herpesvirus and Sendai virus, plasmids for the expression in animal cells (e.g., pAl-11, pXT1, pRc/CMV, pRc/RSV, pcDNAI/Neo) and the like.

A vector for this purpose can be chosen as appropriate according to the intended use of the iPS cell to be obtained. Useful vectors include adenovirus vector, plasmid vector, adeno-associated virus vector, retrovirus vector, lentivirus vector, Sendai virus vector, episomal vector and the like.

Examples of promoters used in expression vectors include the EF1α promoter, the CAG promoter, the SRα promoter, the SV40 promoter, the LTR promoter, the CMV (cytomegalovirus) promoter, the RSV (Rous sarcoma virus) promoter, the MoMuLV (Moloney mouse leukemia virus) LTR, the HSV-TK (herpes simplex virus thymidine kinase) promoter and the like, with preference given to the EF1α promoter, the CAG promoter, the MoMuLV LTR, the CMV promoter, the SRα promoter and the like.

The expression vector may contain as desired, in addition to a promoter, an enhancer, a polyadenylation signal, a selectable marker gene, a SV40 replication origin and the like. Examples of selectable marker genes include the dihydrofolate reductase gene, the neomycin resistant gene, the puromycin resistant gene and the like.

The nucleic acids as nuclear reprogramming substances (reprogramming genes) may be separately integrated into different expression vectors, or 2 kinds or more, preferably 2 to 3 kinds, of genes may be incorporated into a single expression vector. Preference is given to the former case with the use of a retrovirus or lentivirus vector, which offers high gene transfer efficiency, and to the latter case with the use of a plasmid, adenovirus, or episomal vector and the like. Furthermore, an expression vector incorporating two kinds or more of genes and another expression vector incorporating one gene alone can be used in combination.

In the context above, when a plurality of genes are incorporated in one expression vector, these genes can preferably be inserted into the expression vector via an intervening sequence enabling polycistronic expression. By using an intervening sequence enabling polycistronic expression, it is possible to more efficiently express a plurality of genes incorporated in one kind of expression vector. Useful sequences enabling polycistronic expression include, for example, the 2A sequence of foot-and-mouth disease virus (PLoS ONE 3, e2532, 2008, Stem Cells 25, 1707, 2007), IRES sequence (U.S. Pat. No. 4,937,190) and the like, with preference given to the 2A sequence.

An expression vector harboring a nucleic acid as a nuclear reprogramming substance can be introduced into a cell by a technique known per se according to the choice of the vector. In the case of a viral vector, for example, a plasmid containing the nucleic acid is introduced into an appropriate packaging cell (e.g., Plat-E cells) or a complementary cell line (e.g., 293-cells), the viral vector produced in the culture supernatant is recovered, and the vector is infected to the cell by a method suitable for the viral vector. For example, specific means using a retroviral vector are disclosed in WO2007/69666, Cell, 126, 663-676 (2006) and Cell, 131, 861-872 (2007). Specific means using a lentivirus vector is disclosed in Science, 318, 1917-1920 (2007). When PGC-like cells induced from iPS cells are utilized for regenerative medicine such as treatment of infertility and gene therapy of germ cells, an expression (reactivation) of a reprogramming gene potentially increases the risk of carcinogenesis in germ cells or reproductive tissues regenerated from PGC-like cells derived from iPS cells; therefore, a nucleic acid encoding a nuclear reprogramming substance is preferably expressed transiently, without being integrated into the chromosome of the cells. From this viewpoint, use of an adenoviral vector, whose integration into chromosome is rare, is preferred. Specific means using an adenoviral vector is disclosed in Science, 322, 945-949 (2008). Because an adeno-associated viral vector is also low in the frequency of integration into chromosome, and is lower than adenoviral vectors in terms of cytotoxicity and inflammation-inducibility, it can be mentioned as another preferred vector. Because Sendai viral vector is capable of being stably present outside the chromosome, and can be degraded and removed using an siRNA as required, it is preferably utilized as well. Regarding a Sendai viral vector, one described in J. Biol. Chem., 282, 27383-27391 (2007) and JP-3602058 B can be used.

When a retroviral vector or a lentiviral vector is used, even if silencing of the transgene has occurred, it possibly becomes reactivated; therefore, for example, a method can be used preferably wherein a nucleic acid encoding a nuclear reprogramming substance is cut out using the Cre-loxP system, when becoming unnecessary. That is, with loxP sequences arranged on both ends of the nucleic acid in advance, iPS cells are induced, thereafter the Cre recombinase is allowed to act on the cells using a plasmid vector or adenoviral vector, and the region sandwiched by the loxP sequences can be cut out. Because the enhancer-promoter sequence of the LTR U3 region possibly upregulates a host gene in the vicinity thereof by insertion mutation, it is more preferable to avoid the expression regulation of the endogenous gene by the LTR outside of the loxP sequence remaining in the genome without being cut out, using a 3′-self-inactivating (SIN) LTR prepared by deleting the sequence, or substituting the sequence with a polyadenylation sequence such as of SV40. Specific means using the Cre-loxP system and SIN LTR is disclosed in Chang et al., Stem Cells, 27: 1042-1049 (2009).

Meanwhile, being a non-viral vector, a plasmid vector can be transferred into a cell using the lipofection method, liposome method, electroporation method, calcium phosphate co-precipitation method, DEAE dextran method, microinjection method, gene gun method and the like. Specific means using a plasmid as a vector are described in, for example, Science, 322, 949-953 (2008) and the like.

When a plasmid vector, an adenovirus vector and the like are used, the transfection can be performed once or more optionally chosen times (e.g., once to 10 times, once to 5 times or the like). When two or more kinds of expression vectors are introduced into a somatic cell, it is preferable that these all kinds of expression vectors be concurrently introduced into a somatic cell; however, even in this case, the transfection can be performed once or more optionally chosen times (e.g., once to 10 times, once to 5 times or the like), preferably the transfection can be repeatedly performed twice or more (e.g., 3 times or 4 times).

Also when an adenovirus or a plasmid is used, the transgene can get integrated into chromosome; therefore, it is eventually necessary to confirm the absence of insertion of the gene into chromosome by Southern blotting or PCR. For this reason, like the aforementioned Cre-loxP system, it can be advantageous to use a means wherein the transgene is integrated into chromosome, thereafter the gene is removed. In another preferred mode of embodiment, a method can be used wherein the transgene is integrated into chromosome using a transposon, thereafter a transposase is allowed to act on the cell using a plasmid vector or adenoviral vector so as to completely eliminate the transgene from the chromosome. As examples of preferable transposons, piggyBac, a transposon derived from a lepidopterous insect, and the like can be mentioned. Specific means using the piggyBac transposon is disclosed in Kaji, K. et al., Nature, 458: 771-775 (2009), Woltjen et al., Nature, 458: 766-770 (2009).

Another preferred non-recombination type vector is an episomal vector autonomously replicable outside the chromosome. A specific procedure for using an episomal vector is disclosed by Yu et al. in Science, 324, 797-801 (2009). As required, an expression vector may be constructed by inserting a reprogramming gene into an episomal vector having loxP sequences placed in the same orientation at both the 5′ and 3′ sides of the vector element essential for the replication of the episomal vector, and this may be transferred into a somatic cell.

Examples of the episomal vector include vectors comprising a sequence required for its autonomous replication, derived from EBV, SV40 and the like, as a vector element. Specifically, the vector element required for its autonomous replication is a replication origin or a gene that encodes a protein that binds to the replication origin to regulate its replication; examples include the replication origin oriP and EBNA-1 gene for EBV, and the replication origin ori and SV40 large T antigen gene for SV40.

The episomal expression vector contains a promoter that controls the transcription of the reprogramming gene. The promoter used can be the same promoter as the above. The episomal expression vector may further comprise an enhancer, poly-A addition signal, selection marker gene and the like as desired, as described above. Examples of selection marker gene include the dihydrofolate reductase gene, neomycin resistance gene and the like.

An episomal vector can be introduced into a cell using, for example, lipofection method, liposome method, electroporation method, calcium phosphate co-precipitation method, DEAE dextran method, microinjection method, gene gun method and the like. Specifically, the method described in Science, 324: 797-801 (2009), for example, can be used.

Whether or not the vector element required for replication of reprogramming gene has been removed from the iPS cell can be determined by performing Southern blot analysis or PCR analysis using a part of the vector as a probe or primer, with an episome fraction isolated from the iPS cell as the template, to examine for the presence or absence of a band or the length of the band detected. An episome fraction can be prepared using a method well known in the art, for example, the method described in Science, 324: 797-801 (2009).

When the nuclear reprogramming substance is a low-molecular compound, introduction thereof into a somatic cell can be achieved by dissolving the substance at an appropriate concentration in an aqueous or non-aqueous solvent, adding the solution to a medium suitable for cultivation of somatic cells isolated from human or mouse [e.g., minimal essential medium (MEM) comprising about 5 to 20% fetal bovine serum, Dulbecco's modified Eagle medium (DMEM), RPMI1640 medium, 199 medium, F12 medium, and the like] so that the nuclear reprogramming substance concentration will fall in a range that is sufficient to cause nuclear reprogramming in somatic cells and does not cause cytotoxicity, and culturing the cells for a given period. The nuclear reprogramming substance concentration varies depending on the kind of nuclear reprogramming substance used, and is chosen as appropriate over the range of about 0.1 nM to about 100 nM. Duration of contact is not particularly limited, as far as it is sufficient to cause nuclear reprogramming of the cells; usually, the nuclear reprogramming substance may be allowed to be co-present in the medium until a positive colony emerges.

### (d) iPS Cell Establishment Efficiency Improvers

In recent years, various substances that improve the efficiency of establishment of iPS cells, which has traditionally been low, have been proposed one after another. When brought into contact with a somatic cell together with the aforementioned nuclear reprogramming substances, these establishment efficiency improvers are expected to further raise the efficiency of establishment of iPS cells.

Examples of iPS cell establishment efficiency improvers include, but are not limited to, histone deacetylase (HDAC) inhibitors [e.g., valproic acid (VPA) (Nat. Biotechnol., 26(1): 795-797 (2008)], low-molecular inhibitors such as trichostatin A, sodium butyrate, MC 1293, and M344, nucleic acid-based expression inhibitors such as siRNAs and shRNAs against HDAC (e.g., HDAC1 siRNA Smartpool® (Millipore), HuSH 29mer shRNA Constructs against HDAC1 (OriGene) and the like), and the like], DNA methyltransferase inhibitors (e.g., 5′-azacytidine) [Nat. Biotechnol., 26(1): 795-797 (2008)], G9a histone methyltransferase inhibitors [e.g., low-molecular inhibitors such as BIX-01294 (Cell Stem Cell, 2: 525-528 (2008)), nucleic acid-based expression inhibitors such as siRNAs and shRNAs against G9a (e.g., G9a siRNA (human) (Santa Cruz Biotechnology) and the like) and the like], L-channel calcium agonists (e.g., Bayk8644) [Cell Stem Cell, 3, 568-574 (2008)], p53 inhibitors [e.g., siRNA and shRNA against p53 (Cell Stem Cell, 3, 475-479 (2008)), UTF1 [Cell Stem Cell, 3, 475-479 (2008)], Wnt Signaling (e.g., soluble Wnt3a) [Cell Stem Cell, 3, 132-135 (2008)], 2i/LIF [2i is an inhibitor of mitogen-activated protein kinase signaling and glycogen synthase kinase-3, PloS Biology, 6(10), 2237-2247 (2008)] and the like. As mentioned above, the nucleic acid-based expression inhibitors may be in the form of expression vectors harboring a DNA that encodes an siRNA or shRNA.

Among the constituents of the aforementioned nuclear reprogramming substances, SV40 large T and the like, for example, can also be included in the scope of iPS cell establishment efficiency improvers because they are deemed not so essential, but auxiliary, factors for somatic cell nuclear reprogramming. In the situation of the mechanisms for nuclear reprogramming remaining unclear, the auxiliary factors, which are not essential for nuclear reprogramming, may be conveniently considered as nuclear reprogramming substances or iPS cell establishment efficiency improvers. Hence, because the somatic cell nuclear reprogramming process is understood as an overall event resulting from contact of nuclear reprogramming substance(s) and iPS cell establishment efficiency improver(s) with a somatic cell, it seems unnecessary for those skilled in the art to always distinguish between the nuclear reprogramming substance and the iPS cell establishment efficiency improver.

Contact of an iPS cell establishment efficiency improver with a somatic cell can be achieved as described above for each of three cases: (a) the improver is a proteinous factor, (b) the improver is a nucleic acid that encodes the proteinous factor, and (c) the improver is a low-molecular compound.

An iPS cell establishment efficiency improver may be brought into contact with a somatic cell simultaneously with a nuclear reprogramming substance, or either one may be contacted in advance, as far as the efficiency of establishment of iPS cells from the somatic cell is significantly improved, compared with the absence of the improver. In an embodiment, for example, when the nuclear reprogramming substance is a nucleic acid that encodes a proteinous factor and the iPS cell establishment efficiency improver is a chemical inhibitor, the iPS cell establishment efficiency improver can be added to the medium after the cell is cultured for a given length of time after the gene transfer treatment, because the nuclear reprogramming substance involves a given length of time lag from the gene transfer treatment to the mass-expression of the proteinous factor, whereas the iPS cell establishment efficiency improver is capable of rapidly acting on the cell. In another embodiment, when a nuclear reprogramming substance and an iPS cell establishment efficiency improver are both used in the form of a viral or plasmid vector, for example, both may be simultaneously introduced into the cell.

### (e) Improving the Establishment Efficiency by Culture Conditions

The efficiency of establishment of iPS cells can be further improved by culturing the somatic cells therefor under hypoxic conditions in the step of nuclear reprogramming of the cells. The term hypoxic conditions as used herein means that the oxygen concentration in the ambient atmosphere during cell culture is significantly lower than that in the air. Specifically, such conditions include lower oxygen concentrations than the oxygen concentrations in the ambient atmosphere of 5-10% CO₂/95-90% air, which is commonly used for ordinary cell culture; for example, oxygen concentrations of 18% or less in the ambient atmosphere are applicable. Preferably, the oxygen concentration in the ambient atmosphere is 15% or less (e.g., 14% or less, 13% or less, 12% or less, 11% or less and the like), 10% or less (e.g., 9% or less, 8% or less, 7% or less, 6% or less and the like), or 5% or less (e.g., 4% or less, 3% or less, 2% or less and the like). The oxygen concentration in the ambient atmosphere is preferably 0.1% or more (e.g., 0.2% or more, 0.3% or more, 0.4% or more and the like), 0.5% or more (e.g., 0.6% or more, 0.7% or more, 0.8% or more, 0.9% or more and the like), or 1% or more (e.g., 1.1% or more, 1.2% or more, 1.3% or more, 1.4% or more and the like).

There is no limitation on how to create hypoxic conditions in a cellular environment; the easiest of suitable methods is to culture cells in a CO₂ incubator that allows control of oxygen concentrations. Such CO₂ incubators are commercially available from a number of manufacturers of equipment (e.g., CO₂ incubators for hypoxic culture manufactured by Thermo Scientific, Ikemoto Scientific Technology, Juji Field Inc., and Wakenyaku Co., Ltd. can be used).

The timing of beginning cell culture under hypoxic conditions is not particularly limited, as far as it does not interfere with improving the efficiency of establishment of iPS cells compared with that obtained at a normal oxygen concentration (20%). The starting time may be before or after contact of nuclear reprogramming substances with a somatic cell, and may be at the same time as the contact. For example, it is preferable that cell culture under hypoxic conditions be begun just after contacting a nuclear reprogramming substance with a somatic cell, or after a given time (e.g., 1 to 10 (e.g., 2, 3, 4, 5, 6, 7, 8 or 9) days) following the contact.

The duration of cell culture under hypoxic conditions is not particularly limited, as far as it does not interfere with improving the efficiency of establishment of iPS cells compared with that obtained at a normal oxygen concentration (20%); examples include, but are not limited to, between 3 days or more, 5 days or more, 7 days or more or 10 days or more, and 50 days or less, 40 days or less, 35 days or less or 30 days or less. The preferred duration of cell culture under hypoxic conditions also varies depending on the oxygen concentration in the ambient atmosphere; those skilled in the art can adjust as appropriate the duration of cell culture according to the oxygen concentration used. In an embodiment of the present invention, when iPS cell candidate colonies are selected with drug resistance as an indicator, it is preferable that a normal oxygen concentration be restored from hypoxic conditions by the start of drug selection.

Furthermore, the preferred starting time and duration of cell culture under hypoxic conditions also vary depending on the choice of nuclear reprogramming substances used, the efficiency of establishment of iPS cells under conditions involving a normal oxygen concentration, and other factors.

After the nuclear reprogramming substance(s) (and iPS cell establishment efficiency improver(s)) is(are) brought into contact with the cell, the cell can be cultured under conditions suitable for the cultivation of, for example, ES cells. In the case of mouse cells, the cultivation is carried out with the addition of Leukemia Inhibitory Factor (LIF) as a differentiation suppressor to an ordinary medium. Meanwhile, in the case of human cells, it is desirable that basic fibroblast growth factor (bFGF) and/or stem cell factor (SCF) be added in place of LIF. Usually, the cells are cultured in the co-presence of mouse embryo-derived fibroblasts (MEFs) treated with radiation or an antibiotic to terminate the cell division thereof, as feeder cells. Usually, STO cells and the like are commonly used as MEFs, but for inducing iPS cells, SNL cells [McMahon, A. P. & Bradley, A. Cell 62, 1073-1085 (1990)] and the like are commonly used. Co-culture with feeder cells may be started before contact of the nuclear reprogramming substance, at the time of the contact, or after the contact (e.g., 1-10 days later).

A candidate colony of iPS cells can be selected by a method with drug resistance and reporter activity as indicators, and also by a method based on visual examination of morphology.

As an example of the former, a colony positive for drug resistance and/or reporter activity is selected using a recombinant somatic cell wherein a drug resistance gene and/or a reporter gene is targeted to the locus of a gene highly expressed specifically in pluripotent cells (e.g., Fbx15, Nanog, Oct3/4 and the like, preferably Nanog or Oct3/4). Examples of such recombinant somatic cells include MEFs from a mouse having the βgeo (which encodes a fusion protein of β-galactosidase and neomycin phosphotransferase) gene knocked-in to the Fbx15 locus [Takahashi & Yamanaka, Cell, 126, 663-676 (2006)], MEFs from a transgenic mouse having the green fluorescent protein (GFP) gene and the puromycin resistance gene integrated in the Nanog locus [Okita et al., Nature, 448, 313-317 (2007)] and the like. Meanwhile, examples of the method of selecting candidate colonies based on visual examination of morphology include the method described by Takahashi et al. in Cell, 131, 861-872 (2007). Although the method using reporter cells is convenient and efficient, it is desirable from the viewpoint of safety that colonies be selected by visual examination when iPS cells are prepared for the purpose of human treatment. When the three factors Oct3/4, Klf4 and Sox2 are used as nuclear reprogramming substances, the number of clones established decreases but the resulting colonies are mostly of iPS cells of high quality comparable to ES cells, so that iPS cells can efficiently be established even without using reporter cells.

The identity of the cells of a selected colony as iPS cells can be confirmed by positive responses to a Nanog (or Oct3/4) reporter (puromycin resistance, GFP positivity and the like) as well as by the formation of a visible ES cell-like colony, as described above. However, to ensure higher accuracy, it is possible to perform tests such as analyzing the expression of various ES-cell-specific genes and transplanting the cells selected to a mouse and confirming the formation of teratomas.

(iii) Naive Human ES and iPS Cells

Conventional human ES cells derived from blastocyst-stage embryos have very different biological (morphological, molecular and functional) properties from mouse ES cells. Mouse pluripotent stem cells can exit in two functionally distinct states, LIF-dependent ES cells and bFGF-dependent epiblast stem cells (EpiSCs). Molecular analyses suggest that the pluripotent state of human ES cells is similar to that of mouse EPiSCs rather than that of mouse ES cells. Recently, human ES and iPS cells in a mouse ES cell-like pluripotent state (also referred to as naive human ES and iPS cells) have been established by ectopic induction of Oct3/4, Sox2, Klf4, c-Myc and Nanog in the presence of LIE (see Cell Stem Cells, 6: 535-546, 2010), or ectopic induction of Oct3/4, Klf4 and Klf2 combined with LIE and inhibitors of GSK3β and ERK1/2 pathway (see Proc. Natl. Acad. Sci. USA, online publication doi/10.1073/pnas.1004584107). These naive human ES and iPS cells may be preferable starting materials for the present invention due to their pluripotent more immature compared to that of conventional human ES and iPS cells.

(2) Induction of Differentiation from PSC to EpiLCs

Basal media for differentiation induction include, but are not limited to, Neurobasal medium, Neural Progenitor Basal medium, NS-A medium, BME medium, BGJb medium, CMRL 1066 medium, minimal essential medium (MEM), Eagle MEM, αMEM, Dulbecco's modified Eagle medium (DMEM), Glasgow MEM, Improved MEM Zinc Option medium, IMDM medium, 199 medium, DMEM/F12 medium, Ham's medium, RPMI1640 medium, Fischer's medium, and mixtures thereof.

The medium can be a serum-containing or serum-free medium. Preferably, a serum-free medium can be used. The serum-free medium (SFM) refers to media with no unprocessed or unpurified serum and accordingly, can include media with purified blood-derived components or animal tissue-derived components (such as growth factors). The concentration of serum (for example, fetal bovine serum (FBS), human serum, etc.) can be 0-20%, preferably 0-5%, more preferably 0-2%, most preferably 0% (i.e., serum-free). The SEM may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′thiolglycerol, or equivalents thereto. The alternatives to serum can be prepared by the method disclosed in WO 98/30679, for example. Alternatively, any commercially available materials can be used for more convenience. The commercially available materials include Knockout™ Serum Replacement (KSR), Chemically-defined Lipid concentrated, and Glutamax (Invitrogen).

The medium can also contain other additives known per se. The additive is not subject to limitation, as long as EpiLCs equivalent to pre-gastrulating epiblast cells can be produced by the method of the present invention; for example, growth factors (for example, insulin and the like), polyamines (for example, putrescine and the like), minerals (for example, sodium selenate and the like), saccharides (for example, glucose and the like), organic acids (for example, pyruvic acid, lactic acid and the like), amino acids (for example, non-essential amino acids (NEAA), L-glutamine and the like), reducing agents (for example, 2-mercaptoethanol and the like), vitamins (for example, ascorbic acid, d-biotin and the like), steroids (for example, [beta]-estradiol, progesterone and the like), antibiotics (for example, streptomycin, penicillin, gentamycin and the like), buffering agents (for example, HEPES and the like), nutritive additives (for example, B27 supplement, N2 supplement, StemPro-Nutrient Supplement and the like) and the like can be mentioned. It is preferable that each of the additives be contained in a concentration range known per se.

In the method of producing EpiLCs of the present invention, pluripotent stem cells may be cultured in the presence or absence of feeder cells. The feeder cells are not subject to limitation, as long as EpiLCs can be produced by the method of the present invention. Feeder cells known per se for use in culturing pluripotent stem cells such as ESCs and iPSCs can be used. For example, fibroblasts (mouse embryonic fibroblasts, mouse fibroblast cell line STO and the like) can be mentioned. The feeder cells are preferably inactivated by a method known per se, for example, radiation (gamma rays and the like), treatment with an anticancer agent (mitomycin C and the like) and the like. However, in a preferred embodiment of the present invention, pluripotent stem cells are cultured under feeder-free conditions.

The medium for inducing differentiation from pluripotent stem cells to EpiLCs (medium A) contains activin A as an essential additive in the basal medium. The activin A concentration is, for example, about 5 ng/ml or more, preferably about 10 ng/ml or more, more preferably about 15 ng/ml or more, and is, for example, about 40 ng/ml or less, preferably about 30 ng/ml or less, more preferably 25 ng/ml or less.

The medium A preferably further contains bFGF and/or KSR. bFGF and KSR remarkably increase the induction efficiency for EpiLCs when present in a range of effective concentrations. The bFGF concentration is, for example, about 5 ng/ml or more, preferably about 7.5 ng/ml or more, more preferably about 10 ng/ml or more, and is, for example, about 30 ng/ml or less, preferably about 20 ng/ml or less, more preferably about 15 ng/ml or less. The KSR concentration is, for example, about 0.1 w/w % or more, preferably about 0.3 w/w % or more, more preferably about 0.5 w/w % or more, and is, for example, about 5 w/w % or less, preferably about 3 w/w % or less, more preferably about 2 w/w % or less.

In a particularly preferred embodiment, the medium A contains activin A, bFGF and KSR in addition to the basal medium. Appropriate concentrations of these ingredients can be chosen over the range of about 10-30 ng/ml, preferably 15-25 ng/ml for activin A, about 7.5-20 ng/ml, preferably about 10-15 ng/ml for bFGF, and about 0.3-3 w/w %, preferably about 0.5-2 w/w % for KSR.

The activin A and bFGF contained in the medium A are not subject to limitation as to the source thereof, may be isolated and purified from cells of any mammals (for example, human, mouse, monkey, swine, rat, dog and the like). It is preferable to use activin A and bFGF homologous to the pluripotent stem cells subjected to the culture. The activin A and bFGF may also be chemically synthesized or biochemically synthesized using a cell-free translation system, or produced from a transformant bearing a nucleic acid encoding each of the proteins. The recombinant products of activin A and bFGF are commercially available.

A culture vessel used for inducing pluripotent stem cells into EpiLCs can include, but is particularly not limited to, flask, flask for tissue culture, dish, petri dish, dish for tissue culture, multi dish, micro plate, micro-well plate, multi plate, multi-well plate, micro slide, chamber slide, schale, tube, tray, culture bag, and roller bottle. The culture vessel can be cellular adhesive. The cellular adhesive culture vessel can be coated with any of substrates for cell adhesion such as extracellular matrix (ECM) to improve the adhesiveness of the vessel surface to the cells. The substrate for cell adhesion can be any material intended to attach pluripotent stem cells or feeder cells (if used). The substrate for cell adhesion includes collagen, gelatin, poly-L-lysine, poly-D-lysine, poly-L-ornithine, laminin, and fibronectin and mixtures thereof for example Matrigel, and lysed cell membrane preparations (Klimanskaya I et al 2005. Lancet 365: p 1636-1641).

In this cultivation, pluripotent stem cells are plated onto the culture vessel mentioned above to obtain a cell density of, for example, about 10⁴-10⁵ cells/cm², preferably about 2 to 8×10⁴ cells/cm², and cultured in an incubator under atmospheric conditions of 1-10% CO₂/99-90% air at about 30-40° C., preferably about 31° C., for less than 3 days, preferably about 2 days (e.g., 48±12 hours, preferably 48±6 hours). As a result of the culture, cells with flattened epiblast-like structure uniformly emerges.

The fact of differentiation into EpiLCs can be confirmed by, for example, analyzing the expression levels of EPiLC- and/or pluripotent stem cell-marker genes using RT-PCR. The EpiLC of the present invention means a cell in E5.5-E6.0 epiblast-like (pre-gastrulating epiblast-like) state. To be specific, the EpiLC is defined as a cell having either or both of the following properties:

(1) elevated gene expression of at least one selected from Fgf5, Wnt3 and Dnmt3b compared to the pluripotent stem cell before inducing differentiation;
(2) reduced gene expression of at least one selected from Gata4, Gata6, Sox17 and Blimp1 compared to the pluripotent stem cell before inducing differentiation. Therefore, the fact of differentiation into EpiLCs can be confirmed by determining the expression levels of at least one selected from Fgf5, Wnt3 and Dnmt3b and/or at least one selected from Gata4, Gata6, Sox17 and Blimp1 in the cells obtained by the culture, and comparing the expression levels with those in the pluripotent stem cells before inducing differentiation.

More preferably, the EpiLC of the present invention has the following properties:

(1) continuous gene expression of Oct3/4;
(2) reduced gene expression of Sox2 and Nanog compared to the pluripotent stem cell before inducing differentiation;
(3) elevated gene expression of Fgf5, Wnt3 and Dnmt3b compared to the pluripotent stem cell before inducing differentiation; and
(4) reduced gene expression of Gata4, Gata6, Sox17 and Blimp1 compared to the pluripotent stem cell before inducing differentiation.

As mentioned above, in a preferred embodiment, the medium A of the present invention contains activin A, bFGF and KSR. Accordingly, the present invention also provides a reagent kit for inducing the differentiation from pluripotent stem cells to EpiLCs comprising activin A, bFGF and KSR. These ingredients may be supplied in a state dissolved in water or an appropriate buffer solution, and may also be supplied as a lyophilized powder which may be used after being freshly dissolved in an appropriate solvent. These ingredients may be supplied as individual reagents in respective kits, and, as far as they do not adversely affect each other, they can be supplied as a single mixed reagent of 2 kinds or more.

(3) Induction of Differentiation from EpiLCs to PGCLC Cells

By culturing thus-obtained EpiLCs in the presence of BMP4 and LIF, it is possible to induce differentiation into PGC-like cells (Cell, 137, 571-584 (2009)). Accordingly, a second aspect of the present invention relates to a method of producing PGC-like cells from pluripotent stem cells through EpiLCs obtained by the method of (2) above. Namely, the method comprises:

I) the step for producing an EpiLC from pluripotent stem cells according to any of the methods described in (2) above; and
II) the step for culturing the EpiLC obtained in the step I) in the presence of BMP4 and LIF.

As the basal medium for differentiation induction in the step II), the basal media exemplified for the use in the step I) are likewise preferably used. The medium may contain the same additives as those exemplified for the use in the step I), as long as PGC-like cells capable of contributing to normal spermatogenesis can be produced by the method of the present invention.

The medium can be a serum-containing or serum-free medium (SFM). Preferably, a serum-free medium can be used. The concentration of serum (for example, fetal bovine serum (FBS), human serum, etc.) can be 0-20%, preferably 0-5%, more preferably 0-2%, most preferably 0% (i.e., serum-free). The SFM may contain or may not contain any alternatives to serum such as KSR.

The medium for inducing differentiation from EpiLCs to PGC-like cells (medium B) contains bone morphogenetic protein 4 (BMP4) and leukemia inhibitory factor (LIF) as an essential additive in the basal medium. The concentration of BMP4 is, for example, about 100 ng/ml or more, preferably about 200 ng/ml or more, more preferably about 300 ng/ml or more. Also, the concentration of BMP4 is, for example, about 1,000 ng/ml or less, preferably about 800 ng/ml or less, more preferably 600 ng/ml or less. The concentration of LIF is, for example, about 300 U/ml or more, preferably about 500 U/ml or more, more preferably about 800 U/ml or more. Also, the concentration of LIF is, for example, about 2,000 U/ml or less, preferably about 1,500 U/ml or less, more preferably 1,200 U/ml or less.

The medium B preferably further contains at least one additive(s) selected from stem cell factor (SCF), bone morphogenetic protein 8b (BMP8b) and epidermal growth factor (EGF). SCF, BMP8b and EGF remarkably prolong the period of time which PGC-like cells are maintained in a Blimp1- and Stella-positive state, when present in ranges of effective concentrations. The concentration of SCF is, for example, about 30 ng/ml or more, preferably about 50 ng/ml or more, more preferably about 80 ng/ml or more. Also, the concentration of SCF is, for example, about 200 ng/ml or less, preferably about 150 ng/ml or less, more preferably about 120 ng/ml or less. The concentration of BMP8b is, for example, about 100 ng/ml or more, preferably about 200 ng/ml or more, more preferably about 300 ng/ml or more. Also, the concentration of BMP8b is, for example, about 1,000 ng/ml or less, preferably about 800 ng/ml or less, more preferably 600 ng/ml or less. The concentration of EGF is, for example, about 10 ng/ml or more, preferably about 20 ng/ml or more, more preferably about 30 ng/ml or more. Also, the concentration of EGF is, for example, about 100 ng/ml or less, preferably about 80 ng/ml or less, more preferably about 60 ng/ml.

In a particularly preferred embodiment, the medium B contains BMP, LIF, SCF, BMP8b and EGF in addition to the basal medium. The concentrations of these ingredients can be chosen as appropriate over the ranges of about 200-800 ng/ml, preferably about 300-600 ng/ml for BMP4, about 500-1500 U/ml, preferably about 800-1,200 U/ml for LIF, about 50-150 ng/ml, preferably about 80-120 ng/ml for SCF, about 200-800 ng/ml, preferably about 300-600 ng/ml for BMP8b, and about 20-80 ng/ml, preferably about 30-60 ng/ml for EGF.

The BMP4, LIF, SCF, BMP8b and EGF contained in the medium B are not subject to limitation as to the source thereof, may be isolated and purified from cells of any mammals (for example, human, mouse, monkey, swine, rat, dog and the like). It is preferable to use BMP4, LIF, SCF, BMP8b and EGF homologous to the EpiLCs subjected to the culture. The BMP4, LIF, SCF, BMP8b and EGF may also be chemically synthesized or biochemically synthesized using a cell-free translation system, or produced from a transformant bearing a nucleic acid encoding each of the proteins. The recombinant products of BMP4, LIF, SCF, BMP8b and EGF are commercially available.

In this cultivation, EpiLCs are seeded to a cellular non-adhesive or low-adhesive culture vessel known per se to obtain a cell density of, for example, about 3 to 10×10⁴ cells/mL, preferably about 4 to 8×10⁴ cells/mL, and cultured in an incubator in an atmosphere of 1-10% CO₂/99-90% air at about 30-40° C., preferably about 37° C., for about 4-10 days, preferably about 4-8 days, more preferably about 4-6 days, more preferably 4 days.

The fact of differentiation into PGC-like cells can be confirmed by, for example, analyzing the expression of Blimp1 by RT-PCR and the like. As required, furthermore, the expression of other genes and cell surface antigens can also be examined. Examples of other genes include Stella. When pluripotent stem cells bearing fluorescent protein genes under the control of Blimp1- and/or Stella-promoters are used as a starting material, the fact of differentiation into PGC-like cells can be confirmed by FACS analysis. When the pluripotent stem cells bear no appropriate transgenic reporter, such as ESCs or iPSCs derived from human or other non-mouse mammals, it is preferable to confirm the fact of differentiation into PGC-like cells by FACS analysis and the like using one or more cell surface antigens specifically expressed on PGC-like cells. As the cell surface antigens, preferably SSEA-1 and integrin-β3 are exemplified.

A cell population containing PGC-like cells derived from pluripotent stem cells and produced by the aforementioned step I) and II) may be a purified population of PGC-like cells, and 1 kind or more of cells other than PGC-like cells may be co-present. Here, “PGC-like cell” is defined as a cell that shows elevated expression of Blimp1 and/or Stella compared to the EpiLC before inducing differentiation, is capable of contributing to normal spermatogenesis, and does not form teratoma when transplanted into an immunodeficient mouse. As stated above, when PGC-like cells are induced using pluripotent stem cells bearing fluorescent protein genes under the control of Blimp1- and/or Stella-promoters as a starting material, the Blimp1- and/or Stella-positive PGC-like cells can be easily isolated and purified by sorting out the cell population obtained in the foregoing step II) using a cell sorter. The PGC-like cells can also be isolated and purified by FACS using a reporter under the control of gene whose expression increases along with Blimp1 and Stella (e.g., Nanog) as a marker.

### 2. Expansion of PGC/PGCLC

In the present step, for example, PGC or PGCLC obtained by the above-mentioned method is cultivated in the presence of a PDE4 inhibitor and/or cyclospoeine A. When the PGCLC to be used is a non-uniform cell population, for example, an SSEA-1 positive and integrin-β3 positive cell fraction can be used by isolating using FACS. As PGCLC, the cells on d4-d10, preferably d4-d8, more preferably d4-d6, further preferably about d4 EpiLC, wherein the day of start of differentiation induction is d0, may be used.

As the medium to be used in the present step, the medium exemplified for the differentiation induction from PSC into EpiLC can be similarly used as the basal medium. It is preferable to add a serum or serum replacement to the medium. The kind and concentration of the serum or serum replacement to be used here may be the same as those exemplified for the differentiation induction from PSC into EpiLC. In addition, the medium may contain other additives known per se. Such additive is not particularly limited as long as it can support the expansion of PGC/PGCLC, and those exemplified for the differentiation induction from PSC into EpiLC can be used in the same manner. Examples of the medium used in this step include, but are not limited to, GMEM medium containing 10% Knockout Serum Replacement (KSR), 2.5% fetal bovine serum (FCS), 0.1 mM NEAA, 1 mM sodium pyruvate, 0.1 mM 2-mercaptoethanol, 100 U/ml penicillin, 0.1 mg/ml streptomycin, 2 mM L-glutamine and the like.

The PDE4 inhibitor to be added to the above-mentioned medium is not particularly limited as long as it is a substance that can inhibit the enzyme activity of PDE4, namely, hydrolysis activity of cAMP. Preferably, it is a selective inhibitor of PDE4 (which does not inhibit not only enzyme other than phosphodiesterase (PDE) but also PDEs other than PDE4). Examples of the inhibitor include, but are not limited to, ibudilast, S-(+)-rolipram, rolipram, GSK256066, cilomilast and the like.

The concentration of the PDE4 inhibitor is, for example, about 0.1 μM or more, preferably about 0.5 μM or more, more preferably about 1 μM or more. The concentration of the PDE4 inhibitor is, for example, about 100 μM or less, preferably about 50 μM or less, more preferably 30 μM or less. In a preferred embodiment, the concentration of the PDE4 inhibitor may be appropriately selected from the range of about 0.5-50 μM, preferably about 1-30 μM.

In the present specification, “Cyclosporine A” includes a cyclic polypeptide consisting of 11 amino acids specified by IUPAC name: cyclo{-[(2S,3R,4R,6E)-3-hydroxy-4-methyl-2-methylaminooct-6-enoyl]-L-2-aminobutanoyl-N-methylglycyl-N-methyl-L-leucyl-L-valyl-N-methyl-L-leucyl-L-alanyl-D-alanyl-N-methyl-L-leucyl-N-methyl-L-leucyl-N-methyl-L-valyl-}, and a derivative thereof known per se (see, for example, WO 2012/051194 and the like). Cyclosporin A can be isolated from the fungus producing same by a fermentation method or can be organically synthesized by a well-known peptide synthesis technique. In addition, a commercially available cyclosporine A (e.g., Sigma-Aldrich Ltd.) can also be used.

The concentration of cyclosporine A is, for example, about 0.1 μM or more, preferably about 0.5 μM or more, more preferably about 1 μM or more. The concentration of cyclosporine A is, for example, about 100 μM or less, preferably about 50 μM or less, more preferably 30 μM or less. In a preferred embodiment, the concentration of cyclosporine A may be appropriately selected from the range of about 0.5-50 μM, preferably about 1-30 μM, more preferably about 1-10 μM.

Preferably, the present culture step is performed using a medium containing at least a PDE4 inhibitor, more preferably, a medium further containing cyclosporine A.

In a preferred embodiment, the present culture step is performed using a medium further containing forskolin.

Forskolin is a potent activator of adenylate cyclase and increases intracellular cAMP levels by a different action mechanism than PDE4 inhibitors. It can act synergistically with PDE4 inhibitors to remarkably increase PGC/PGCLC expansion efficiency.

The concentration of forskolin is, for example, about 0.1 μM or more, preferably about 0.5 μM or more, more preferably about 1 μM or more. The concentration of forskolin is, for example, about 100 μM or less, preferably about 50 pH or less, more preferably 30 μM or less. In a preferred embodiment, the concentration of forskolin may be appropriately selected from the range of about 0.5 to 50 μM, preferably about 1 to 30 μM.

The medium for expansion of PGC/PGCLC preferably further contains SCF. The concentration of SCF is, for example, about 30 ng/ml or more, preferably about 50 ng/ml or more, more preferably about 80 ng/ml or more. The concentration of SCF is, for example, about 200 ng/ml or less, preferably about 150 ng/ml or less, more preferably about 120 ng/ml or less. In a preferred embodiment, the concentration of SCF may be appropriately selected from the range of about 50 to 150 ng/ml, preferably about 80 to 120 ng/ml.

In a particularly preferred embodiment, the medium for expansion of PGC/PGCLC contains 10 μM PDE4 inhibitor, 10 μM forskolin and 100 ng/ml SCF. When combined with other PGC proliferation stimulation factors, dedifferentiation of PGC/PGCLC into EGC may be promoted. Thus, it may be preferable not to add LIE to the medium for expansion of PGC/PGCLC.

In the expansion method of PGC/PGCLC, PGC/PGCLC may be cultured in the presence or absence of feeder cells. The kind of the feeder cell is not particularly limited, and a feeder cell known per se can be used. For example, fibroblasts (mouse embryonic fibroblasts, mouse fibroblast cell line STO and the like) can be mentioned. The feeder cells are preferably inactivated by a method known per se, for example, radiation (gamma rays and the like), treatment with an anticancer agent (mitomycin C and the like) and the like. When the feeder cells are vulnerable to PDE4 inhibitors and/or forskolin, it is desirable to subculture several generations of feeder cells in the presence of these additives and acclimate them to the additives in advance.

The incubator to be used for the expansion of PGC/PGCLC is not particularly limited, and those exemplified in the differentiation induction from PSC into EpiLC can be similarly used.

In this cultivation, PGC/PGCLC is plated onto an incubator (feeder cells seeded thereon in advance) to obtain a cell density of, for example, about 10⁴-10⁵ cells/cm², preferably about 2 to 8×10⁴, and cultured in an incubator under atmospheric conditions of 1-10% CO₂/99-90% air at about 30-40° C., preferably about 37° C., for 3 to 9 days, preferably 4 to 8 days, more preferably 5 to 7 days. As a result of culture, flat colonies are formed, Blimp1 and Stella are strongly expressed, the characteristics of motile cells with filopodia and lamellipodia are shown, and the properties of PGC in the mobile phase are maintained.

From the results of comprehensive gene expression analysis, the amplified PGCLC obtained as mentioned above maintains the gene expression of mobile PGC without increasing the expression of gene group (e.g., Daz1, Ddx4, Piwi12, Mae1, etc.) expressed in late PGC (E12.5 and later).

From the results of epigenetic analysis, amplified PGCLC progressively eliminates 5-methylcytosine in all genomic regions, and faithfully reproduces genome-wide demethylation in the germ cell of the gonad. That is, the amplified PGC/PGCLC obtained by the method of the present invention reproduces the epigenetic blank state of the germ line immediately before sex differentiation.

The amplified PGC/PGCLC obtained by the method (I) of the present invention can be used for varied purposes. For example, since the PGC-like cells transplanted into a testis of a recipient animal can robustly contribute to spermatogenesis in the testis and the generation of healthy offspring, they can be used for the treatment of infertility or hereditary diseases of reproductive tissues.

PGC/PGCLC can be transplanted into a testis by using PGC/PGCLC in place of germlime stem cells (GS cells) in the methods disclosed in WO 2004/092357 and Biol. Reprod., 69: 612-616 (2003). Alternatively, PGC/PGCLC can be cultured in the same manner as in WO 2004/092357 and Biol. Reprod. (2003), supra to induce differentiation into GS cells, then transplanted into a testis.

PGC/PGCLC (including a cell population containing PGC/PGCLC; the same applies below) of the present invention are produced as a parenteral preparation, preferably as an injection, suspension, or drip infusion, in a mixture with a pharmaceutically acceptable carrier, by a conventional means. Examples of the pharmaceutically acceptable carrier that can be contained in the parenteral preparation include aqueous liquids for injection, such as physiological saline and isotonic solutions containing glucose and other auxiliary drugs (e.g., D-sorbitol, D-mannitol, sodium chloride and the like). The agent of the present invention may be formulated with, for example, a buffering agent (e.g., phosphate buffer solution, sodium acetate buffer solution), a soothing agent (e.g., benzalkonium chloride, procaine hydrochloride and the like), a stabilizer (e.g., human serum albumin, polyethylene glycol and the like), a preservative, an anti-oxidant and the like.

When the agent of the present invention is prepared as an aqueous suspension, PGC/PGCLC is suspended in one of the aforementioned aqueous liquids to obtain a cell density of about 1.0×10⁶ to about 1.0×10⁷ cells/ml.

The agent of the present invention can be cryopreserved under conditions typically used for the cryopreservation of stem cells, and thawed immediately before use.

The preparation thus obtained is stable and less toxic, and therefore, it can be safely administered to mammals such as humans. Although the method of administration is not particularly limited, the preparation is preferably administered by injection or drip infusion into a seminiferous tubule. For a male infertility patient, for example, it is usually convenient to administer the agent in an amount of about 1.0×10⁵ to about 1×10⁷ cells, based on the amount of PGC-like cells per dose, once or 2-10 times at about 1- to 2-week intervals.

[II] Induction Method of Oocyte from PGC/PGCLC

The differentiation of amplified PGC/PGCLC obtained by the method (I) of the present invention into oocyte can be induced in the absence of somatic cell of the gonad by culturing same in the presence of BMP and RA. Therefore, the present invention also provides a method for inducing oocyte from PGC or PGCLC by culturing the PGC or PGCLC in the presence of BMP and RA (sometimes to be abbreviated as “the method (II) of the present invention”).

The method (II) of the present invention is characterized in that it is performed in the absence of somatic cell of the gonad. As used herein, the “gonad” refers to a structure consisting of germ cells and somatic cells in support thereof. It is formed by the time when male and female sex differentiation in fetal (pup) primordial germ cells (PGC) begins in the mother' womb (12.5 days after fertilization (E12.5) in mice). PGCs are differentiated into gametes (spermatozoon and ovum) while being surrounded by somatic cells of the gonad which are characteristic of male and female. In the conventional method, somatic cells of the gonad at this time (for example, in the case of mouse, E12.0-E13.0, preferably about E12.5) and PGC/PGCLC are co-cultured to mimic the cellular environment at the time when PGC becomes prespermatogonium or oocyte. In The method (II) of the present invention does not require gonadal somatic cells, and therefore, the operation is not complicated, can perform differentiation into oocytes under specified conditions, and can be used practically for animal species including human where collection of gonadal somatic cells in the embryonic period is difficult.

The PGC/PGCLC to be used in the method (II) of the present invention is not particularly limited as long as at least the gene group important for late PGC and meiosis are demethylated. Since PGC/PGCLC immediately after induction from epiblast and EpiLC has insufficient genomic demethylation, it is preferable to use the amplified PGC/PGCLC obtained by the method (I) of the present invention. For example, PGC/PGCLC cultured in the presence of a PDE4 inhibitor, further preferably forskolin, further more preferably SCF for, for example, 3 days or more, preferably 3 to 9 days, more preferably 3 to 8 days, further preferably 3 to 7 days, can be used.

The medium to be used in the method (II) of the present invention can similarly use, as a basal medium, the medium exemplified for the induction of differentiation of PSC into EpiLC. It is preferable to add a serum or serum replacement to the medium. The kind and concentration of the serum or serum replacement to be used here are the same as those exemplified for the differentiation induction from PSC into EpiLC. The medium may also contain other additives known per se. Such additive is not particularly limited as long as it can support the differentiation from PGC/PGCLC into oocyte, and those exemplified for the differentiation induction from PSC into EpiLC can be used in the same manner. Examples of the medium used in this step include, but are not limited to, GMEM medium containing 10% Knockout Serum Replacement (KSR), 2.5% fetal bovine serum (FCS), 0.1 mM NEAA, 1 mM sodium pyruvate, 0.1 mM 2-mercaptoethanol, 100 U/ml penicillin, 0.1 mg/ml streptomycin, and 2 mM L-glutamine, and the like, like the method (I) of the present invention.

BMP to be added to the above-mentioned medium is not particularly limited as long as it can support differentiation of PGC/PGCLC into oocyte. For example, BMP2, BMP4, BMP5, BMP7 and the like can be mentioned. Preferably, it is BMP2, BMP5 or BMP7. Any one kind of BMP may be used, or two or more kinds of BMP may be used in combination.

The concentration of BMP is, for example, about 100 ng/ml or more, preferably about 200 ng/ml or more, more preferably about 300 ng/ml or more. Also, the concentration of BMP is, for example, about 1,000 ng/ml or less, preferably about 800 ng/ml or less, more preferably about 600 ng/ml or less. In a preferred embodiment, the concentration of BMP may be appropriately selected from the range of about 200 to 800 ng/ml, preferably about 300 to 600 ng/ml.

The concentration of RA is, for example, about 10 nM or more, preferably about 30 nM or more, more preferably about 50 nM or more. The concentration of RA is, for example, about 500 nM or less, preferably about 300 μM or less, more preferably 200 μM or less. In a preferred embodiment, the concentration of RA may be appropriately selected from the range of about 30-300 nM, preferably about 50-200 nM.

In a preferred embodiment, the medium for induction of oocyte from PGC/PGCLC further contains PDE4 inhibitor, forskolin and SCF, like the aforementioned medium for expansion of PGC/PGCLC. The concentration of each additive may be appropriately selected from the same concentration range of the aforementioned method (I) of the present invention.

In a particularly preferred embodiment, the medium for induction of oocyte from PGC/PGCLC contains 500 ng/ml BMP and 100 nM RA.

In the induction method of oocyte from PGC/PGCLC, the PGC/PGCLC may be cultured in the presence or absence of a feeder cell. The kind of the feeder cell is not particularly limited and a feeder cell known per se can be used. The incubator to be used in this culture step is not particularly limited, and those exemplified for the differentiation induction from PSC into EpiLC can be used in the same manner.

In this cultivation, for example, the medium is replaced with a medium supplemented with BMP and RA 3 to 9 days, preferably 3 to 8 days, more preferably 3 to 7 days, after placing PGC/PGCLC under conditions for expansion in the method (I) of the present invention, and culture is continued for further 2-7 days, preferably 2-6 days. As a result of the culture, PGC/PGCLC differentiates synchronously with Daz1-positive, Ddx4-positive, and SCP3-positive oocyte-like cells, and differentiates into the pachytene stage of meiosis.

The present invention is explained in more specifically in the following by referring to Examples. It is needless to say that the present invention is not limited thereto.

### Example

### [I] Expansion of PGC/PGCLC Using PDE4 Inhibitor or the Inhibitor and Forskolin in Combination

For more information on the references cited below, see Ohta, H. et al., EMBO J., 36(13): 1888-1907 (2017).

### <Materials and Methods>

### Mouse

All animal experiments were performed under the ethical guidelines of Kyoto University. BVSC (Acc. No. BV, CDB0460T; SC, CDB0465T: http://www.cdb.riken.jp/arg/TG%20 mutant %20 mice%20 list.html) and Stella-EGFP transgenic mice were established as previously reported (Payer et al, 2006; Seki et al, 2007; Ohinata et al, 2008; Imamura et al, 2010), and maintained mostly in the C57BL/6 background. WBB6F1-W/Wv, C57BL/6, DBA/2, C3H, BDF1, and ICR mice were purchased from SLC (Shizuoka, Japan). The noon of the day when the vaginal plug was confirmed was designated as 0.5 days of embryonic period (E). All mice were housed in a specific pathogen-free animal facility under a 14-hr light/10-hr dark cycle.

### Induction and Culture of ES Cell (ESC)

BVSC R8, H14, and H18 were previously reported (Hayashi et al, 2011, 2012). Female BVSC mice (mostly C57BL/6 background) were mated with male DBA/2 or C3H mice to obtain BDF1 or BCF1 embryos. Blastocysts were seeded and cultured on mouse embryonic fibroblasts (MEF) (Ying et al, 2008; Hayashi et al, 2011) in the wells of a 96-well plate in N2B27 medium containing 2i (PD0325901, 0.4 μM: Stemgent, San Diego, Calif.; CHIR99021, 3 μM: Stemgent) and LIF (1,000 U/ml; Merck Millipore). The proliferated colonies were passaged by dissociating with TrypLE (Thermo Fisher Scientific). ESC was maintained on MEF for up to 2 passages. Thereafter, male ESC was cultured on a dish coated with poly-L-ornithine (0.01%; Sigma) and laminin (10 ng/ml; BD Biosciences) and maintained at feeder free.

### Induction of EpiLC and PGCLC

Induction of EpiLC and PGCLC was performed as reported previously (Hayashi et al, 2011). In short, 1×10⁵ cells of ESC were seeded in N2B27 medium containing activin A (20 ng/ml), bFGF (12 ng/ml), and KSR (1%) in the wells of a 12 well plate coated with human plasma fibronectin (16.7 mg/ml) to induce EpiLC. PGCLC was induced from d2 EpiLC under floating conditions in the presence of cytokine BMP4 (500 ng/ml; R&D Systems), LIF (1,000 U/ml; Merck Millipore), SCF (100 ng/ml; R&D Systems) and EGF (50 ng/ml; R&D Systems) using the wells of a low cell binding U-bottom 96 well plate (Thermo Scientific) in a serum-free medium [GK15 containing 15% KSR; GMEM (Thermo Fisher Scientific), 0.1 mM NEAA, 1 mM sodium pyruvate, 0.1 mM 2-mercaptoethanol, 100 U/ml penicillin, 0.1 mg/ml streptomycin, and 2 mM L-glutamine]. To prepare many PGCLCs for compound library screening, PGCLC was induced with AgglreWell400 (STEMCELL Technologies) using the same medium.

### Fluorescence Activated Cell Sorting

Sample preparation from cell aggregates was performed as previously reported (Hayashi et al, 2011). FACS was performed on a FACSAriaIII (BD) cell sorter. BV and SC fluorescence was detected on FITC and AmCyan Horizon V500 channels, respectively. Data was analyzed using FACSDiva (BD) software.

Establishment of m220 Subline

The m220 cell line (Majumdar et al, 1994) was cultured in DMEM containing 10% FCS on a plate coated with gelatin. Since the m220 cells were very vulnerable to a mitomycin C (MMC) treatment, m220 subline resistant to MMC was established. In short, by FACS, single m220 cells were seeded on the wells of a 96 well plate (6 plates). One week after the seeding, cell proliferation was observed in about half of the wells. The cells were passaged in one well each of two 96 well plates, one plate was frozen as a replica and the other plate was treated with MMC (4 μg/ml, for 2 hr). On day 10 from the MMC treatment, MMC resistance was evaluated by microscopic observation. A total of 242 m220 sublines were established, and 7 sublines showed high MMC resistance. The m220-5 sublines were mainly used for the experiment.

### Detection of BV(+) PGCLC by Cell Analyzer

d4 PGCLC was seeded by FACS on m220-5 feeders in 96-well plates and BV fluorescence was monitored by cell analyzer (Cellavista; SynenTec). Fluorescence photograph for BV was taken by Cellavista cell analyzer with the following settings: 10× objectives; exposure time: 140 μsec; gain: 4×; binning: 4×4; excitation: 500/24 nm; emission: 542/27 nm. BV fluorescence was detected using the following algorithm/attribute parameters: sensitivity: 10; region merging: 200; min. granule intensity: 50; well edge distance: 200; contrast: 1; size: 3,000; intensity: 255; roughness: 500; granularity: 100; granule intensity: 255; granule count: 10,000; longishness: 100; compactness: 1. The value of “cell nucleus” was used for the detection of BV fluorescence.

### Compound Library Screening for PGCLC Proliferation

The compound library was screened at concentrations of 10 μM and 1 μM. A 96-well plate containing m220-5 cells treated with MMC was used. In each 96-well plate, negative (DMSO only) and positive (LIF) controls were assigned to both sides and a compound was added to 80 wells. 200 BV (+) d4 PGCLCs induced from BDF1-2 ESC were seeded in the wells of a 96-well plate, and BV fluorescence was measured on the Cellavista cell analyzer on day 1 of culture (c1), c3, c5 and c7. The value of “cell nucleus” was used for the detection of BV fluorescence. Since the proliferation rate of d4 PGCLC was slightly different between experiments, the values from different experiments were adjusted based on the mean value of the negative control obtained from the first experiment. For each compound, the fold difference in BV fluorescence between c1 and c7 was calculated for each compound and compounds with a fold difference value exceeding 3SD of the mean of the negative control were identified as those that potentiate PGCLC proliferation.

### Expansion Culture of d4 PGCLC and E9.5 PGC

Since the m220-5 feeder treated with MMC was vulnerable to forskolin and rolipram, m220-5 cells were subcultured 3 times with both forskolin and rolipram each at 10 μM and adapted to these compounds before MMC treatment (1-2 μg/ml for 2 hr). d4 PGCLC or E9.5 PGC(BDF1×Stella-EGFP) was selected by FACS, and the cells were seeded on m220-5 cells in GMEM medium containing 10% KSR, 0.1 mM NEAA, 1 mM sodium pyruvate, 0.1 mM 2-mercaptoethanol, 100 U/ml penicillin, 0.1 mg/ml streptomycin, 2 mM L-glutamine, 2.5% FCS, 100 ng/ml SCF, 10 μM forskolin, and 10 μM rolipram. Half of the culture medium was changed every 2 days.

### Immunofluorescence

The following antibodies were used as the indicated dilution solutions: rabbit anti-MVH (1/250; Abeam ab13840); rabbit anti-DAZL (1/250; Abeam ab34139); mouse anti-OCT4 (1/250; BD 611203); mouse anti-5 mC (1/500; Abeam ab10805); rabbit anti-H3K27 me3 (1/500; Millipore 07-449); rabbit anti-H3K9 me2 (1/500; Millipore 07-441); rabbit anti-DNMT1 (1/100; Santa Cruz Biotechnology sc-20701); mouse anti-DNMT3A (1/200; Abeam ab13888); mouse anti-DNMT3B (1/200; Novus Biologicals NB100-56514); rabbit anti-UHRF1 (1/100; Santa Cruz Biotechnology sc-98817); and chicken anti-GFP (1/500; Abeam ab13970). The following secondary antibodies obtained from Thermo Fisher Scientific were used at 1/500 dilution: Alexa Fluor 568 goat anti-rabbit IgG; Alexa Fluor 568 goat anti-mouse IgG; Alexa Fluor 488 goat anti-chicken IgG. Phalloidin (1/40, Thermo Fisher Scientific A12380) conjugated with Alexa Fluor 568 was used to stain F-actin.

A protocol for immunofluorescence staining has been previously reported (Hayashi et al, 2011; Nakaki et al, 2013). For MVH, DAZL and OCT4 staining, d4c7 PGCLC (BDF1-2) was selected by FACS, mixed with male PGC at E13.5 at a ratio of 1:1, and spread on glass slides coated with MAS by using Cyto Spin 4 (Thermo Fisher Scientific). E13.5 male germ cells (ICR) were sorted by FACS using SSEA1 antibody conjugated with Alexa Fluor 647. For 5 mC, H3K27 me3 and H3K9 me2 staining, D4c7 PGCLC (BDF1-2) was selected by FACS, mixed with d2 EpiLC in a 1:1 ratio and spread on glass slides coated with MAS by using Cyto Spin 4 (Thermo Fisher Scientific). Images were captured with a confocal microscope (Zeiss, LSM780) and signal intensity was analyzed by ImageJ (NIH).

Measurement of cAMP Concentration

The intracellular cAMP concentration was measured using cAMPGlo Max assay kit (Promega) according to the manufacturer's instructions. A standard curve using purified cAMP was prepared by calculating the Δrelative light unit (ΔRLU) (RLU [0 nM]-RLU [X nM]). For each sample, 1×10⁴ d4 PGCLC was pretreated with forskolin and/or rolipram for 30 min at room temperature and the ΔRLU (RLU [untreated sample-RLU [treated sample]) was calculated. An increase in the intracellular cAMP level due to chemical treatments was inferred from the cAMP standard curve. Three biological replicates were analyzed for each sample.

### Cell Cycle Analysis

The cell cycle status of ESC, EpiLC, d4, d4c3, d4c5 and d4c7 PGCLC(BDF1-2) at E13.5, E14.5 and E15.5 and male germ cells was examined in the same manner as in a previous report (Kagiwada et al, 2013). To label the cultured cells, the cells were incubated with BrdU (10 μM) for 30 min. To label gem cells, female mice (ICR) were mated with Stella-EGFP males and pregnant females were intraperitoneally injected with 1 mg BrdU and embryos were harvested 30 min later. Cultured cells or male gonads were dispersed in single cells by TrypLE treatment. To detect BrdU uptake, the APC-BrdU Flow Kit (BD Biosciences) was used according to the manufacturer's instructions. The stained samples were analyzed using BD FACSAriaIII (BD) with FACS Diva (BD) software, and PGCLC or male gem cells were respectively identified by BV or Stella-EGFP fluorescence. Three biological replicates were analyzed for each sample.

Transplantation of PGCLC into Testis of W/W^{v} Mouse

After purification of PGCLC by FACS, 1×10⁴-1×10⁵ cells per testis were injected into the testes of randomly selected neonatal (7 days old) or adult W/W^{v} mouse, as previously reported (Chuma et al, 2005). Where necessary, anti-mouse CD4 antibody (50 mg per dose, clone GK1.5; Biolegend) was injected intraperitoneally on day 0, 2 or 4 for immunosuppression (Kanatsu-Shinohara et al, 2003). To assess pregnancy, recipients at 10 weeks post-transplant were mated with BDF1 females. The genotype of BVSC transgene progeny was determined as previously reported (Ohinata et al, 2008). For HE staining, testis or epididymis was fixed with Bouin's solution and embedded in paraffin, and sections were produced.

### In Vitro Fertilization

Spermatozoons were collected from the cauda epididymis and pre-incubated in HTF medium (Kyudo Co., Ltd.) for 1 hr at 37° C. Eggs were collected from BDF1 females that were superovulated by injecting PMSG and hCG and fertilized with sperm in HTF medium. The obtained 2-cell embryos were transferred to the fallopian tube of pseudopregnant ICR females 0.5 days after pregnancy (dpc). Pups were delivered by cesarean section at 18.5 dpc.

### LacZ Staining

The seminiferous tubes were fixed with 2% paraformaldehyde and 0.2% glutaraldehyde in PBS at 4° C. for 1 hr. After washing 3 times with PBS, the seminiferous tubes were washed with X-gal solution (PBS containing 0.1% X-gal, 0.1% Triton X-100, 1 mM MgCl₂, 3 mM IQ [Fe(CN)₆] and 3 mM K₃ [Fe(CN)₆] and incubated at 37° C. for 2-3 hr.

### DNA FISH and Immunofluorescence-DNA FISH in PGCLC

ESCs, EpiLCs, and female MEFs were dissociated with TrypLE, and d4, d4c3 and d4c7 PGCLCs were purified using FACS. Cell samples were transferred onto poly-L-lysine (Sigma)-coated glass coverslips in a small amount of PBS and adhered to the coverslips by absorbing excess medium before fixation. For DNA FISH, the cell samples on the coverslips were fixed in 3% paraformaldehyde (PFA) (pH7.4) for 10 min, permeabilized in 0.5% Triton X-100/PBS for 3 min on ice, and stored in 70% ethanol at −20° C. After dehydration with a dilution series of ethanol, the samples were denatured in 70% formamide/2×SSC (pH 7.4) at 80° C. for 30 min, and dehydrated again with an ice-cold ethanol series. Then, the samples were hybridized with a fluorescent BAC probe for Huwe1 (RP24-157H12) at 37° C. overnight. Coverslips were counterstained with DAPI (1 μg/ml) and mounted on Vectashield (Vector Laboratories).

Immunofluorescence followed by DNA FISH was performed as described previously (Chaumeil et al, 2004, 2008). The cell samples on the coverslips were fixed in 3% PFA (pH 7.4) for 10 min at room temperature. The cells were permeabilized on ice for 3 min in 0.5% Triton X-100/PBS. After washing with PBS, the prepared products were blocked in 1% BSA (Sigma)/PBS for 30 min, incubated with anti-H3K27me3 (1/200; Millipore) overnight at 4° C., then washed 3 times with PBS, and incubated with Alexa Fluor 488 anti-rabbit secondary antibody (1/500; Thermo Fisher Scientific) for 30 min at room temperature. After washing in PBS, the prepared products were fixed in 4% PFA for 10 min at room temperature and then washed with PBS. The prepared products were incubated in 0.7% Triton X-100, 0.1 M HCl for 10 min on ice. They were then washed twice in 2×SSC for 10 min at room temperature. Finally, the prepared product were denatured in 70% formamide/2×SSC (pH7.4) for 30 min at 80° C., immersed in ice-cold 2×SSC and hybridized with the above-mentioned fluorescent BAC probe for Huwe1.

### RNA Sequencing (RNA-Seq)

Using RNAeasy Micro Kit (Qiagen), total RNA was purified from d4, d6, d4c3, d4c5, and d4c7 PGCLC (two biological replicates each) of ESC, EpiLC and BV and SC double positive (sometimes to be abbreviated as “BVSC(+)” in the present specification). As reported previously (Kurimoto et al, 2006), ng RNA (corresponding to 1,000 cells) was subjected to cDNA replication method, and as reported previously (Nakamura et al, 2015), the 3′-terminal was subjected to deep sequencing by the SOLiD 5500×1 system. Male/female germ cell-derived 1 ng RNA at j E10.5 PGC, E12.5 and E13.5 prepared in a previous study (Kagiwada et al, 2013), and cDNAs amplified from E9.5 PGC (two biological replicates each) were also subjected to RNA-seq.

### Chromatin Immunoprecipitation Sequence (ChIP-Seq)

ChIP-seq was performed as reported previously (Kurimoto et al, 2015). In short, 1×10⁵-1×10⁶ BV(+)d4c7 PGCLC were purified by FACS and fixed with 1% formalin (Sigma) for 10 min at room temperature and then quenched with 150 mM glycine. The fixed cells were washed with PBS, lysed in a lysis buffer containing 1% SDS, and sonicated with Bioruptor UCD250 for 10 cycles with a high power for 30 sec. The solubilized chromatin fraction was incubated with M280 Dynabeads sheep anti-mouse IgG (Life Technologies) and a mouse monoclonal antibody against histone H3K4me3, H3K27ac, or H3K27me3 (Hayashi-Takanaka et al., 2011) in the complex while rotating overnight at 4° C. (two biological replicates each). After washing, chromatin was eluted in a buffer containing 1% SDS and 10 mM DTT. The eluate was reverse-crosslinked overnight at 65° C., treated with 4 μg of proteinase K at 45° C. for 1 hr, and purified by Qiaquick PCR purification column (Qiagen). The ChIP-treated DNA and input DNA were then sheared by sonication (Covaris, Woburn, Mass.) to an average size of about 150 bp and subjected to a library preparation method (Kurimoto et al., 2015) for deep sequencing in the SOLiD5500×1 system in the previous report (Kurimoto et al, 2015).

### Bisulfite Sequencing of Whole Genome (WGBS)

WGBS was performed as reported previously (Shirane et al, 2016). In short, BV positive d4c3 and d4c7 PGCLCs (two biological replicates each) purified by 10 mM tris-Cl (pH 8.0) containing 150 mM NaCl, 10 mM EDTA, 0.5% SDS and 1 mg/mL proteinase K were thawed at 55° C. with shaking overnight. The lysate was incubated with 1.32 μg/ml RNase A at 37° C. for 1 hr, and extracted once with TE-saturated phenol, twice with phenol-chloroform, and once with chloroform. Genomic DNA was precipitated with an equal volume of isopropanol, washed twice with 70% ethanol, air-dried, and then dissolved in 10 mM Tris-Cl (pH 8.0). Purified genomic DNA (50 ng) was spiked with 0.5 ng unmethylated lambda phage DNA (Promega), and subjected to bisulfite conversion and library construction using post-bisulfite adaptor tagging (PBAT) method (Miura et al, 2012) for deep sequencing on the Illumina HiSeq 1500/2500 system as reported previously (Shirane et al, 2016).

### Data Analysis of RNA-Seq

As described previously (Nakamura et al, 2015), RNA-seq read data was mapped onto the mouse mm10 genome by using cutadapt v1.3 (Martin, 2011), tophat v1.4.1/bowtie v1.0.1 (Kim et al, 2013), and cufflinks v2.2.0 (Trapnell et al, 2012), and annotated to a reference gene with the terminal region of the extended transcript. Expression levels were normalized to reads per million-mapped reads (RPM). Significant expression levels were defined as log₂ (RPM+1)>3. Genes were considered to be expressed differentially when the fold changes in the expression level were greater than 2 (i.e., the difference in log₂ (RPM+1) was greater than 1). Genes that were significantly expressed in at least one sample and were differentially expressed in at least one pair of comparison (10,437 genes) were used for principal component analysis (PGA) and unsupervised hierarchical clustering (UHC). Gene ontology (GO) of differentially expressed genes (Ashburner et al, 2000) was analyzed using the DAVID program (Huang da et al, 2009).

### Data Analysis of ChIP-Seq

As reported previously (Kurimoto et al, 2015), read data of ESC, EpiLC, d6 PGCLC (Kurimoto et al, 2015), and d4c7 PGCLC was mapped onto the mouse mm10 genome by using bowtie v1.1.2 (Langmead et al, 2009), picard-tools v2.1.0 (http://broadinstitute.github.io/picard/), IGVtools v2.3.52 (Robinson et al, 2011), samtools v1.3 (Li et al, 2009), and MACS v2.1.0 (Zhang et al, 2008), and analyzed. Read pattern was visualized by IGV (Robinson et al, 2011).

H3K4me3 peaks with P values of less than 10⁻⁵ and detected in close proximity (within 1 kb) were combined as a single peak, and read densities of the peaks within 500 bp from the center were normalized to those of Input (500 bp or more and within 5 kb thereof) (IP/input level). The H3K4me3 peak with the highest IP/input level located within 2 kb of TSS was considered as the peak associated with TSS. The IP/input level of the H3K4me3 peak associated with TSS was further normalized to that relating to genes with 95 percentile of the significant expression level and defined as the H3K4me3 level.

The H3K27ac peak with a P value smaller than 10⁻²⁰ detected in proximity (within 1 kb) was combined as a single peak. The read densities of peaks within 500 bp from the center were normalized to the mean of log₂ IP/input levels and defined as H3K27ac levels. When the fold change in H3K27ac level was greater than 2, the H3K27ac peak was considered to be biased to d6- or d4c7.

The read densities of TSS (within 1 kb) and H3K27me3 in the region around the H3K4me3 peak and related to TSS were normalized by Input, and to define H3K27me3, normalized to the mean of IP/input levels of H3K27me3 around the TSS of a gene having an expression level with log₂ (RPM+1) greater than 2.5 and smaller than 3.5.

### Data Analysis of WGBS

Adapter trimming, mapping to mouse mm10 genome, and analysis of WGBS data were performed using Trim Galore! v0.4.1 (http://www.bioinformatics.babraham.ac.uk/projects/trim_galore/), cutadapt v1.9.1, and Bismark v0.15.0 (Krueger & Andrews, 2011), bowtie v1.1.2 and R program, as in the previous report (Shirane et al, 2016). The bisulfite conversion rate was estimated to be >99.5% using the lambda phage genome as a positive control.

As reported previously (HCP, ICP and LCP) (Borgel et al, 2010), the promoter was defined as the region 0.9 kb upstream and 0.4 kb downstream from the transcription start site, and classified into 3 categories according to the GC content and CpG density. Promoters with at least 5 CpGs were used for methylation analysis. The ICR coordinates defined in the E12.5 embryo were obtained from a previous publication (Tomizawa et al, 2011). For methylation analysis of repeat factors, the processed reads were mapped on repeat consensus sequence (Shirane et al, 2016) and CpG sites covering at least 4 reads were used. RepeatMasker (Smit, A F A, Hubley, R&Green, P. Repeat-Masker Open-4.0. 2013-2015 http://www.repeatmasker.org) was used to define uniquely mapped region that overlaps with repeat. For analysis of uniquely mapped whole genomic regions, 5mC levels in a 2 kb sliding window with 1 kb overlap were calculated.

For methylation analysis of uniquely mapped regions, CpG sites covering less than 4 reads and more than 200 reads were excluded. Therefore, the minimum sequence depth for calling methylated/unmethylated cytosine was 4. For methylation analysis of repeat factors, the processed reads were mapped on repetitive consensus sequences (RepBase19.0.4) and CpG sites covering at least 5 reads were used.

### Accession No.

The accession number for RNA sequence data for d4c3/d4c5/d4c PGCLC and E9.5/E12.5 germ cells is GSE87644 (GEO database). RNA sequence data for ESC/EpiLC/d4/d6 PGCLC [BVSC (+)] (GSE67259) and E10.5/E11.5/E13.5 germ cells (GSE74094) were downloaded from the GEO database. The accession number for the ChIP sequence data of H3K4me3, H3K27ac, and H3K27me3 of d4c7 PGCLC is GSE87645 (GEO database).

ChIP sequence data of H3K4me3, H3K27ac, H3K27me3 of ESC/EpiLC/d2/d4/d6 PGCLC (GSE60204) was downloaded from the GEO database. The accession number of the WGBS sequence data of d4c3/d4c7 PGCLC is DRA005166 (DDBJ database). WGBS sequence data of ESC/EpiLC/d2/d4/d6 PGCLC (DRA003471) and E10.5/E13.5 PGC (DRA000607) were downloaded from the DDBJ database.

### <Results>

Screening for Compound that Amplifies PGCLC

Male stem cell lines of some novel mouse embryonic stem cells (ESC) having Blimp1-mVenus and Stella-ECFP (Blimp1-mVenus is also called BV, Stella-ECFP is also called SC) transgene (Ohinata et al, 2008) were obtained, and the efficiency thereof in induction and proliferation of PGCLC was evaluated. All ESC lines were induced into EpiLC by activin A (ActA) and basic fibroblast growth factor (bFGF) and induced by bone morphogenetic protein 4 (BMP4), leukemia inhibitory factor (LIF), stem cell factor (SCF), and epidermal growth factor (EGF) into BV positive or BV and SC double positive (sometimes abbreviated as “BV/BVSC (+)” in the present specification) PGCLC. The number of BV(+) PGCLC in floating aggregates increased until day 6 (d6) or day 8 (d8) of induction and then decreased. This is in agreement with a previous report of the present inventors (Hayashi et al, 2011). Of the strains evaluated, BVSC BDF1-2 showed the strongest induction and proliferation in floating aggregates. This strain was used for subsequent screening.

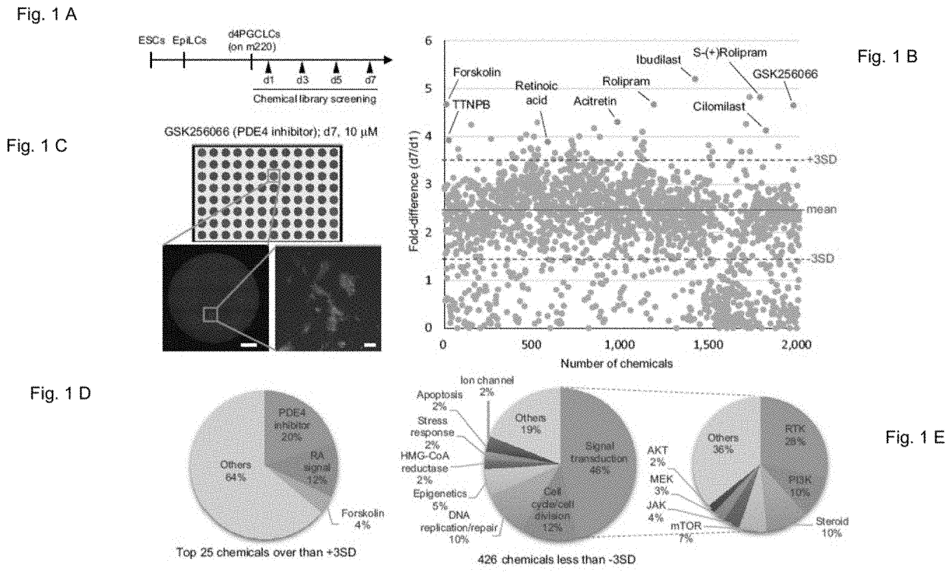

d4 PGCLC seemingly in a proliferation period in floating aggregates was seeded on a 96-well plate together with m220 feeder (Dolci et al, 1991; Majumdar et al, 1994) expressing a membrane-bound SCF known to support the survival of PGC. In addition, it was decided to screen for compounds that enhance the proliferation of BV(+) PGCLC by using a cell analyzer (FIG. 1A). Since ESC and EGC do not or only slightly express Blimp1 or BV (Durcova-Hills et al, 2008; Ohinata et al, 2008; Hayashi et al, 2011), BV positivity was assumed to distinguish PGCLC proliferation from PGCLC dedifferentiation into embryonic germ cell (EGG) (Matsui et al, 1992). The m220 cells were very vulnerable to mitomycin C (MMC) treatment, which is required for the preparation thereof as a feeder. Thus, an m220 subline resistant to MMC treatment was cloned and used. Using LIE (Matsui et al, 1991), which is a classical factor known to stimulate PGC proliferation under selected conditions, PGCLC proliferation was successfully monitored by a cell analyzer as a corresponding increase in BV fluorescence after 7 days of culture. Under this simple positive control condition, it was confirmed that there was little or no dedifferentiation of PGCLC into EGC. Therefore, a total of about 2,000 compounds targeting various sets of intracellular signal transduction molecules/pathways were screened for as to the ability to amplify BV(+)d4 PGCLC after 7 days of culture. As a result, at a concentration of 10 μM, 63 compounds were identified that significantly expanded BV(+) cells compared to negative control culture. The fold difference in BV fluorescence between day 1 and day 7 of culture was greater than 3 SD (standard deviation) of the mean of negative control (FIGS. 1B and C). In particular, of the top 25 hit compounds, five (20%) were selective inhibitors against phosphodiesterase 4 (PDE4) [ibudilast, S-(+)-rolipram, rolipram, GSK256066, cilomilast], three (12%) were agonists (Acitretin, TTNPB, retinoic acid) for retinoic acid (RA) signal transduction, and one was forskolin (FIG. 1D). PDE4 catalyzes the hydrolysis of cyclic AMP (cAMP) to AMP, and PDE4 inhibitors increase intracellular cAMP levels (Pierre et al., 2009; Keravis&Lugnier, 2012). Forskolin is a potent activator of adenylate cyclase and therefore also increases intracellular cAMP levels (Pierre et al, 2009). RA signaling and forskolin are known to stimulate PGC proliferation (De Felici et al, 1993; Koshimizu et al, 1995). Other selective inhibitors of PDE or non-selective inhibitors of PDE did not show a positive effect on PGCLC proliferation. FIG. 1C shows proliferation of BV(+) cells in the presence of the PDE4 inhibitor, GSK256066, on day 7 of culture, revealing the formation of plural colonies with unique flat morphology. A similar screening at 1 μM concentration using some of the same compound libraries identified the same class of compounds (selective inhibitor of PDE4, agonist of RA signaling, and forskolin) as potent stimulants of PGCLC proliferation.

In addition, 426 and 178 compounds negatively affecting the proliferation or survival of BV(+) cells were respectively identified from the 10 and 1 μM screening (fold reduction of BV fluorescence between days 1 and 7 of culture was greater than the negative control mean 3SD: FIGS. 1B and E). Such compounds include inhibitors of major signal transduction pathways, and inhibitors for cell cycle/cell division and DNA replication/repair, including those known to have a positive effect on PGC proliferation/survival, such as pathways for receptor tyrosine kinase (RTK) signal transduction, phosphatidyl inositol 3 kinase (PI3K) signal transduction, mammal rapamycin target (mTOR) signal transduction, Janus kinase (JAK) signal transduction, and AKT signal transduction [reviewed in (Saitou & Yamaji, 2012)]. Taken together, these findings strongly indicate that the screening has successfully identified compounds that affect key pathways relating to PGC proliferation/survival.

### Synergistic Effect of Rolipram and Forskolin on PGCLC Expansion

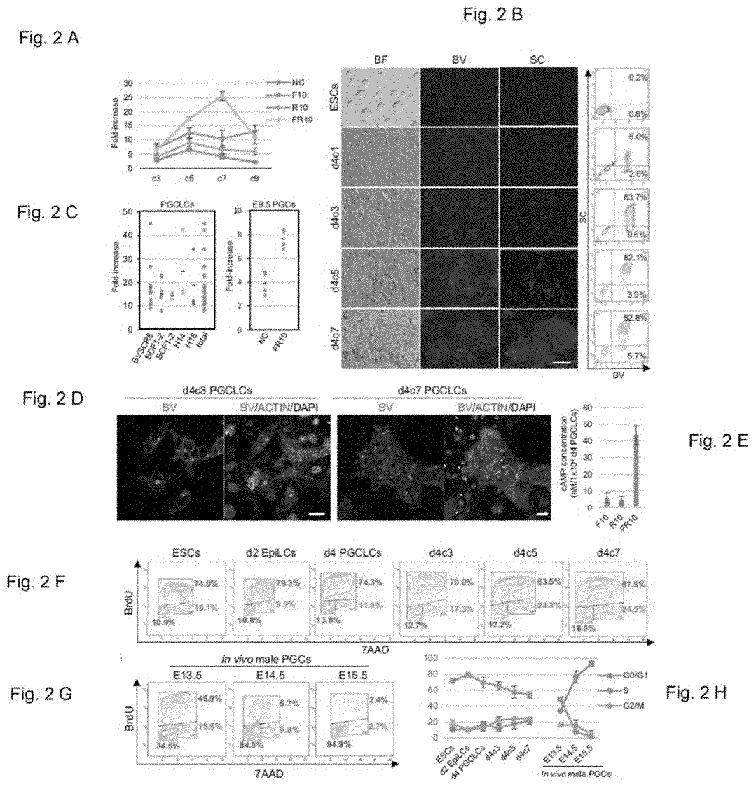

Of the hit compounds, the PDE4 inhibitor was the most concentrated compound (FIG. 1D). In subsequent studies, the effect of one of the PDE4 inhibitors, rolipram, was focused on. Rolipram has been used reproducibly in various experiments as an efficient PDE4 inhibitor (Keravis&Lugnier, 2012). The effect of rolipram, forskolin and their combination (both increase intracellular cAMP concentration by different mechanisms) (Pierre et al, 2009) on the proliferation of d4 PGCLC induced from BVSC BDF1-2 ESC in GMEM/10% KSR/2.5% FCS in the presence of SCF on m220 feeder was evaluated. LIF may enhance PGCLC dedifferentiation into EGG when applied with other stimulation factors of PGC proliferation (Matsui et al, 1992), and thus excluded from the culture. The effect of rolipram alone (10 μM) was relatively mild and similar to that of forskolin alone (10 μM) (FIG. 2A). However, the combination of rolipram and forskolin effectively stimulated proliferation of d4 PGCLC: with both rolipram and forskolin at 10 μM (FR10), d4 PGCLC showed at least highly stable proliferation until day 7 of culture (d4c7), and increased 20-fold or more which corresponds to 4 to 5 doublings (FIG. 2A-C). Importantly, the expanded cells formed flat colonies, continued to strongly express BVSC, and showed the characteristics of motor cells accompanied by prominent flopodium and flagellum adenoma (FIGS. 2B and D). It is thus suggested that the properties of mobile phase PGC are maintained after expansion with FR10.

To investigate whether forskolin and rolipram actually increased cAMP concentration in PGCLC, an increase in cAMP concentration in PGCLC in response to forskolin, rolipram, or both was measured. As shown in FIG. 2E, forskolin and rolipram independently increased the cAMP concentration in PGCLC to about 4 nM/1×10⁴ d4 PGCLC. More notably, simultaneous addition of forskolin and rolipram (FR10) increased the cAMP concentration in PGCLC to more than about 40 nM/1×10⁴ d4 PGCLC.

FR10 was effective in amplifying PGCLC derived from other male and female ESC strains at an average expansion rate of about 20 times on day 7 of culture. In some cases, PGCLC was amplified about 50-fold, corresponding to 5-6 doublings (FIG. 2C). FR10 was also effective in amplifying PGC at E9.5, but to a somewhat limited extent (up to about 8-fold expansion, FIG. 2C), which may be due to the difference in viability under current conditions between E9.5 PGC isolated directly from embryo and d4 PGC derived from PSC in vitro. Cell cycle analysis showed that the majority (>about 60%) of cultured PGCLC were in S phase with a slight decrease in S and G2/M phases and a clear corresponding increase on day 7 of culture (FIGS. 2F and H). The proliferation discontinues in the G0/G1 phase after E13.5, and this is in sharp contrast to the cell cycle characteristics of male germ cells in the obstructed embryo gonad (Western et al, 2008) (FIGS. 2G and H). These findings indicate that rolipram and forskolin act synergistically to advance the cell cycle of PGCLC, presumably through robust activation of cAMP signal transduction.

### Potent Ability of Spermatogenesis of Expansion Cultured PGCLC

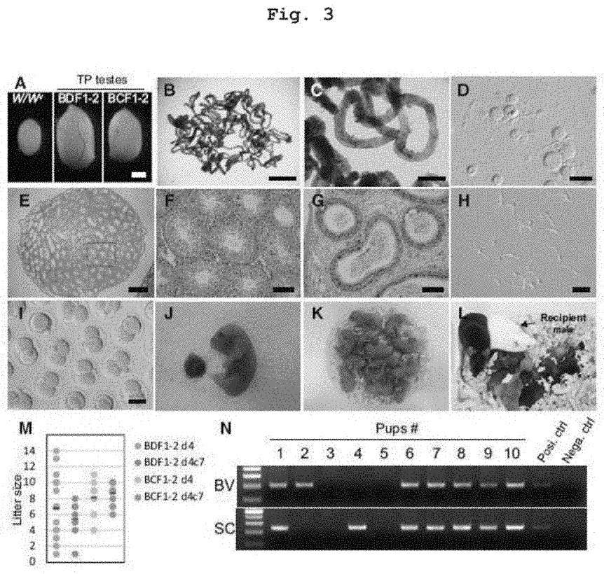

Next, it was evaluated whether PGCLC amplified by FR10 maintained the function as PGC/PGCLC during culture. To this end, d4c7 and d4 PGCLC induced from BVSC BDF1-2, BCF1-2, or R8 (mainly C57BL/6) ESCs were transplanted into the testes of neonatal W/Wv mice lacking endogenous germ cells. Testes transplanted with d4c7 PGCLC and d4 PGCLC derived from BVSC BDF1-2 or BCF1-2 ESC showed a marked increase in size 7 months after transplantation (FIG. 3A), contained many seminiferous tubule with evidence of spermatogenesis and actually produced abundant spermatozoon (FIG. 3B-F). Notably, restoration of spermatogenesis by both d4c7 and d4 PGCLC derived from BVSC BDF1-2 or BCF1-2 ESC became so prominent that spermatozoon could be transported to the cauda epididymis, and such spermatozoon clearly acquired the robust motility used for in vitro fertilization (IVF) to produce normal offspring (FIG. 3G-K). Therefore, recipient males of BVSC BDF1-2 or BCF1-2 ESC-derived d4c7 and d4 PGCLCs were able to produce substantially litter-sized offspring by natural mating, and the resulting offspring clearly showed normal growth (FIG. 3 L-N).

In contrast, testes transplanted with BVSC R8 ESC-derived d4c7 or d4 PGCLC showed only a modest increase in size, reduced number of seminiferous tubules with spermatogenesis, and the resulting spermatozoon did not reach cauda epididymis. Nevertheless, as previously reported (Hayashi et al, 2011), intracytoplasmic injection (ICSI) of the resulting spermatozoon was able to obtain clearly normal offspring. Importantly, as in the case of d4/d6 PGCLC or PGC (Ohta et al., 2004), d4c7 PGCLC derived from either ESC strain failed to colonize adult testes. Teratoma formation was not observed in any of the transplants. Taken together, these findings show that PGCLC amplified by FR10 during culture faithfully maintain its original functional properties, and PGCLC with a hybrid genetic background is sufficiently strong to reconstitute spermatogenesis in infertile mice. Thus, the recipient mice produce offspring by natural mating.

### Transcription Property of PGCLC in Expansion Culture

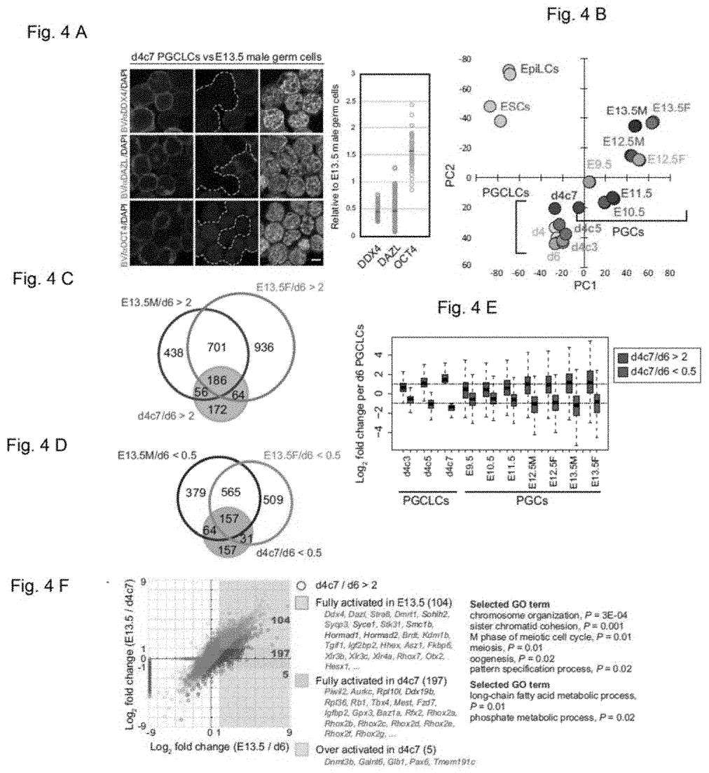

Next, detailed transcriptional properties of PGCLC in expansion culture were determined. First, immunofluorescence (IF) analysis showed that d4c7 PGCLC expressed higher levels of OCT4 compared to E13.5 male germ cells, whereas PGC expressed lower levels of DDX4 and DAZL that are major translation regulatory factors that gradually upregulate after embryonic gonadal colonization (Fujiwara et al., 1994; Cooke et al., 1996) (some d4c7 PGCLCs fully express DAZL) (FIG. 4A). Second, the RNA sequencing (RNA-seq) method (Nakamura et al, 2015, 2016) was used to determine the transcripts of cultured PGCLC [d4c3, d4c5 and d4c7 PGCLC induced from BVC R8 ESC], and they were compared to the transcripts of ESC, EpiLC, d4/6 PGCLC, and germ cell [PGCs at E9.5, E10.5 and E11.5; male/female germ cells at E12.5 and E13.5 (Kagiwada et al, 2013)]. Notably, the main component analysis (PCA) revealed that cultured PGCLC clusterized with d4/6PGCLC and then with E9.5, E10.5 and E11.5 PGCs nearby but far from E12.5 and E13.5 male/female germ cells (FIG. 4B), indicating that PGCLC completely maintains transcriptome during its expansion culture. On the other hand, PCA between unsupervised hierarchical clustering (UHC) and PGCLC revealed a gradual transition of the properties of PGCLCs from d4 to d6 and then to d4c3, d4c5, and d4c7 PGCLC. Therefore, PGCLC appears to undergo directional transcriptional change during expansion culture, and its early stage also appears in floating aggregates. A differentially expressed gene (DEG) was identified between d4c7 and d6 PGCLC, and E13.5 and d6 PGCLC male/female germ cells. d4c7 PGCLC up/downregulates 478 and 409 genes, respectively, and the upregulated genes were enriched by those having gene ontology (GO) functional terms such as “intracellular signaling cascade” and “pattern specific process”. Consistent with PCA, the DEG between E13.5 and d6 PGCLC male/female germ cells was much higher in number (FIGS. 4C and D). E13.5 male/female germ cells up/down regulated the genes of 2,381 and 1,705, respectively, and these DEGs showed enrichment of GO terms that reflected major developmental progression during germ cell development. For example, genes specifically upregulated in males were enriched in “transcription” (Foxo1, Utf1, Pou6f1) and “chromatin composition” (Ezh1, Prmt5, Kdm2a); genes specifically upregulated in females were enriched in “regulation of transcription” (Gata2, Msx1, Cdx2) and “gamete generation” (Fig1a, Nr6a1, Rec8); especially, genes generally upregulated in both males and females were enriched in “meiosis” (Spol1, Mae1, Sycp1), “chromosomal organization” (Ehmt1, Suv39h1, Smarcc1), and “methylation” (Piwi14, Satb1), and therefore, they were enriched for so-called “germline genes” which are previously identified as genes involved in germline functions such as meiosis and transposon suppression and are suppressed in somatic cells mainly by DNA methylation.

Consistent with UHC and PCA between PGCLCs, DEG between d4c7 and d6 PGCLCs progressively acquired such a state in culture (FIG. 4E) and was progressive during embryonic cell development, especially in vivo, and also showed progressive up/down regulation during germ cell development (FIG. 4E). They constituted a minor part, the majority of which was contained in the DEG between male/female germ cells in E13.5 and d6 PGCLC (FIGS. 4C and D). Importantly, they showed no bias for sex-specific regulation (FIGS. 4C and D). To obtain a more detailed insight into the relationship between DEG between d4c7 PGCLC and d6 PGCLC, and DEG between male/female germ cells at E13.5 and d6 PGCLC, the difference in the expression level between the male/female germ cells at E13.5 and d6 PGCLC was j plotted against difference in the expression level between female/female germ cells at E13.5 and d6 PGCLC (FIG. 4F). This analysis clarified that only partially activated 104 of the 306 genes that were generally upregulated in male/female germ cells at E13.5 and d4c7 PGCLC compared to d6 PGCLC (E13.5-d4c7>2-fold), and 197 genes were completely activated in d4c7 PGCLC (−2 fold <E13.5-d4c7<2-fold) (FIG. 4F). The former contains genes such as Ddx4, Daz1, Brdt, Asz1, Dmrt1, Stra8, Sycp3, Syce1 and Smc1b, and is enriched with “germline gene”, and the latter contains Piwi12, Rp1101, Rp136 and Rhox genes (FIG. 4F). On the other hand, of the 252 genes commonly down-regulated in male/female germ cells in E13.5 and d4c7 PGCLC, 68 genes were only partially down-regulated compared to d6 PGCLC (E13. 5-d4c7<−2 fold), and 180 genes were completely downregulated in d4c7 PGCLC (−2 fold <E13.5-d4c7<2 fold). These findings demonstrate that, during expansion, PGCLC gradually acquires some of the germ cell maturation programs that appear in the embryonic gonad prior to sex differentiation, while essentially maintaining the properties of migrating PGC.

### Epigenetic Property of PGCLC During Expansion Culture

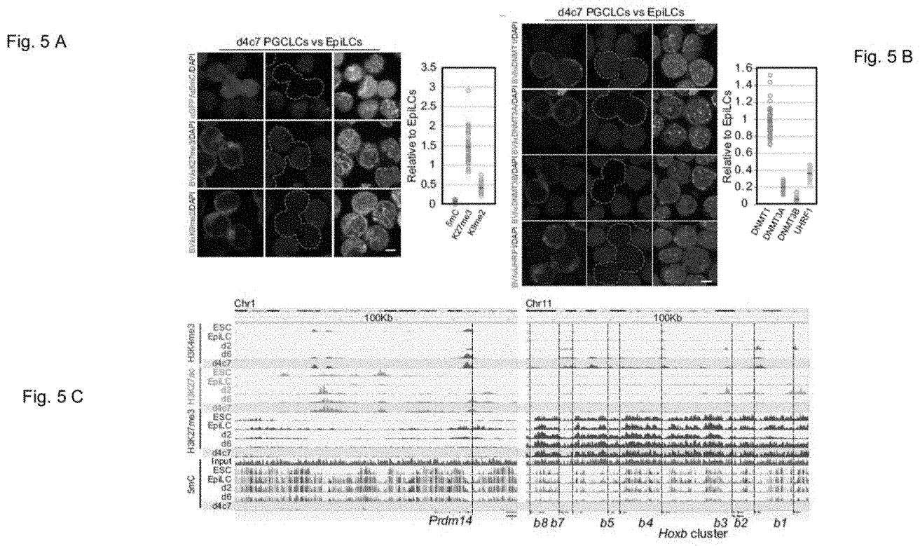

To explore the mechanisms governing the properties of cultured PGCLC, their epigenetic profile was determined. IF analysis revealed that d4c7 PGCLC had lower 5-methylcitron (5mC) levels compared to EpiLC (FIG. 5A). Consistent with the results of the transcriptome analysis, d4c7 PGCLC expressed DNMT1 at a similar level but the levels of DNMT3A/3B and UHRF1 were much lower compared to EpiLC (FIG. 5B). Furthermore, d4c7 PGCLC showed high and low histone H3 lysine 27 trimethylation [represents suppression by H3K27me3: Polycomb complex 2 (PRC2)] level and H3K9 dimethylation [H3K9me2: represents suppression by G9A/GLP] level, respectively, compared to EpiLC (FIG. 5A). Therefore, the epigenetic properties of d4c7 PGCLC seemed to be markedly similar to those of d6 PGCLC (Hayashi et al, 2011; Kurimoto et al, 2015), except that d4c7 PGCLC appears to have a much lower 5mC level than d6PGCLC.

Therefore, whole genome bisulfite sequencing (WGBS) was used to quantify the genome-wide level and distribution of DNA methylation in d4c3 and d4c7 PGCLC (derived from BVSC R8 ESC). In addition, chromatin immunoprecipitation sequence (ChIP-seq) followed by massively parallel sequencing was used to quantify genome-wide levels and distribution of H3K4me3 (indicating promoter activity), H3K27 acetylation (ac) (indicating activity enhancer), and H3K27me3 in d4c7 PGCLC (derived from BVSC R8 or BDF1-2ESC). In addition, the data was analyzed compared to the data on major cell types (ESCs, EpiLCs, and d2, d4 and d6 PGCLC) during the recently reported induction of PGCLC (Kurimoto et al., 2015; Shirane et al., 2016). Bisulfite sequencing does not distinguish 5mC and 5-hydroxymethylcytosine (5hmC) (Hayatsu&Shiragami, 1979), and since the 5hmC level during induction of PGCLC is almost negligible (Shirane et al, 2016), 5mC and 5hmC are collectively referred to as 5mC below. FIG. 5C shows WGBS and ChIP-seq track transitions around the Prdm14 locus and Hoxb cluster. Both active (H3K4me3 and H3K27ac) and inhibitory (H3K27me3) histone modifications showed relatively similar distributions between d6 and d4c7 PGCLCs (FIG. 5C). On the other hand, being strikingly consistent with IF analysis, 5mC was almost completely eliminated at both loci during the culture of PGCLC. This suggests that PGCLC expansion is a process that gradually eliminates 5 mC while maintaining histone modification.

### Comprehensive Elimination of DNA Methylation in Cultured PGCLC

Next, a more detailed analysis of the kinetics of 5mC level during PGCLC culture was performed. CpH (H=A, C, or T) methylation is restricted during the induction of PGCLC (Shirane et al, 2016) and has been shown to have no clear biological role in mammalian cells (Schubeler, 2015). Thus, 5mCs of CpG was focused on. As a key parameter for genome-wide DNA methylation status, the average of 5 mC levels of the whole unique sequence region was determined (unique region: 2 kb sliding window with 1 kb overlap). Promoters (high, medium, and low CpG density promoters: HCP, ICP, and LCP, respectively) (Weber et al., 2007), consensus sequence of repeat factor [long interspersed repeat 1 (LINE1); intracapsular A particle (IAP); Endogenous retroviral sequences (ERV) other than IAP; major and minor satellites], and the imprinted regulatory region (ICR) of the imprinted gene were determined separately. In addition, non-promoter CpG island (CGI), exons, intron, intergenic region, cell type-specific enhancer (Kurimoto et al., 2015), and 5mC level of “germline gene” CGI (Weber et al, 2007; Borgel et al, 2010; Kurimoto et al, 2015) were determined.

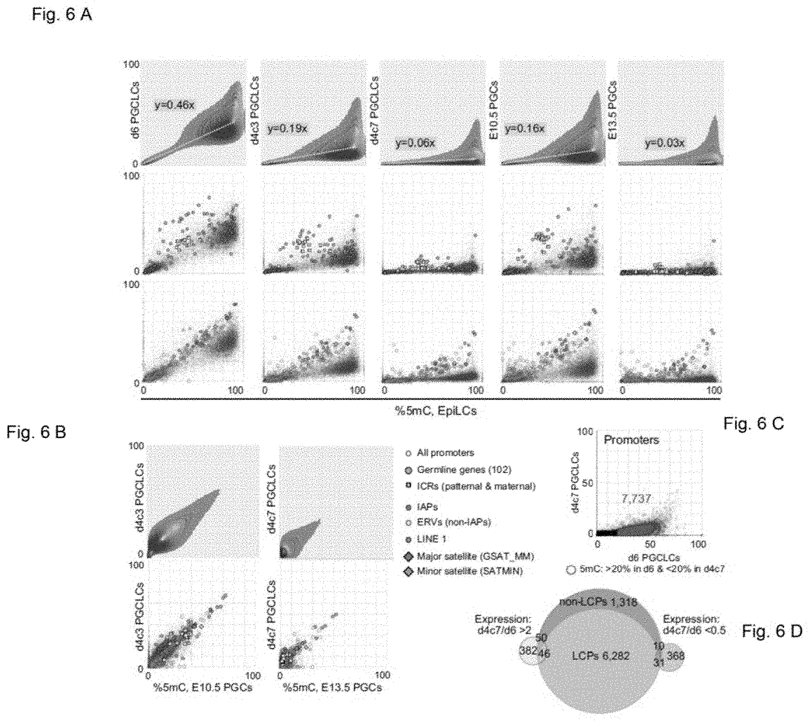

As previously reported, PGCLCs showed a serial dilution of 5mCs established with EpiLCs having DNA methylomes very similar to blastoderm, as a result of which d6 PGCLCs acquire about 37% on average of 5mC levels (state considered to be similar to the mobile PGC at about E9.0-9.5). Surprisingly, consistent with analysis of the Prdm14 and Hoxb loci, continuous dilutions of 5mC levels of d4/d6 cells were present in cultured PGCLC with different kinetics for unique region, repeat, and distinct regulatory elements. As a result, d4c7 cells had only about 6% mean 5mC level (FIG. 6A), comparable to E13.5 germ cells (Seisenberger et al, 2012; Kobayashi et al, 2013) with the lowest 5mC level throughout the germline cycle. Importantly, the 5mC distribution pattern in virtually all genomic elements, including repeat, promoter of “germline gene” resistant to demethylation, and ICR of imprinted gene, was markedly similar between d4c3 PGCLC and E10.5 PGC, and between d4c7 PGCLC and E13.5 germ cell. On the other hand, the 5mC distribution pattern between d4c3 PGCLC and ESC cultured using two kinase inhibitors (2i) showing similar 5mC level (Habibi et al, 2013; Shirane et al, 2016) was different. This indicates that demethylation of DNA in cultured PGCLC has a similar, if not the same, mechanism in PGCs in vivo, but is not similar to the mechanism induced in vitro by 2i. The “escapee” (5mC>20%) (Sepisenberger et al, 2012), which avoids DNA demethylation, was analyzed. It was found that the escapee showed a large overlap between d4c7 PGCLC and E13.5 germ cells, and the most part thereof exists near the IAP. Therefore, induction of PGCLC on the m220 feeder and culture thereof using FR10 comprehensively reconstituted DNA methylation reprogramming in PGC. Nevertheless, as demonstrated above, cultured PGCLC maintained the transcriptional state of essentially migratory PGC (FIG. 4B).

Therefore, the effect of promoter demethylation on transcriptional activation in d4c7 PGCLC was investigated. As many as 7,737 promoters were demethylated between d6 PGCLC and d4c7 PGCLC (5 mC>20% for d6, <20% for d4c7), reflecting global DNA demethylation in cultured PGCLC (FIG. 6C). Of the 478 genes upregulated in d4c7 PGCLC compared to d6 PGCLC, 96 had promoter demethylation, 27 were included in 104 partially upregulated genes (E13.5-d4c7>2-fold) (Ddx4, Daz1, Brdt, Asz1, Dmrt1, Stra8, Sycp3, Syce1, Smc1b and the like), and 34 were included in completely up-regulated 197 genes (−2-fold <E13.5-d4c7<2-fold) (Piwi12, Rp110 1, Rp136, Rhox gene and the like) (FIG. 6D). Among partially/completely up-regulated genes, the proportion of genes whose promoters were demethylated was higher than the proportion of genes whose promoters were demethylated among partially/completely down-regulated genes (FIG. 6D). Thus, it is concluded that demethylation of the promoter itself partially contributes to the activation of a limited number of specific genes in cultured PGCLCs.

### Compensatory Up-Regulation of H3K27 Me3 Demethylated Promoter in Cultured PGCLC

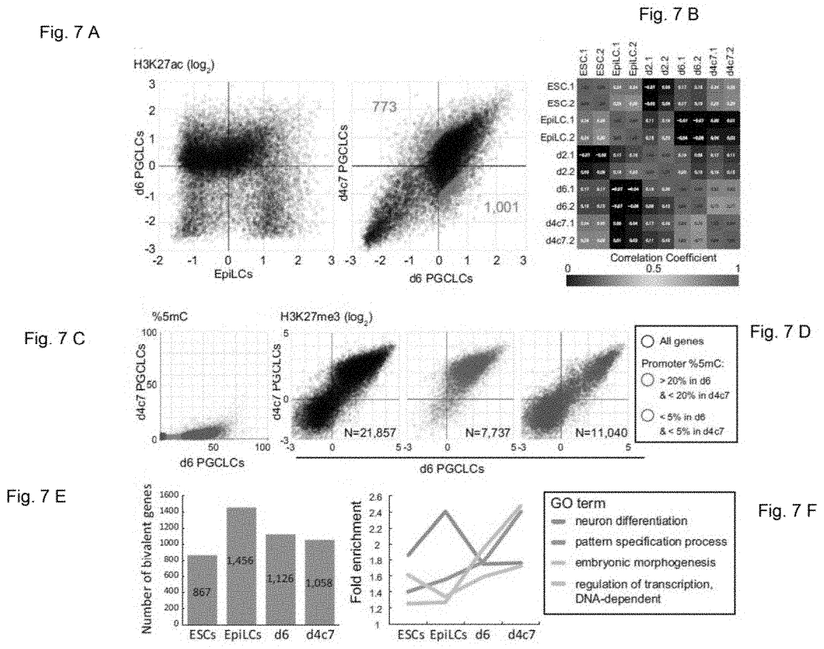

Next, the kinetics of the distribution of H3K4me3, H3K27ac and H3K27me3 during the induction and expansion of PGCLC was analyzed. Similar to the major cell types during PGCLC induction, high levels of H3K4me3 bound predominantly to HCP in d4c7 PGCLC, and H3K4me3 levels around the transcription start site (TSS) were positive for expression levels of related genes (Ohta et el., 2017. Fig. EV5A and B). This reflected the transcriptional similarity between d6 PGCLC and d4c7 PGCLC, and the H3K4me3 profile across the genome was similar between d6 PGCLC and d4c7 PGCLC. Importantly, the distribution of H3K27ac showing the use of enhancers and showing highly dynamic changes between different cell types (Calo & Wysocka, 2013) and during PGCLC induction (Kurimoto et al, 2015) was also similar between d6 PGCLC and d4c7 PGCLC (FIGS. 7A and B). This indicates that regulation of gene expression as well as gene expression itself is highly conserved during PGCLC culture. Nevertheless, the peak of H3K27ac specific for either d6 PGCLC or d4c7 PGCLC (15 kb from TSS and gene body) which would show potential regulatory differences between d6 PGCLC and d4c7 PGCLC was identified.

The distribution pattern of H3K27me3 also appeared to be very similar between d6 PGCLC and d4c7 PGCLC (FIGS. 7C and D). However, importantly, promoters with substantial demethylation between d6 PGCLC and d4c7 PGCLC (5mC>20% for d6, <20% for d4c7, 7737 promoter) showed a general tendency of having a higher concentration level of H3K27me3 than d6 PGCLC in d4c7 PGCLC, but promoters with unchanged 5mC levels did not show such tendency (FIGS. 7C and D). Despite showing substantial demethylation between EpiLC and d6 PGCLC, the aforementioned promoter did not show comprehensive changes in H3K27me3 enrichment levels between EpiLC and d6PGCLC. These findings indicate that extensive and almost complete promoter demethylation in cultured PGCs is at least partially compensated by the concomitant upregulation of H3K27me3 concentration levels, which may contribute to the maintenance of the transcriptional state of migratory PGC in cultured PGCLC.

A bivalent promoter that activates H3K4me3 and suppresses H3K27me3, which serves to a state that supports and triggers the activation of proper developmental (Voigt et al, 2013), was evaluated (for the definition of bivalent, see Materials and methods). Consistent with a previous report (Kurimoto et al, 2015), the number of bivalent genes was highest in EpiLC among j major cell types, and d6 and d4c7 PGCLC showed similar numbers of bivalent genes (FIG. 7E). However, the bivalent gene duplication between d6 PGCLC and d4c7 PGCLC was relatively moderate (˜519/1,058 (˜49%)). This is due, in part, to the technical difficulties inherent in accurately comparing the level of combination of two histone modifications in two different samples. Nevertheless, the divalent gene of d4c7 PGCLC showed a higher degree of enrichment in GO terms such as “pattern specification process” and “embryonic morphogenesis” as compared to that of d6 PGCLC (FIG. 7F). For example, d4c7 PGCLC acquired elevated H3K4me3 levels around the Hoxc cluster, even though the cluster was suppressed by high levels of H3K27me3. d4c7 PGCLC shows highly epigenetic “blank state” to genome-wide 5mC and H3K9me2 levels (FIGS. 5A and 6) (Kurimoto et al, 2015) at very low levels of developmental regulators. It is concluded that it has a highly balanced epigenome.

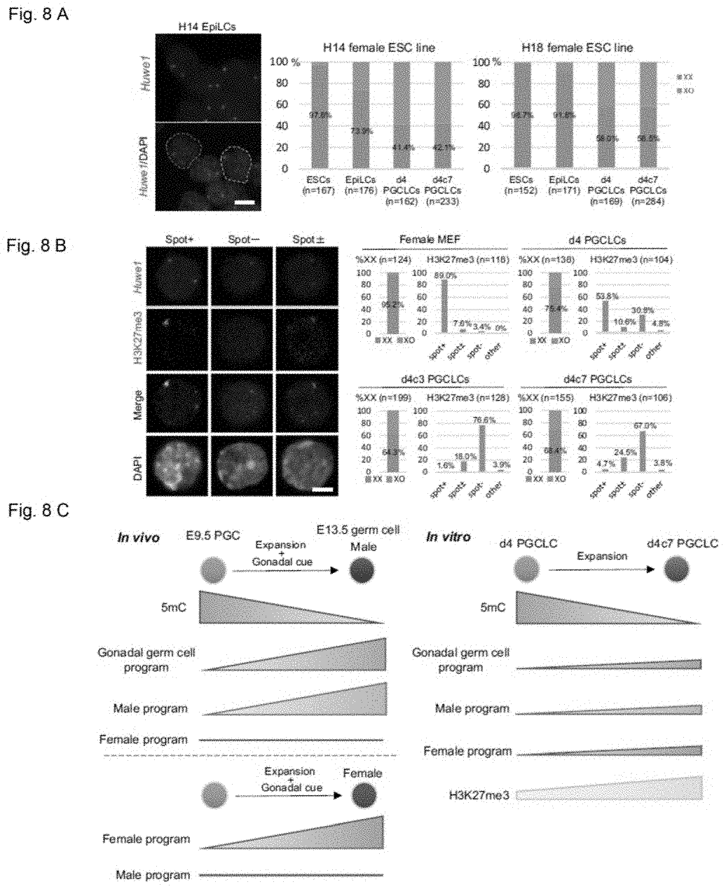

X-chromosome dynamics in female PGCLC and female ESC have two activated X chromosomes (XaXa) and show lower genome-wide 5mC levels compared to male ESCs (Zvetkova et al, 2005; Shirane et al, 2016). The majority of female EpiLCs carry XaXa, which undergoes X inactivation during floating aggregate formation, and female mPGCLC show one Xa and one inactive X chromosome (XaXi) (Hayashi et al, 2012; Shirane et al, 2016). Next, X chromosome dynamics during the growth of female PGCLC in culture were evaluated. Since female ESCs can lose one X chromosome to become XO (Robertson et al, 1983; Zvetkova et al, 2005), the extent of maintenance of the two X chromosomes during PGCLC induction and proliferation from female ESCs of two lines (BVSC H14 and H18 (129sv/C57BL/6 background)) were first investigated by the DNA fluorescence in situ hybridization (FISH) analysis of Huwe1 of an X-linked gene. As shown in FIG. 8A, two Xs were relatively well maintained during the differentiation from ESC to EpiLC, but one X tended to be lost during PGCLC induction (˜60% and ˜40% for XO: H14 d4 PGCLC and H18 d4 PGCLC, respectively). The XX/XO ratios were well conserved during PGCLC growth in culture (˜60% and ˜40% for XO: H14 d4c7 PGCLC and H18 d4c7 PGCLC, respectively).

Next, the present inventors investigated the Xa/Xi state during the expansion of PGCLC by evaluating H3K27me3 positivity on the X chromosome. As shown in FIG. 8B, about 95% of female mouse fetal feeders (MEFs) have two Xs and about 90% of the cells show a single H3K27me3 spot on one X, which is XaXi. The present inventors found that about 50% of XX d4 PGCLCs showed the XaXi state, while about 30% of XX d4 PGCLCs did not show the H3K27me3 spot on the X chromosome (FIG. 8B). Surprisingly, only a minority (<5%) of XX d4c3 and d4c7 PGCL showed XaXi status, about 70% of which did not show H3K27me3 spots on the X chromosome (FIG. 8B). This indicates that one Xi is reactivated during the expansion culture of PGCLC or in the process of reactivation, and in good agreement with the view that d4c7 PGCLC acquires an epigenetic “blank state”.

[II] Induction of Oocyte-Like Cell from PGC/PGCLC

For the detail information of the document cited below, see Miyauchi, H. et al., EMBO J., 36(21): 3100-3119 (2017).

### <Materials and Methods>

### Animal

All animal experiments were performed under the ethical guidelines of Kyoto University. BVSC (Acc. No. BV, CDB0460T; SC CDB0465T: http://www.cdb.riken.jp/arg/TG %20 mutant %20 mice %20 list.html), Stella-EGFP(SG) and mVH-RFP(VR) transgenic mice were established as previously reported (Payer et al, 2006; Seki et al, 2007; Ohinata et al, 2008; Imamura et al, 2010), and maintained primarily in the C57BL/6 background. Daz1-tdTomato(DT) mice were generated by injecting BVSCDT ESC(XY) into blastocysts (ICR) and then transferring same to a foster parent. ICR mice were purchased from SLC (Shizuoka, Japan). The noon of the day when the vaginal plug was identified was designated as 0.5 days of embryonic period (E).

To administer LDN-193189 to pregnant females (ICR), LDN-193189 (sm10559; Sigma-Aldrich) is dissolved in sterile water, and 2.5 mg LDN-193189 was injected intraperitoneally every 12 hours from E11 to E14 per kg body weight.

### ESC Culture/Induction and PGCLC Induction/Culture

H8 BVSC ESC(XX) (Hayashi et al, 2012), R8 BVSC ESC(XY) (Hayashi et al, 2011), L9 BVSCVR ESC(XX), L5 BVSCVR ESC(XY), BVSCDT ESC (XY; R8 subline), BDF1-2-1 BVSC ESC(XY) (Ohta et al, 2017), and Stra8knockout BVSC ESC (SK1,2,3; XY; BDF1-2-1 subline) were used for this study. L5 and L9 BVSCVR ESCs were established from blastocysts obtained by mating VR females (Imamura et al, 2010) with BVSC males (Ohinata et al, 2008) according to previously described procedures and adapted to feeder-free condition (Hayashi et al, 2011). The establishment procedures of BVSCDT and Stra8knockout ESC are described in the sections “Establishing the BVSCDT ESC” and “Establishing the Stra8 knockout ESC” below, respectively.

ESC culture and PGCLC induction was performed as described previously (Hayashi et al, 2011; Hayashi & Saitou, 2013) with some modifications. ESCs were maintained under 2i+LIF conditions on dishes coated with poly-L-ornithine (0.01%; Sigma) and laminin (300 ng/ml; BD Biosciences) or on mouse embryonic fibroblasts (MEFs). EpiLC was induced by plating 1.0×10⁵ ESC in N2B27 medium containing activin A (20 ng/ml; PeproTech), bFGF (12 ng/ml; Life Technology) and KSR (1%; Thermo Fisher), on the wells of a 12-well plate coated with human plasma fibronectin (16.7 μg/ml; Millipore). 42-46 hours after EpiLC induction, PGCLC was induced under suspension conditions by plating 2.0×10³ EpiLC in 200 μl of GK15 medium containing BMP4 (500 ng/ml; R & D Systems), LIF (1,000U/ml; Merck Millipore), SCF (100 ng/ml; R & D Systems) and EGF (50 ng/ml; R & D Systems) in wells of low-cell binding 96-well lipidure-coated plates (Thermo Fisher). The GK15 medium was constituted of GMEM (Thermo Fisher) containing 15% KSR, 0.1 mM NEAA, 1 mM sodium pyruvate, 0.1 mM 2-mercaptoethanol, 100 U/ml penicillin, 0.1 mg/ml streptomycin and 2 mM L-glutamine. PGC/PGCLC culture was performed as described previously (Ohta et al, 2017). In short, d4 PGCLC aggregates were collected, washed with PBS and dissociated with TrypLE Express (Thermo Fisher). They were then washed with DMEM/F12+0.1% BSA (Gibco) and filtered on a cell strainer (BD Bioscience) to remove large cell clumps. The samples were then centrifuged, resuspended in 0.1% BSA-PBS and sorted by FACS (Aria III; BD Bioscience). BV (+) cells were seeded on mitomycin C (MMC)-treated m220 feeder cells in PGC/PGCLC expansion medium.

The PGC/PGCLC expansion medium was constituted of 10% KSR, 2.5% FBS, 0.1 mM NEAA, 1 mM sodium pyruvate, 2 mM L-glutamine, 0.1 mM 2-mercaptoethanol, 100 U/ml penicillin, 0.1 mg/ml GMEM streptomycin, 10 pH forskolin, and 10 μM rolipram. The entire medium was changed every 2 days from c3. Cytokines/compounds for induction of female fate were provided from c3 to the end of culture. RA and BMP concentrations used were 100 nM and 300 ng/ml, respectively, unless otherwise specified. Brightfield and fluorescence images were captured using an IX73 inverted microscope (Olympus).

### Establishment of BVSCDT ESC

To construct a donor vector for the production of Daz1-tdTomato (DT) knockin ESCs, the homology arms of Daz1 (fragments from 1,048 bp upstream and 1,247 bp downstream of the stop codon, respectively) were amplified from genomic DNA of R8 BVSC ESC by PCR (Primers) and subcloned into pCR2.1 vector (TOPO TA Cloning; Life Technologies). The P2A-tdTomato fragment with the Pgk-Puro cassette flanked by LoxP sites was amplified by PCR from a previously reported vector (Sasaki et al, 2015) and inserted in frame into 3′-terminal (containing homology arm) of the Daz1 coding sequence of the subcloned vector by using GeneArt Seamless Cloning & Assembly Kit (Life Technologies). The stop codon was removed for expression of the in-frame fusion protein.

TALEN constructs targeting sequences flanking the stop codon of Daz1 were generated using the GoldenGate TALEN and TAL Effector kit (Addgene #1000000016) as previously described (Sakuma et al, 2013; Sasaki et al, 2015). The TALEN activity was assessed by single-strand annealing (SSA) assay.

Donor vector (5 μg) and TALEN plasmid (10 μg each) were introduced into R8 BVSC ESC by electroporation using NEPA21 type II electroporator (Nepagene). Single colonies were picked after puromycin selection and random or targeted integration was assessed by PCR (Primers), followed by Southern blot analysis. A strain with correct targeting was transfected with a plasmid expressing Cre recombinase to remove the Pgk-Puro cassette.

### Establishment of Stra8 Knockout Esc

A vector expressing the Cas9 nickase (Addgene #42335) fused in-frame with the reporter gene GSG-p2A-mCherry was constructed (the mCherry used contained a silent mutation, 432G>A). According to the reported protocol (Ran et al, 2013a, b), two pairs of oligonucleotides (Primers) for targeting exon 6 of Stra8 were annealed, phosphorylated and ligated separately to the above vector digested with Bbs1 (NEB). Nickase activity was assessed by SSA assay. A pair of nickase plasmids (200 ng each) was introduced into BDF1-2-1 BVSC ESC by electroporation using NEPA21 type II electroporator. ESCs were dissociated 2 days after transfection, single cells expressing high levels of mCherry and also expected to express high levels of Cas9 nickase were sorted by FACS and seeded on MEFs in single wells of 96-well plates such that each well contained a single clone. Clones were cultured, expanded, and disruption of the Stra8 locus in the clones was assessed by Sanger sequencing (Primers) of PCR products of the relevant region. Stra8 knockout was confirmed by Western blot and IF analysis.

### Ex Vivo Culture of PGC or Fetal Gonadal

For PGC culture, fetal gonads of SG mice at E11.5 (without gender discrimination) were cut and dissociated. SG (+) PGCs were selected by FACS, plated on m220 feeder cells, and cultured in PGC/PGCLC expansion medium. Reagents for induction of female fate were provided from c0. For fetal gonadal culture, the ovary of the embryonic stage, including the E11.5 [identified by PCR (Primers)] mesonephros, was removed and cultured under conditions of the gas-liquid interface on the culture medium insert (353095; BD Falcon). The medium used was DMEM containing 10% FBS, 100 U/ml penicillin, 0.1 mg/ml streptomycin and 2 mM L-glutamine. Small molecule inhibitors were provided with medium from c0.

### Fluorescence Activation Cell Sorting, Cell Cycle Analysis, and Cell Count

The preparation of d4/c0 PGCLC for FACS is described in “ESC culture/induction and PGCLC induction/culture”. To isolate germ cells in vivo, fetal gonads of BVSC, VR or SG mice were cut and processed for FACS according to the procedure described for d4/c0 PGCLC. Culture PGCLC was also prepared by the same method except that, after dissociation, the cells were washed with 0.1% BSA-DMEM containing 100 μg/ml DNase (Sigma-Aldrich), lysed DNA from dead cells was digested to prevent the formation of cell/debris clumps. Fluorescent activity of BV/SG, SC, or DT/VR was detected with FITC, Horizon V500 or PE-Texas Red channels, respectively. FACS data was analyzed using FlowJo or FACS Diva software package.

Cell cycle analysis was performed using the Click-iT EdU Flow Cytometry Assay Kit (C10424; Thermo Fischer Scientific) according to the manufacturer's instructions. Cultured PGCLC was treated with 10 μg/ml EdU for 30 min to 2 hr and analyzed by FACS.

Cultured PGCLC was stained with chicken anti-GFP antibody, followed by Alexa Fluor 633-goat anti-chicken antibody and analyzed using Cellavista instrument (SynenTec) (Ohta et al, 2017).

### Cytokine/Compound

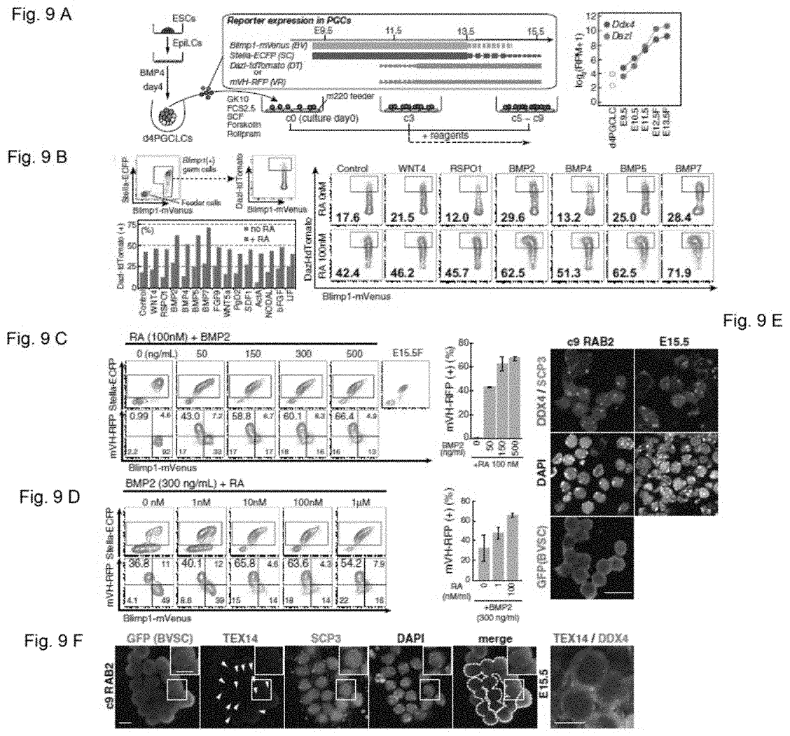

The cytokines/compounds used to screen for activities involved in female fate induction were as follows (FIG. 9): 100 nM all trans retinoic acid, 500 ng/ml WNT4 (R&D Systems), 500 ng/ml RSPO1 (R&D Systems), 100 ng/ml FGF9 (R&D Systems), 500 ng/ml PgD2 (Cayman), 25 ng/ml activin A, 100 ng/ml NODAL (R&D Systems), 500 ng/ml SDF1 (R&D Systems), 50 ng/ml bFGF, 500 ng/ml BMP2 (R&D Systems), 500 ng/ml BMP4 (R&D Systems), 500 ng/ml BMP5 (R&D Systems), 500 ng/ml BMP7 (R&D Systems), 250 ng/ml WNT5a (R&D Systems) and 1,000 U/ml LIF. In the signal inhibitory experiment, LDN193189 (#04-0074; Stemgent) and BMS493 (B6688; Sigma-Aldrich) dissolved in DMSO were used as ALK2/3 inhibitor and RAR inhibitor, respectively.

### Immununofluorescence (IF) Analysis

Immunofluorescence (IF) analysis was performed as described previously (Hayashi et al, 2012). The following primary antibodies were used: chicken anti-GFP (ab13970; Abeam), rabbit anti-DDX4 (ab13840; Abeam), mouse anti-DDX4 (ab27591; Abeam), rabbit anti-DAZL (ab34129; Abeam), goat anti-DAZL (sc-27333; Santa Cruz), rabbit anti-STRA8 (ab49602; Abeam), mouse anti-SYCP3 (ab97672; Abeam) and rabbit anti-TEX14 (ab41733; Abeam)IgG. The following secondary antibodies were used: Alexa Fluor 488-goat anti-mouse or chicken IgG; Alexa Fluor 568-goat anti-rabbit IgG and Alexa Fluor 633-goat anti-mouse, anti-chicken IgG; Alexa Fluor 488-donkey anti-mouse IgG; Alexa Fluor 568-donkey anti-rabbit IgG and Alexa Fluor 633-donkey anti-goat IgG. IF images were acquired using a confocal microscope [FV1000 (Olympus) or LSM780 (Zeiss)].

### Spread Analysis of Meiotic Chromosome in Fetal Oocyte-Like Cell

Spread preparation and IF analysis were performed with slight alteration and as described previously (Yamashiro et al, 2016). c9 RAB2 cells were sorted by FACS, sorted cells were washed with PBS and treated with hypotonic extraction solution at 25° C. for 1 hr. The primary antibodies used are as follows: goat anti-SCP3 (1:250; sc-20845; Santa Cruz), mouse anti-γH2AX (1:1,000; 05-636; Millipore) and rabbit anti-SCP1 antibody (1:250; NB300-229; Novus). The secondary antibodies used are as follows: Alexa Fluor 488-donkey anti-goat IgG (A11055; Thermo Fisher), Alexa Fluor 568-donkey anti-rabbit IgG (A10042; Thermo Fisher), and Alexa 647-donkey anti-mouse IgG (A31571; Thermo Fisher). The present inventors counted SYCP3 (+) cells. The definition of the meiotic stage was as follows: at least 80% of the chromosomes, thread stage: γH2AX (+) and SYCP1 (−); mating stage: γH2AX (+) and SYCP1 (+). Thick thread period: SYCP1 (++).

### Southern Blot Analysis

Southern blot analysis was performed as described previously (Nakaki et al, 2013). Briefly, 10 μg of genomic DNA was digested with restriction enzymes, the resulting DNA fragment was electrophoresed on a 0.9% agarose gel, transferred to Hybond N+membrane (RPN303B; GE Healthcare) and baked for crosslinking. A DIG-labeled probe for tdTomato and 5 and 3 prime sides of the relevant region of Daz1 were generated by PCR (PCR DIG Labeling Mix; Sigma-Aldrich) (Primers). Images were captured using LAS4000 (Fujifilm).

### Western Blot Analysis

For Western blot analysis, 5×10⁴ cells were lysed in SDS sample buffer [62.5 mM Tris-HCl (pH 6.8), 2% SDS, 10% glycerol, 0.025% bromophenol blue and 0.14 M-mercaptoethanol] at 90° C. for 5 min. For detection of phosphorylated (p)SMAD1/5/8, the cells were treated with PhosSTOP (Roche) and complete protease inhibitor cocktail (Roche) before lysis. The extracted protein was separated by SuperSep Ace 10-20% gel (Wako), blotted on iBlot2 PVDF transfer membrane (Thermo Fisher) by iBlot2 dry blotting system (Thermo Fisher), and incubated with the primary antibody: rabbit anti-STRA8 IgG (ab49405; Abeam), mouse anti-α tubulin (T9026; Sigma-Aldrich), rabbit anti-pSMAD1/5/8 IgG (#9511; CST), or rabbit anti-SMAD1 IgG (#97435 CST). Primary antibodies were detected with goat anti-rabbit IgG or sheep anti-mouse IgG conjugated to HRP (A0545, M8642; Sigma-Aldrich), followed by Chemi-Lumi One Super (Nacalai). Chemiluminescence image acquisition and analysis was performed using the Fusion Solo system and Fusion-Capt software (M & S Instruments).

### Transcriptome Analysis

E14.5 and E15.5 male and female germ cells were used as VR (+) cells, cultured PGCLC were used as SC(+) cells, and collected by FACS, and total RNA of the sample was analyzed using RNeasy Micro Kit (74004; QIAGEN) according to the manufacturer's instructions. cDNA synthesis, library construction, and analysis with Nextseq 500 (Illumina) from 1 ng of total RNA from each sample were performed according to the methods described previously (Nakamura et al, 2015; Ishikura et al, 2016). The germline cDNAs from E9.5 to E13.5 prepared in previous studies (Kagiwada et al, 2013; Ohta et al, 2017) were also analyzed by the Nextseq 500 sequencer system. All readings were converted to expression levels as previously described (Nakamura et al, 2015). Briefly, all reads were treated with cutadapt-1.3 (Martin, 2011) to remove VI and V3 adapter sequences and poly A sequences. The resulting reads of 30 bp or above were mapped to the mm10 genome using TopHat1.4.1/Bowtie1.0.1 using the “-no-coverage-search” option (Kim et al, 2013). The mapped reads were then converted to expression levels (RPM) using cufflinks-2.2.0 with “-compatible-hits-norm”, “no-length-correction”, “-max-mle-iterations 50000”, and “library”-type fr-secondstrand option and mm1-reference gene annotation of maximum 10-kb at 30 endo. The total set of genes analyzed was the same as in the previous study (Ishikura et al, 2016). Transcriptome analysis was performed using the R software package version 3.2.1 with the gplots package and Microsoft Excel. First, the raw expression data was converted to log₂ (RPM+1) values and genes with expression values >2 in at least one sample were defined as expression unless particularly indicated. Data processing (e.g., DEG identification) was performed by using the average of the biological replicates, with the exception of heat maps, clustering and PGA construction. The gplots package (heatmap.2) was used to create the heatmap. Gene Ontology (GO) analysis was performed using the DAVID 6.7 website (https://david.ncifcrf.gov) (Huang da et al, 2009).

### Quantitatively (g) PCR

qPCR was performed using CFX384 (Bio-Rad) and Power SYBR Green (ABI, Foster City, Calif.) according to the manufacturer's instructions. Template cDNA was prepared as described in the “Transcriptome analysis” section and the primers used are listed in Primers.

### Analysis of DNA Methylation of Promoter

Whole genome bisulfite sequencing (WGBS) data for ESCs, EpiLCs, and PGCLCs were obtained from a previous study of the present inventors (Shirane et al, 2016; Ohta et al, 2017) and those of KIT (−) and KIT (+) spermatogonia were obtained from a previous study (Kubo et al, 2015). Here, the present inventors defined the promoter as a region from 900 bp upstream to 400 bp downstream of the transcription start point (TSS), and calculated all percent 5 methylcytosine values (% 5 mC) from the average value of CpG. The read depth was 4-200, as previously described (Ishikura et al, 2016). In the analysis in this study, a promoter with at least one CpG site was used.

### Primer

The primer sequences used in this study were as follows: Sex-typing:

Ubal forward:
(SEQ ID NO: 1)
TGGATGGTGTGGCCAATGCYCT
reverse:
(SEQ ID NO: 2)
CCACCTGCACGTTGCCCTTKGTGCCCAG
Dazl-tdTomato vector construction:
Dazl forward:
(SEQ ID NO: 3)
AGAATCTGGTCCACAGGAAAAGGGCCTGAT
reverse:
(SEQ ID NO: 4)
AGGCTTAGCCCCTGAGCTGCTTGTTAACTG
genotype forward:
(SEQ ID NO: 5)
AATAGACCCTAACCAGTTGGTGCAT
reverse:
(SEQ ID NO: 6)
AAGAGTAAGTGGAATCCATGTTGAAGGA
random incorporation check forward:
(SEQ ID NO: 7)
CCGGATGAATGTCAGCTACTGGGCTATCTG
reverse:
(SEQ ID NO: 8)
TTCGGGGCGAAAACTCTCAAGGATCTTACC
For Stra8 knockout cell generations:
nickasel forward:
(SEQ ID NO: 9)
CACCgGAGATGGCGGCAGAGACAAT
reverse:
(SEQ ID NO: 10)
AAACATTGTCTCTGCCGCCATCTCc
knickers2 forward:
(SEQ ID NO: 11)
CACCgCCTGTGGCAGACTCTCTCTG
reverse:
(SEQ ID NO: 12)
AAACCAGAGAGAGTGCCACAGGc
sequence check forward:
(SEQ ID NO: 13)
TAAGGCCAGGGGAAGGCAGAC
reverse:
(SEQ ID NO: 14)
CGAAGGGCCATCTCACAGGGTC
Southern blot analysis of Dazl-tdTomato gene locus:
5′ probe forward:
(SEQ ID NO: 15)
GGAGGCAAAGACAGGATCCTAAGCAAACTA
reverse:
(SEQ ID NO: 16)
TCCCTAGTGCTCTATAAAATTGAGGTAATT
tdTomato probe forward:
(SEQ ID NO: 17)
CCGCCGACATCCCCGATTAC
reverse:
(SEQ ID NO: 18)
GCAGTTGCACGGGCTTCTTG
3′ probe forward:
(SEQ ID NO: 19)
ACCAGAAAACTAAGAAGTGTCCTGAA
reverse:
(SEQ ID NO: 20)
AGACAGGACAGTAAGTCAGTGATGCT
qPCR analysis:
Id1 forward:
(SEQ ID NO: 21)
CAACAGAGCCTCACCCTCTC
reverse:
(SEQ ID NO: 22)
AGAAATCCGAGAAGCACGAA
Id2 forward:
(SEQ ID NO: 23)
CACAAAGGTGGAGCGTGAATAC
reverse:
(SEQ ID NO: 24)
GCATTCAGTAGGCTCGTGTCAA
Ddx4 forward:
(SEQ ID NO: 25)
CAGCTTCAGTAGCAGCACAAG
reverse:
(SEQ ID NO: 26)
CATGACTCGTCATCAACTGGA
Daz1 forward:
(SEQ ID NO: 27)
GATGGACATGAGATCATTGGAC
reverse:
(SEQ ID NO: 28)
ATACCAGGGAGCAATCCTGAC
Stra8 forward:
(SEQ ID NO: 29)
GCCGGAGAAGGAGGAGATTAAA
reverse:
(SEQ ID NO: 30)
AGCAGCCTTTCTCAATGAGTCT
Sycp3 forward:
(SEQ ID NO: 31)
GTGTTGCAGCAGTGGGAAC
reverse:
(SEQ ID NO: 32)
GCTTTCATTCTCTGGCTCTGA
Prdm9 forward:
(SEQ ID NO: 33)
CCTGGCTACAAATTCTCATTTTC
reverse:
(SEQ ID NO: 34)
TGTTTTTGTTTGTTTTGTTTTGGGA
Spo11 forward:
(SEQ ID NO: 35)
AGCATGAAGTGTCTCACTAGCA
reverse:
(SEQ ID NO: 36)
CATTAACAGGGCAAGGCACCTA
Nanos2 forward:
(SEQ ID NO: 37)
AGAGAAGAATGCCAGTTGGGTT
reverse:
(SEQ ID NO: 38)
ACAACGCTTTATTCAGCAGCAG
Dnmt31 forward:
(SEQ ID NO: 39)
TCGGGTTTCTCTCCTGTTTG
reverse:
(SEQ ID NO: 40)
GTTATCCCACCGGGAACTTG
Ppia forward:
(SEQ ID NO: 41)
TTACCCATCAAACCATTCCTTCT
reverse:
(SEQ ID NO: 42)
AACCCAAAGAACTTCAGTGAGAGC
Rp1p0 forward:
(SEQ ID NO: 43)
CAAAGCTGAAGCAAAGGAAGAG
reverse:
(SEQ ID NO: 44)
AATTAAGCAGGCTGACTTGGTTG
Pou5f1 forward:
(SEQ ID NO: 45)
GATGCTGTGAGCCAAGGCAAG
reverse:
(SEQ ID NO: 46)
GGCTCCTGATCAACAGCATCAC

### Accession NO.

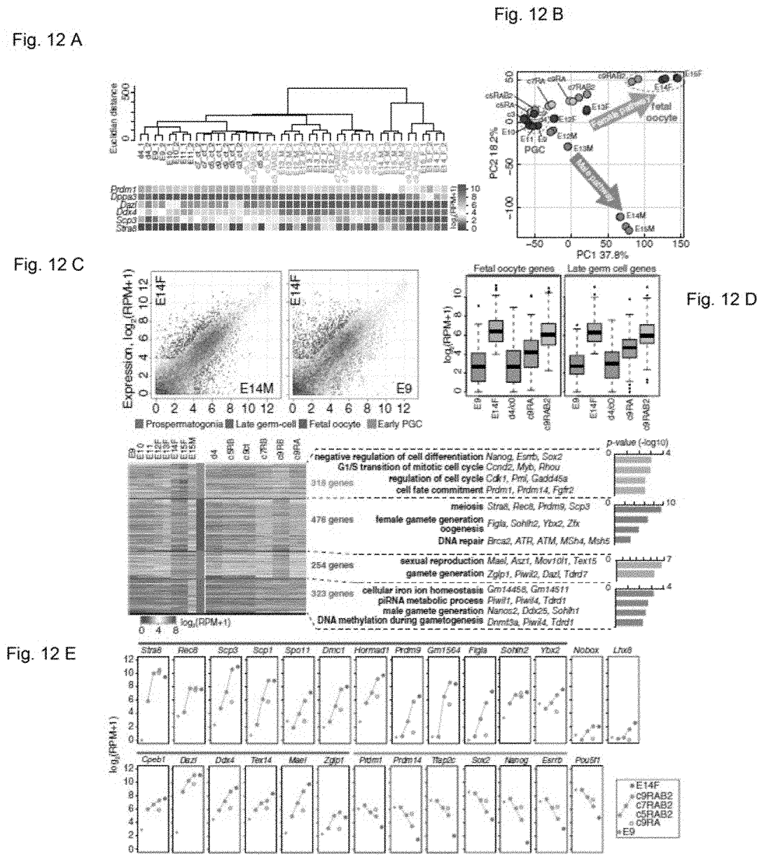

The accession numbers of the data used in this study are as follows: RNA-seq data of E10.5 and E11.5 PGC and E13.5 female germ cells in FIG. 12 (GEO:GSE74094) (Yamashiro et al, 2016), RNAseq data for E9.5 PGC and E12.5 female germ cells in FIG. 12 (GEO: GSE87644) (Ohta et al, 2017), RNA-seq data for d4 PGCLCs in FIG. 12 (GEO: GSE67259) (Sasaki et al, 2015), RNAseq data, microarray data for male and female support cells in E11.5, E12.5 and E13.5 (GEO: GSE27715) (Jameson et al, 2012), WGBS data for ESCs, EpiLCs and d4 PGCLCs (DDBJ: DRA003471) (Shirane et al, 2016), WGBS data for c7 PGCLCs (DDBJ: DRA005166) (Ohta et al, 2017) and WGBS data for P7 KIT⁻ SG and KIT⁺ SGs (DDBJ: DRA002477) (Kubo et al, 2015). Accession number for RNA-seq data of PGCs at E9.5, E10.5 and E11.5, female and female germ cells at E12.5, E13.5, E14.5 and E15.5, and PGCLCs cultured under the conditions described, is GSE94136 (GEO database).

### <Results>

### System to Analyze Sex Determination Mechanism of Germ Cells

As described in detail in [I] above, the present inventors developed a system to grow PGCLCs up to 50-fold during 7 days of culture on m220 feeder cells in the presence of stem cell factor (SCF) and cAMP signaling forskolin and loripram stimulating factor (FIG. 9A). Under these conditions, PGCLC gradually erases DNA methylome while maintaining the transcriptome of mobile/early gonadal PGC that is not committed to sex, and acquires a genomic overall DNA methylation level (about 5%)/pattern that is comparable to the germ cell pattern at E13.5 which is committed to either male or female fate (Spiller & Bowles, 2015). Thus, reprogramming of DNA methylation and sex differentiation in germ cell is genetically separable, and PGCLC cultured under these conditions may serve as a system to explore the mechanism of sex differentiation.

The present inventors investigated this possibility by focusing on differentiation into a female pathway characterized by entry into early meiosis. One prerequisite for PGCLC differentiation into the female pathway is the acquisition of late PGC property characterized by the expression of genes such as Daz1 and Ddx4 [also known as mouse vasa homolog (mVH)], both of which are expressed at low levels in PGCLC/migratory PGC and show progressive up-regulation in germ cells up to E13.5 (FIG. 9A right) (Fujiwara et al, 1994; Cooke et al, 1996; Ohta et al, 2017). In addition, Daz1 has been proposed to act as a “licensing” factor for germ cell sex differentiation (Lin et al, 2008; Gill et al, 2011). Thus, the present inventors generated ESC strains (BVSCDT ESC and BVSCVR ESC) under the control of Blimp1 (also known as Prdm1), Stella (also known as Dppa3), and Daz1 or Ddx4 (DdmVH) that express mVenus, ECFP, and tdTomato or RFP, respectively (Blimp1-mVenus may be abbreviated as BV, Stella-ECFP as SC, daz1-tdTomato as DT, and VH-RFP as VR, respectively) (Materials and methods). Blimp1 shows PGC fate determination (Ohinata et al, 2005), while Stella shows expression in established PGCs (Saitou et al, 2002), whereas BV and SC repeat the expression of Blimp1 and Stella, respectively (Ohinata et al, 2008). The present inventors also confirmed that DT and VR repeated the expression of Daz1 and Ddx4 from late PGC, respectively (Imamura et al, 2010). The present inventors attempted to establish the conditions that lead to the expression of DT or VR in cultured PGCLCs (followed by/combined with entry into female fate and down-regulation of BVSC expression). Since both XY germ cells and XX germ cells can take female fate (Evans et al, 1977; Taketo, 2015), they used both XY ESCs and XX ESCs as starting materials showing similar results (see below).

The present inventors first induced BVSCDT ESC(XY) into PGCLC and isolated BV-positive (+) day 4 (d4) PGCLCs by fluorescence-activated cell sorting (FACS) for growth culture. At day 3 (c3) of culture, when PGCLCs were growing exponentially, cultures were given a panel of cytokines that may affect sex determination in the presence or absence of RA, and at c7, their effects on BV or DT expression were assessed by FACS (forskolin, loripram, and SCF were given throughout the culture) (FIGS. 9A and B). Under control conditions (no addition of cytokine and RA), BV (+) c7 PGCLCs showed, on average, relatively low DT expression (FIG. 9B). Interestingly, the addition of RA increased DT levels in the BV (+) cell population (FIG. 9B). Particularly, the combination of RA and one of the BMPs (BMP2, 4, 5, and 7) strongly activated DT and simultaneously down-regulated BV, though the other cytokines were not tested (FIG. 9B). This suggests that RA and BMP induce PGCLC into a late germ cell phenotype.

Bmp2 is strongly expressed in response to the important feminizing factor Wnt4 in anterior granule membrane cells, which can guide the fate of female germ cells (Yao et al, 2004; Jameson et al, 2012). Therefore, the present inventors next investigated whether RA and BMP2 also elevated VR in cultured PGCLCs induced from BVSCVR ESC (XX). The present inventors cultured BV(+)d4 PGCLCs with varying concentrations of RA and BMP2 at c3 and examined their effects on BVSCVR expression at c9 (FIGS. 9C and D). RA alone did not significantly alter BVSC expression and did not activate VR (FIG. 9C). This suggests that Daz1 and Ddx4 expression is differentially regulated. Notably, the combined addition of RA and BMP2 induced BVSC downregulation and firm VR upregulation, and the rate of VR(+) cell induction increased in a BMP dose-dependent manner (FIG. 9C). Interestingly, BMP2 alone down-regulated BV and activated VR, and the extent of BVSC down-regulation and VR activation increased in a RA dose-dependent manner (FIG. 9D) (see below).

By immunofluorescence (IF) analysis, the present inventors analyzed the expression of DDX4 and SCP3 (also known as SYCP3) (Yuan et al., 2000), an important component of the synaptonem complex and a marker of early meiosis, at c9 in cultured PGCLC with RA and BMP2 [Yuan et al., 2000], which were induced from BVSC ESC (XX) to secure one fluorescent channel]. BV or SC-positive (sometimes abbreviated as “BV/SC(+)” in the present text) cells expressed DDX4 and SCP3 in a very similar fashion to E15.5 protoplasts: DDX4 localized specifically to the cytoplasm and SCP3 showed a distinct pattern of localization indicating synaptonem complex formation. Furthermore, DDX4/SCP3(+) cells appeared to be interconnected, reminiscent of the formation of oocyte cysts (FIG. 9E) (Pepling & Spradling, 1998). Indeed, cyst-like structures showed expression and localization of the cytoplasmic cross-linking marker TEX14 (Greenbaum et al., 2009; Lei & Spradling, 2016), specifically at cell-to-cell contact sites (FIG. 9F). When combined with RA, BMP4, 5 and 7 were also able to induce VR/DDX4 and SCP3(+) cells. Thus, the combined action of RA and BMP signaling may lead cultured PGCLCs to the fate of the female.

### BMP and RA Commit PGCLC to Female Fate

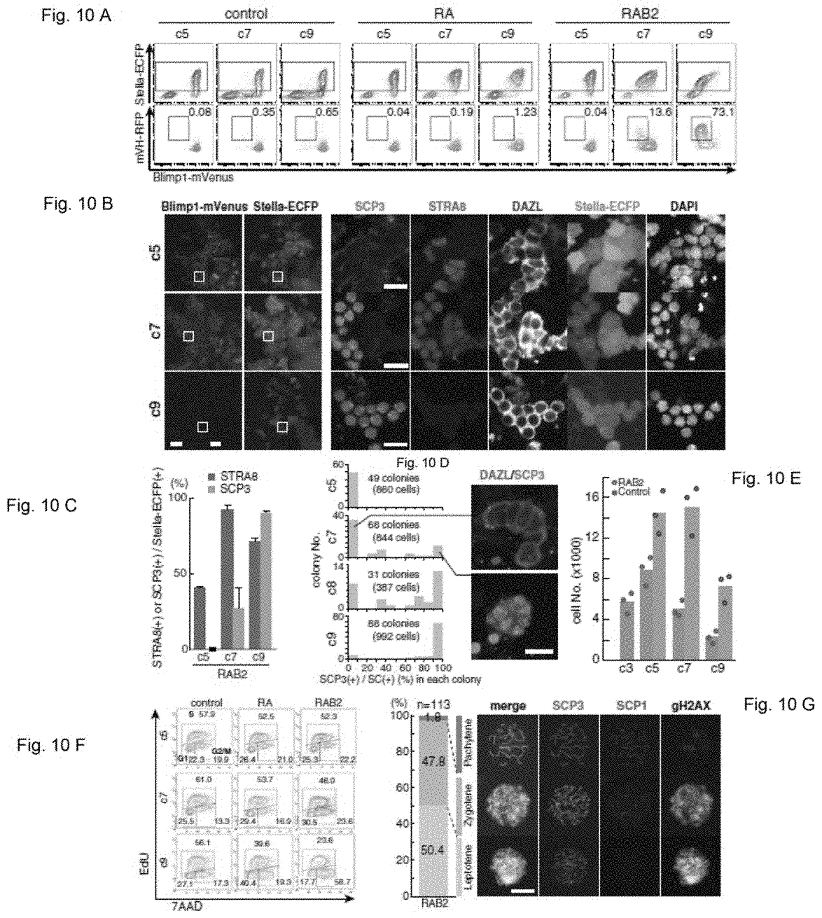

The present inventors further investigated the effects of RA and BMP signaling on PGCLC induced from BVSCVR ESC (XX), since VR shows a more specific response to RA and BMP (FIGS. 9C and D). Culture using RA and BMP2 after c3 resulted in down-regulation of BVSC at c7 and significantly reduced BVSC at c9, but not with RA alone (FIG. 10A). Conversely, under this condition, VR began to be activated by c7, and the majority (approximately 70%) of SC(+) cells showed VR at c9 (FIG. 10A). The present inventors confirmed that PGCLC stimulated by RA and BMP2 up-regulated phosphorylated (p)SMAD1/5/8 and up-regulated expression of Id1 and Id2, which are direct downstream targets of BMP signaling (Hollnagel et al, 1999; Korchynskyi & ten Dijke, 2002; Lopez-Rovira et al, 2002). Administration of LDN193189 (Cuny et al., 2008), a selective inhibitor of the ALK2/3 receptor, blocked such effects. This indicates that PGCLC is capable of activating the BMP signaling pathway (see also below).

IF analysis showed that PGCLCs cultured with RA and BMP2 initiated expression of the important mitogenic factor STRA8 (Baltus et al, 2006; Anderson et al, 2008; Dokshin et al, 2013; Soh et al, 2015) early in c5 [˜40.7%/SC (+) cells], while the majority (˜91.7%) of SC (+) cells expressed STRA8 in c7, some of which (˜27.1%) were SCP3 (+) (FIGS. 10B and C). Notably, in c9, more than 90% of SC (+) cells expressed SCP3 with synaptome-like structure formation, and STRA8 began to be attenuated (FIGS. 10B and C). Interestingly, SCP3 was up-regulated in a syncytial fashion in all SC(+) cells containing different colonies at a given time during culture (FIGS. 10B and D). From c5 to c9, SC (+) cells showed clear positivity for DAZL (FIG. 10B).

Entry of meiosis is characterized by pre-meiotic DNA replication and arrest in the quadruple chromosome (4C) state (Baltus et al., 2006). In the presence of RA and BMP2, BV/SC(+) cells showed an enhancement of proliferation up to c5, stopped proliferation by c7, and decreased to about half of the peak number thereof by c9 (FIG. 10E). Cell cycle analysis revealed that, at c7, a substantial portion (˜46.0%) of BV/SC(+) cells either replicated their DNA (about 23.6%) or was in the 4C state (˜23.6%) (FIG. 10F). In c9, surprisingly, the majority (about 58.7%) of BV/SC(+) cells were in the 4C state (FIG. 10F). This was not the case when PGCLCs were cultured under control conditions or with RA alone (FIG. 10F). Taken together, these findings indicate that PGCLC cultured with RA and BMP2 takes the fate of female and enter the early meiotic period. Indeed, diffusion analysis revealed that SC(+) cells in the pre-meiotic phase of meiosis progressed to the filamentous phase (filamentous phase: about 50.4%; junctional phase: about 47.8%; filamentous phase: about 1.8%) (FIG. 10G).

Under the conditions adopted by the present inventors, RA alone was insufficient to induce female germ cell fate. PGCLC cultured with RA activated DT/DAZL to some extent and up-regulated STRA8 [˜77.7% of BV/SC(+) cells expressed STRA8], but RA did not activate VR/DDX4 or SCP3 (FIGS. 9B and C, 10A), and RA-doped BV/SC(+) cells did not go into meiosis even in c9, but appeared to turn the cell cycle (FIG. 10F). Unexpectedly, BMP2 alone induced VR and SCP3 (+) mitotic cells in c9, although less effective than RA and BMP2 (FIG. 9D). Since the cultures of the present inventors contained 10% KSR and 2.5% fetal bovine serum (FCS) (FIG. 9A), the presence of BMP activity in such components may be negligible, while the presence of RA activity (More et al., 2016) may confer on PGCLCs the ability to take the fate of female in response to exogenous BMP signaling. Thus, culture of PGCLC with an inhibitor (BMS493) against BMP2 and RA receptors (RAR) (Koubova et al., 2006) abolished upregulation of VR/DDX4 as well as induction of SCP3. Similarly, inhibition of BMP signaling by LDN193189 blocked RA- and BMP2-induced VR activation and meiotic entry in a dose-dependent manner. The present inventors conclude that RA and BMP signal transduction is necessary and highly likely sufficient to induce PGCLC into the female pathway, and that RA or STRA8 alone is not capable of such induction.

### BMP and RA Commit PGC to Female Fate

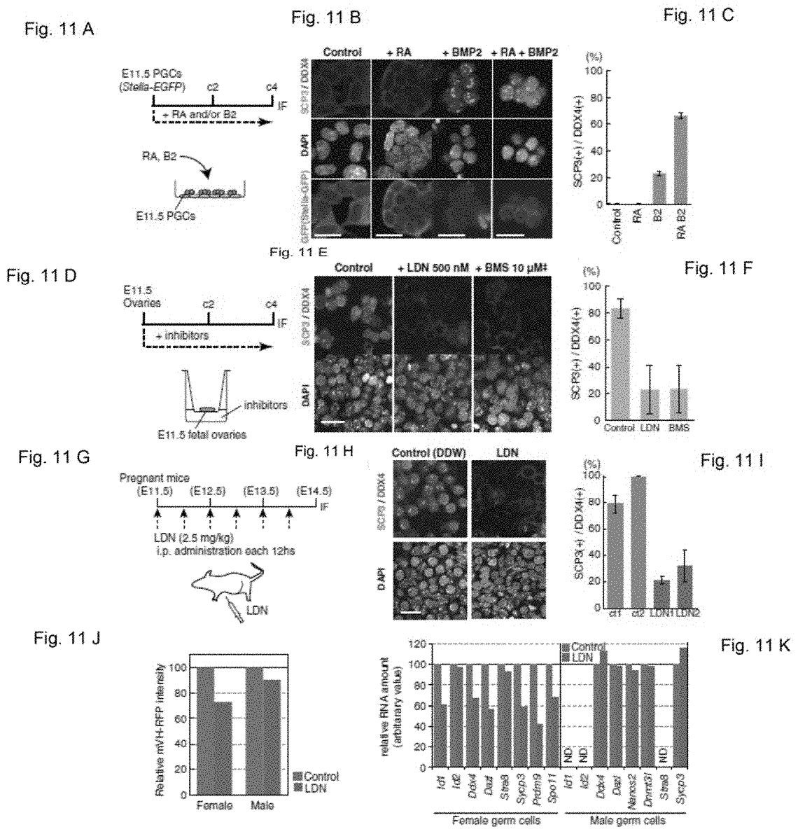

The present inventors next examined the role of RA and BMP signaling in female sex determination in PGC. The present inventors isolated PGC at E11.5 from Stella-EGFP (SG) transgenic embryo (Payer et al., 2006; Seki et al., 2007) by FACS and cultured same with RA, BMP2, or both for 4 days (FIG. 11A). Neither control culture nor culture with RA alone induced SCP3 and did not significantly affect SG and DDX4 expression, but culture with RA and BMP2 induced a substantial increase (˜65.6%) in SCP3-positive (+)/DDX4-strongly positive (+)/SG-negative (−) induction (FIGS. 11B and C). Cultures with BMP2 alone also induced SCP3-positive (+)/DDX4-strongly positive (+)/SG-negative (−) cells (˜22.9%) (FIGS. 11B and C).

The present inventors performed culture of whole embryonic ovaries at E11.5 to investigate the effects of RA or inhibitors of BMP signal transduction on the induction of germ cell fate in female. The results showed that both types of inhibitors inhibited the progression to germ cell fate in female, as shown by the expression of SCP3 (FIG. 11D-F). Similarly, inhibition of BMP signaling under in vivo conditions by administration of LDN193189 to pregnant female every 12 hr from E11.5 to E14.0 caused a significant impairment of progression to female germ cell fate at E14.5 (FIG. 11G-I). The present inventors isolated female or male VR(+) cells from LDN-treated embryos by FACS at E14.5 and examined the expression of important genes of female or male germplasm development by quantitative (q)PCR. This analysis showed that LDN-administered female cells up-regulated key genes in late PGC/female germplasm development, such as Ddx4, Daz1, Sycp3, Prdm9, and Spo11, but interestingly, emerged male germplasm development was unaffected (FIGS. 11J and K). The present inventors conclude that both PGC and PGCLC require RA and BMP signal transduction to acquire female germ cell fate.

### Transcriptional Dynamics of PGCLC/PGC in Determination of Female Sex

Next, the present inventors analyzed the kinetics of transcription during sex determination in PGC/PGCLC females. For this purpose, the present inventors isolated total RNA from PGC [Stella-EGFP(+) cells at E9.5, E10.5, E11.5, E12.5 (female) and E12.5 (male)] (Kagiwada et al, 2013), fetal primary oocytes [Stella-EGFP(+) at E13.5, DDX4-RFP(+) at E14.5 and E15.5], PSG [Stella-EGFP(+) at E13.5 (Kagiwada et al, 2013), DDX4-RFP(+) at E14.5 and E15.5], PGCLCs cultured under control conditions (d4/c0, c3, c5, c7 and c9), PGCLCs cultured with RA since c3 (c5 RB2, c7 RA2 and c9 RA2), PGCLCs cultured with RA since c3 (c5 RAB2, c7 RAB2 and c9 RAB2) and BMP2, and analyzed its transcriptome by RNA sequencing (RNA-seq) (Nakamura et al., 2015).

The Unsupervised Hierarchical Clustering (UHC) revealed that sex undifferentiated PGCs up to E11.5 clustered close to d4 PGCLCs, followed by c3-c9 PGCLCs, and RA or RAB2-stimulated PGCLCs at c5 (c5 RA, c5 RAB2) (FIG. 12A). Fetal primary oocytes (E14.5 and E15.5) and PSG (E14.5 and E15.5) formed distinct clusters, respectively, and surprisingly, RAB2-stimulated PGCLCs at c9 (R9c9) formed robust clusters with fetal primary oocytes (FIG. 12A). Germ cells that started sexual differentiation (male and female germ cells at E12.5 and E13.5) and RAB2-stimulated PGCLCs at c7 (c7 RAB2) and RA-stimulated PGCLCs at c7/c9 (c7/c9RA) formed distinct clusters showing the properties between sex undifferentiated PGC/PGCLCs and fetal primary oocytes/c9 RAB2 cells (FIG. 12A). Consistently, principal component analysis (PGA) revealed a parallel progression of PGCLC that was clustered in close proximity with sex undifferentiated PGC and d4/c3-c9 PGCLC, highlighted the progression along the development of female and male germ cell characteristics, and was cultured with RAB2 along the female pathway, and c9 RAB2 cells clustered with fetal primary oocytes at E14.5/E15.5 (FIG. 12B). In contrast, PGCLCs cultured in RA only partially acquired the characteristics of fetal oocytes (FIG. 12B). Thus, cultured PGCLCs stimulated with RA and BMP2 repeat the progression of the transcriptome of the female pathway to form fetal primary oocytes.

To facilitate the understanding of the dynamics of gene expression during germ cell/PGCLC sex differentiation, the present inventors defined a set of four classes of genes, early PGC genes (318 genes), late germ cell genes (254 genes), fetal oocyte genes (476 genes) and PSG genes (323 genes), that characterize the developmental stages of these cells (FIG. 12C). Early PGC genes increased genes with functional terms of gene ontology (GO), such as “negative regulation of cell differentiation/regulation of the cell cycle” (Prdm1, Prdm14, Tfap2c, Nanog, Sox2, etc.); late germ cell genes increased genes of “sex reproduction/germination” (Daz1, Ddx4, Piwi12, Mae1, Mov1011, etc.); fetal oocyte genes increased genes of “meiosis/female gametogenesis” (Stra8, Rec8, Sycp3, Dmc1, Sycp1, etc.); and PSG genes increased genes of “piRNA metabolic processes/male gametogenesis” (Nanos2, Dnmt31, Tdrd9, Tdrd5, Piwi11, etc.) (FIG. 12C).

As shown in FIGS. 12C and D, PGCLCs cultured with RAB2 gradually acquired late germ cell and fetal oocyte genes, whereas down-regulated early PGC genes. In contrast, PGCLCs cultured with RA only partially showed such progression (FIGS. 12C and D): for example, c9 RAB2 cells up-regulated key genes for meiosis (Stra8, Rec8, Sycp3, Spycp1, Spo11, Dmc1, Hormad1, and Prdm9) (all in fetal oocytes) and oogenesis (Fig1a, Ybx2, Nobox, and Cpeb1) to levels similar to those of E14.5/E15.5 fetal oocytes (FIG. 12E). In contrast, c9 RA cells did not show sufficient acquisition of such genes, despite up-regulation of the StraB and RecB genes in response to RA events in a heterogeneous cell situation (Oulad-Abdelghani et al, 1996; Mahony et al, 2011) (FIG. 12E). Consistent with the role of BMP signaling in determining the fate of female germ cells, PGCLCs cultured with RAB2 and developing female germ cells expressed receptors and important targets of BMP signaling in a similar fashion.

The present inventors identified a gene that was up-regulated in c9 RA cells compared to c9 RAB2 cells (323 gene: RA gene). Such gene was also up-regulated compared to fetal primary oocytes at E14.5/E15.5 and enriched for “cell adhesion/vascular development/embryonic organogenesis” (Hoxa5, Hesx1, Pax6, Lmx1b, Pitx2, Dnmt3b, etc.). Thus, BMP signaling is important not only to strongly drive the female pathway, but also to suppress RA-induced inappropriate developmental programs.

### Role of STRA8 in Development of Fetal Primary Oocytes

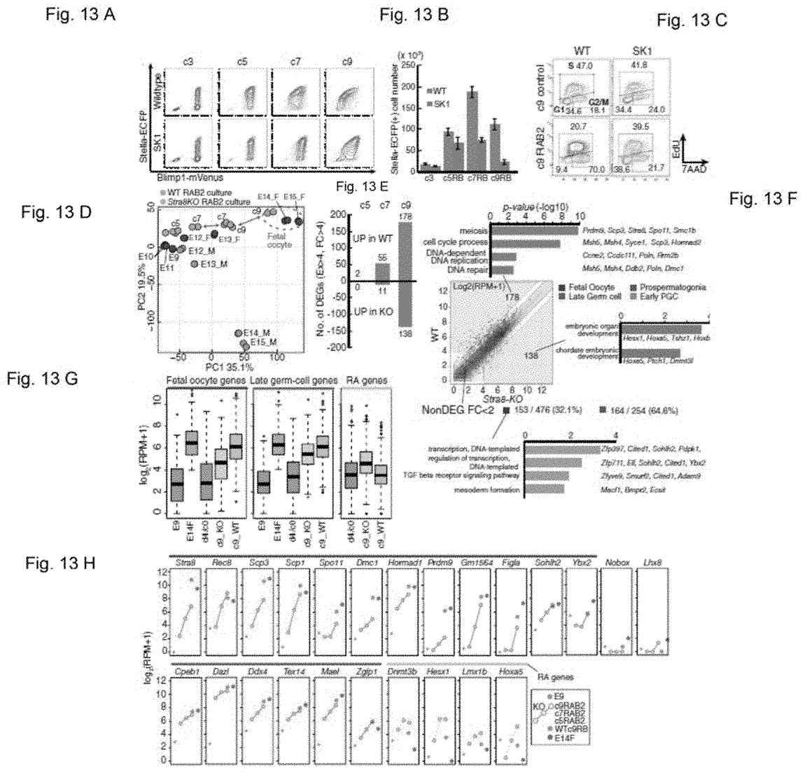

The present inventors next evaluated the effects of loss of StraB during fetal primary oocyte differentiation from PGCLC. The present inventors generated Stra8-Knockout BVSC ESCs (XY) of several strains using the CRISPR/Cas9 system (Ran et al, 2013a, b) and confirmed the frameshift deletion and loss of STRA8 expression in the target exon in these strains. Since the three independent strains (StraB-knockout (SK) 1, 2, 3) showed essentially identical phenotype, the results are presented using the representative strain SK1. Different from wild-type PGCLC, SK1 cells cultured with RAB2 continued to retain robust BVSC expression until c7 and only showed mild down-regulation of BVSC at c9 (FIG. 13A). Compared to wild-type cells, SK1 cells did not proliferate as effectively in response to RAB2, but continued to show a cyclic profile at c9 (FIGS. 13B and C). This indicates that they were not able to proceed to the meiosis period. This finding is in good agreement with the fact that StraB knockout germ cells do not undergo DNA replication before meiosis and are subsequently eliminated (Baltus et al, 2006; Dokshin et al, 2013).

The present inventors determined the transcriptome of SK1 cells. PGA revealed that SK1 RAB2 cells progressed along the female pathway for a long time and acquired at c9 the properties similar to those of wild-type c7 cells which are closer to fetal primary oocytes at E13.5 (FIG. 13D). Compared with wild-type cells, SK1 cells exhibited numerous differently expressed genes (DEGs) since c7 (FIG. 13E), and genes (178 genes) that were not completely up-regulated in c9 SK1 cells were enriched in those (e.g., Prdm9, Spycp3, Spo11, Smc1b, Msh4, Msh5, Dmc1, Ccdc111, and Po1n) for “meiotic/cell cycle processes” (FIG. 13F), and all 12 genes reported to be Stra8-dependent (Soh et al., 2015) were down-regulated in c9 SK1 cells.

However, it is noteworthy that c9 SK1 cells up-regulated 32.1% of fetal oocyte genes (153/476 genes) in a relatively normal manner, including those associated with oocyte development, such as Ybx2 and Soh1h2 (FIG. 13F-H). Interestingly, the genes abnormally up-regulated in c9 SK1 cells were enriched in “embryonic organ development/fetal embryogenesis” and overlapped with RA genes abnormally up-regulated in c9 RA cells (FIGS. 13F and G). In contrast, c9 SK1 cells acquired late germ cell genes in a relatively normal fashion [164/254 genes (64.6%)] (FIG. 13F-H). Thus, STRA8, in cooperation with the effector(s) of BMP signaling, ensures adequate expression levels of several genes involved in meiosis, in addition to repressing the unnecessary developmental pathways induced by RA.

### Cell Competence to Induce Female Fate in Response to RA and BMP

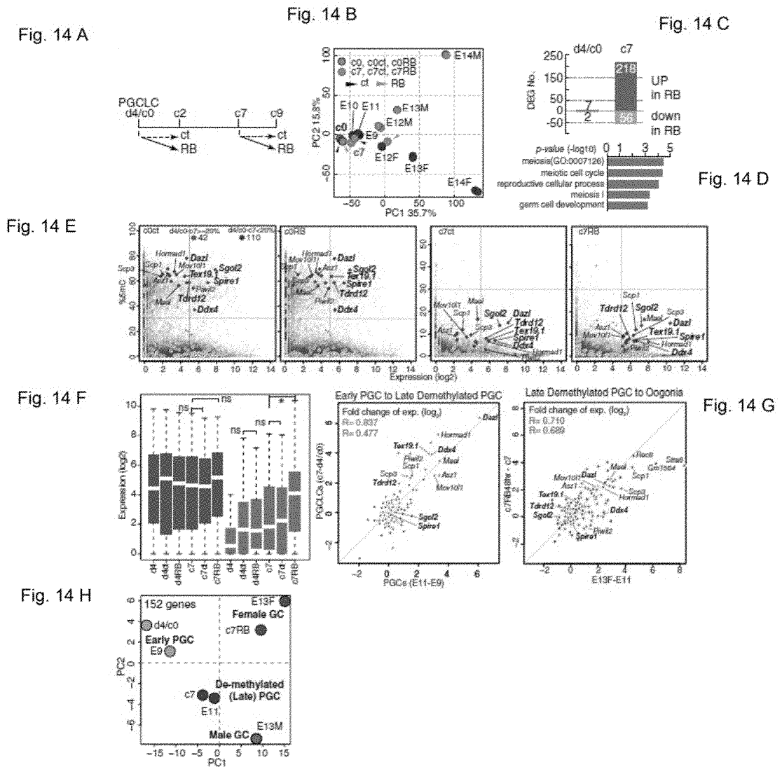

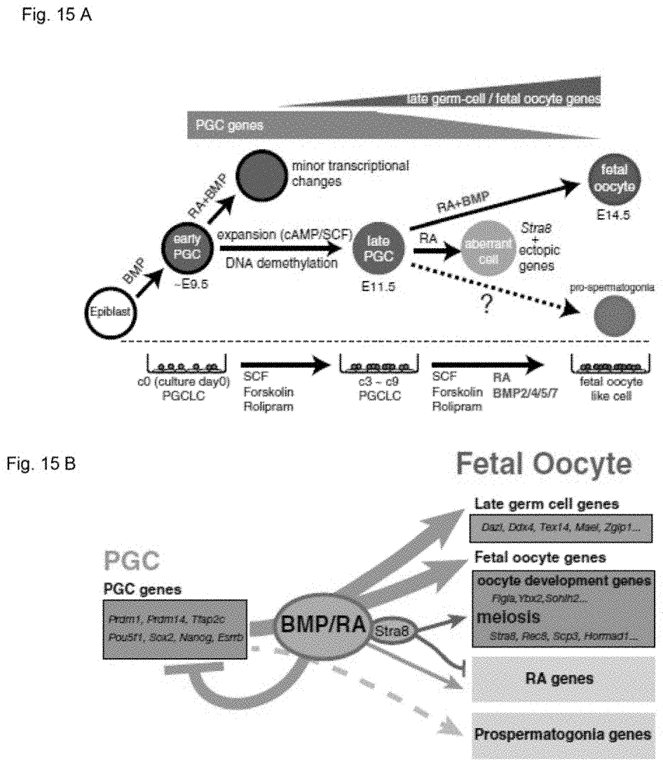

Signaling of osteogenic proteins determines the fate of ectoderm/EpiLCs to PGC/PGCLC, but does not directly induce them to the fate of female (Hayashi et al, 2011). Therefore, the present inventors attempted to clarify the status of cells that result in female fate in response to RA and BMP signaling. The present inventors cultured PGCLCs of d4/c0 or c7 with RA and BMP2 for 2 days and evaluated their responses by examining their transcriptases (FIGS. 14A and B). Compared to the control, c7 PGCLC with RAB2 showed a substantial number of DEGs (218 up-regulated genes and 56 down-regulated genes) and proceeded along the female pathway, up-regulating a group of genes enriched in those for meiosis (FIGS. 14C and D). In contrast, d4/c0 PGCLCs with RAB2 showed only small changes in gene expression (up-regulation: 7 genes; down-regulation; 2 genes) and did not progress toward a female fate (FIGS. 14B and C).

The present inventors reasoned that DNA demethylation of important genes for meiosis during amplification culture may be the basis for PGCLC acquisition of competence to respond to RA and BMP2. The present inventors analyzed the relationship between the level of 5-methylcytosine (5mC) in the promoter and the expression level of a gene classified as being involved in “meiosis” [GO term: meiosis GO: 0007126]. Of such 152 genes, 42 genes, including Stra8, Spo11, Sycp3, Dpep3, Daz1, Ddx4 and Piwi12, showed >20% promoter demethylation between d4/c0 and c7. In contrast, 110 genes did not show significant changes in the promoter 5mC level during culture (<20%) and consisted of those involved in general processes such as “DNA repair” and “response to DNA damage stimuli” (e.g., M1h1, Brca2, Fanca, Cdc20, Plk1) (FIG. 14E). In d4/c0 PGCLC, 42 genes showed high promoter 5mC levels and no/low expression, while 110 genes were mostly unmethylated and showed various expression levels. The distribution thereof was similar to the distribution of all genes with low promoter 5mC levels (FIGS. 14E and F). In c7 PGCLC, 42 genes were demethylated like almost all other genes, e.g., Daz1 and Hormad1, some of the 42 genes were partially up-regulated, but 110 genes remained unmethylated and maintained an expression level distribution similar to the distribution of expression levels in d4/c0 PGCLC (FIGS. 14E and F). Notably, at c7, 42 genes showed specific and robust activation in response to RA and BMP2, whereas 110 genes showed only mild up-regulation (FIGS. 14E and F). Taken together, these findings indicate that the progression of promoter DNA demethylation of related genes during PGCLC expansion induces a basal activation or tolerance state for activation of such genes and, consequently, acquires a fully activated state in response to RA and BMP signaling, and forms the fate of female germ cells.

To test the relevance of this concept to germplasm development in vivo, the present inventors compared the difference in the expression of 152 genes in PGCs between E9.5 and E11.5 with the difference in the expression of these genes i in PGCLCs between d4/c0 and c7; the present inventors then compared the differential expression of 152 genes between E11.5 PGCs and E13.5 fetal primary oocytes, and the differential expression between c7 PGCLCs and c7 PGCLC stimulated for 2 days by BMP2 and RA. As shown in FIG. 14G, the difference (up-regulation) in the expression of 42 genes during successive stages of germ cell and PGCLC development was highly correlated; PCA using 152 genes consistently clustered d4/c0 PGCLC in close proximity to E9.5 PGC, c7 PGC in close proximity to E11.5 PGC, and c7 PGCLC cultured for 48 h with RAB2 in close proximity to E13.5 female germplasm (FIG. 14H). The present inventors conclude that the PGCLC-based in vitro system accurately reproduces the mechanism for the acquisition of female germ cell fate in vivo.

[III] Expansion of PGC/PGCLC by Cyclosporine A and Combination of the Drug with PDE4 Inhibitor and Forskolin

### <Materials and Methods>

The mouse and feeder cell described in Example [I] were used. Purification of PGC/PGCLC, immunostaining, transplantation of PGCLC into mouse testes and analysis thereof, as well as RNA-seq and analysis thereof, were performed in the same way as in Example [I], from the generation of ESC to the induction of differentiation into PGCLC.

Expansion culture of PGC/PGCLC to investigate the combined effect of cyclosporin A (CsA) was performed as in Example [I], except that 5 μM CsA was added to the medium in addition to 10 μM forskolin and 10 μM loripram (FR10).

In the experiment to investigate the promoting effect of CsA alone on the expansion of PGCLCs and the mechanism of the promoting effect, PGCLC was maintained and cultured in the same way as in Example [I] except for the addition of various concentrations (10, 5, 1, and 0 μM) of CsA or FK506 (tacrolimus) to the medium in place of FR10.

### Cell Cycle Analysis

The analysis was performed as in Example [I] except that EdU (10 μM) was used in place of BrdU, and Click-iT™ Plus EdU Alexa Fluor™ 647 Flow Cytometry Assay Kit (Thermo Fisher Scientific) was used for detection of EdU uptake.

### Detection of Apoptotic Cell

d4c7 PGCLC cultured in FR10 or FR10+CsA was dispersed in single cells by TrypLE treatment, and stained using Annexin V Apoptosis Detection Kit APC (eBioscience) according to the manufacturer's instructions. The stained samples were analyzed using BD FACSAriaIII (BD) with FACSDiva (BD) software, and PGCLC was identified by BV fluorescence. Three biological replicates were analyzed for each sample.

### Intracytoplasmic Sperm Injection (ICSI)

Spermatozoons were collected from the mouse testis that received transplantation of PGCLC by the method described in Example [I], and directly injected with a micromanipulator into the ovum collected from BDF1 females that were superovulated by injecting PMSG and hCG. The obtained 2-cell embryos were transferred to the fallopian tube of pseudopregnant ICR females 0.5 days after pregnancy (dpc). Pups were delivered by cesarean section at 18.5 dpc.

### <Results>

### Effect of CsA on the Culture System of PGCLC

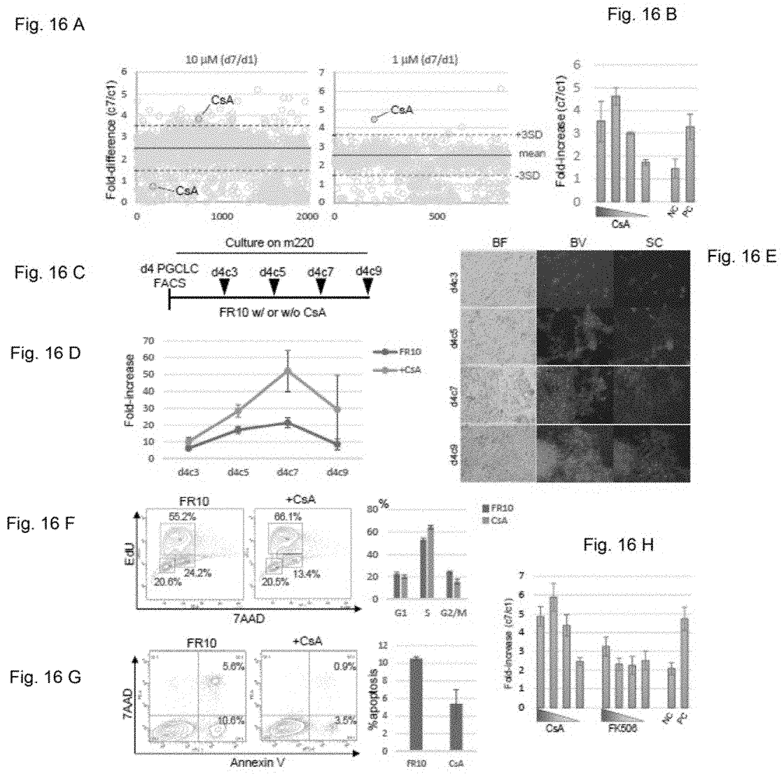

To find compounds that support PGCLC proliferation in addition to forskolin and PDE4 inhibitors, the results of the compound library screening performed in Example [I] were analyzed in detail. As a result, three CsAs were contained in the screening data, and two of which exceeded +3SD (FIG. 16A). To confirm the effect of CsA, different concentrations of CsA were applied to PGCLCs, and 5 μM of CsA was found to be the most optimal concentration for growing PGCLCs (FIG. 16B). Next, whether CsA could further support PGCLC proliferation in the presence of forskolin and PDE4 inhibitor (FR10) was examined (FIG. 16C), and it was found that the addition of CsA could further amplify PGCLCs by about 50-fold on average (FIG. 16D). The PGCLCs amplified by CsA formed flat colonies and strongly expressed BVSCs (FIG. 16E).

To investigate the effect of CsA on the culture system of PGCLCs, cell cycle analysis (FIG. 16F) and detection of apoptotic cells (FIG. 16G) were performed. The results showed that PGCLCs treated with CsA had an increased percentage of S-stage (FIG. 16F, right) and a decreased percentage of apoptotic cells (FIG. 16G, right) compared to PGCLCs treated with FR10 alone. These results suggest that CsA supports PGCLC proliferation by promoting the cell cycle of PGCLCs and inhibiting apoptosis.

CsA is a known immunosuppressive compound, and it is also known to act on mitochondria to suppress apoptosis. To investigate which causes the PGCLC proliferation effect of CsA, influence of FK506 on PGCLC was analyzed. FK506 is a compound known to exert immunosuppressive effects by action mechanism similar to that of CsA, but with no effect on mitochondria. The results showed that FK506 did not have a proliferative effect on PGCLCs (FIG. 16H). These results suggest that CsA acts on the mitochondria of PGCLCs and supports their proliferation thereof by inhibiting apoptosis.

### Transcriptional and Epigenetic Properties of PGCLCs in Amplified Culture by CsA and Effect of CsA on In Vivo PGC

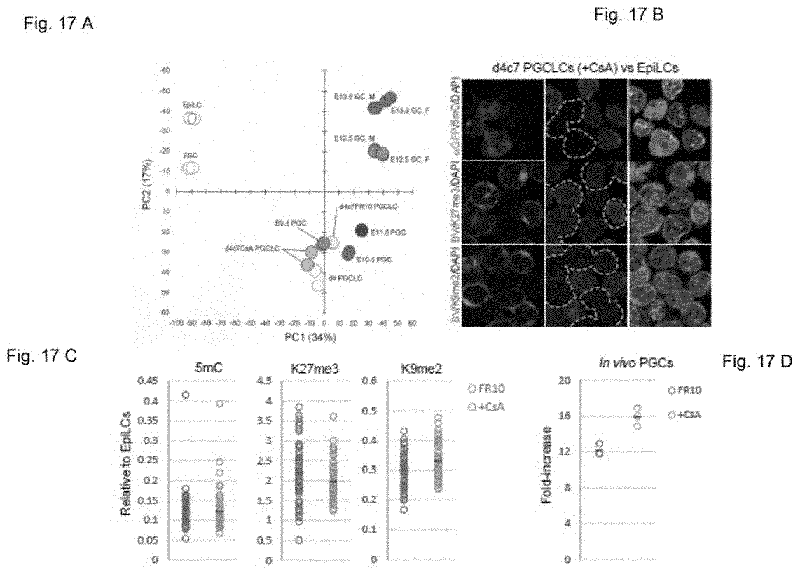

Next, the detailed transcriptional properties of PGCLCs in amplification culture with CsA were determined using the same method as in Example [I] (FIG. 4B) (FIG. 17A). Principal component analysis (PGA) revealed that PGCLC amplified by adding CsA to FR10 showed a similar gene expression pattern to PGCLC amplified by FR10 (FIG. 17A). The epigenetic properties were also analyzed by IF, and it was found that PGCLC amplified by adding CsA to FR10 had similar epigenetic properties to those amplified by FR10 (FIGS. 17B and C). These results suggest that the PGCLC amplified by adding CsA to FR10 have similar properties to PGCLC amplified by FR10.

To test whether CsA can support in vivo PGC growth, PGC of E9.5 was recovered and cultured in vitro. The results showed that the addition of CsA to FR10 could amplify the in vivo PGC to about 16-fold (FIG. 17D). These results indicate that CsA can support not only PGCLCs but also expansion of PGCs in vivo.

### Spermatogenesis Ability of PGCLCs Amplified by CsA

Next, whether PGCLCs amplified by FR10+CsA would retain their function as PGCLCs was assessed. For this purpose, d4c7 PGCLCs amplified with FR10+CsA were transplanted into the testes of neonatal W/W^{v} mice lacking endogenous germ cells. Analysis of the testes 10 weeks after implantation showed that they contained numerous deferents with evidence of spermatogenesis and indeed produced abundant sperm (FIG. 18A-E). Furthermore, when intracytoplasmic sperm injection (ICSI) was performed using the obtained sperm, normal pups were obtained (FIGS. 18F-I) and showed normal growth (FIG. 18J). These results indicate that PGCLCs amplified with CsA are capable of differentiating into functional spermatozoa.

### INDUSTRIAL APPLICABILITY

According to the present invention, there is a possibility that ovum can be produced in vitro from PGC/PGCLC. Therefore, the present invention is expected to be applied to basic research relating to infertility and assisted reproductive medicine, and is extremely useful.

This application is based on a patent application No. 2017-231294 filed in Japan (filing date: Nov. 30, 2017), the contents of which are incorporated in full herein by reference.

1. A method for expanding a primordial germ cell (PGC) or isolated pluripotent stem cell-derived primordial germ cell-like cell (PGCLC), comprising culturing PGC or PGCLC in the presence of a phosphodiesterase 4 (PDE4) inhibitor and/or cyclosporine A.

2. The method according to claim 1, wherein the PGC or PGCLC is cultured under a condition further comprising forskolin.

3. A reagent for expanding PGC or PGCLC, comprising a PDE4 inhibitor and cyclosporine A.

4. The reagent according to claim 3 comprising forskolin in combination.

5. A method for inducing oocyte from PGC or PGCLC, comprising culturing PGC or PGCLC in the presence of a bone morphogenetic protein (BMP) and retinoic acid (RA).

6. The method according to claim 5, wherein the BMP is one or more selected from BMP2, BMP5 and BMP7.

7-8. (canceled)

9. A method for inducing oocyte from PGC or PGCLC, comprising
(a) expanding PGC or PGCLC by the method according to claim 1, and
(b) culturing the PGC or PGCLC obtained in step (a) in the presence of BMP and RA culturing in the presence of BMP and RA.


SEQUENCE LISTING (46 sequences)

SEQ ID NO: 1 - DNA, 22 bp, Artificial Sequence
  Synthetic oligonucleotide primer sequence
tggatggtgtggccaatgcyct

SEQ ID NO: 2 - DNA, 28 bp, Artificial Sequence
  Synthetic oligonucleotide primer sequence
ccacctgcacgttgcccttkgtgcccag

SEQ ID NO: 3 - DNA, 30 bp, Artificial Sequence
  Synthetic oligonucleotide primer sequence
agaatctggtccacaggaaaagggcctgat

SEQ ID NO: 4 - DNA, 30 bp, Artificial Sequence
  Synthetic oligonucleotide primer sequence
aggcttagcccctgagctgcttgttaactg

SEQ ID NO: 5 - DNA, 25 bp, Artificial Sequence
  Synthetic oligonucleotide primer sequence
aatagaccctaaccagttggtgcat

SEQ ID NO: 6 - DNA, 28 bp, Artificial Sequence
  Synthetic oligonucleotide primer sequence
aagagtaagtggaatccatgttgaagga

SEQ ID NO: 7 - DNA, 30 bp, Artificial Sequence
  Synthetic oligonucleotide primer sequence
ccggatgaatgtcagctactgggctatctg

SEQ ID NO: 8 - DNA, 30 bp, Artificial Sequence
  Synthetic oligonucleotide primer sequence
ttcggggcgaaaactctcaaggatcttacc

SEQ ID NO: 9 - DNA, 25 bp, Artificial Sequence
  Synthetic oligonucleotide primer sequence
caccggagatggcggcagagacaat

SEQ ID NO: 10 - DNA, 25 bp, Artificial Sequence
  Synthetic oligonucleotide primer sequence
aaacattgtctctgccgccatctcc

SEQ ID NO: 11 - DNA, 25 bp, Artificial Sequence
  Synthetic oligonucleotide primer sequence
caccgcctgtggcagactctctctg

SEQ ID NO: 12 - DNA, 23 bp, Artificial Sequence
  Synthetic oligonucleotide primer sequence
aaaccagagagagtgccacaggc

SEQ ID NO: 13 - DNA, 21 bp, Artificial Sequence
  Synthetic oligonucleotide primer sequence
taaggccaggggaaggcagac

SEQ ID NO: 14 - DNA, 22 bp, Artificial Sequence
  Synthetic oligonucleotide primer sequence
cgaagggccatctcacagggtc

SEQ ID NO: 15 - DNA, 30 bp, Artificial Sequence
  Synthetic oligonucleotide primer sequence
ggaggcaaagacaggatcctaagcaaacta

SEQ ID NO: 16 - DNA, 30 bp, Artificial Sequence
  Synthetic oligonucleotide primer sequence
tccctagtgctctataaaattgaggtaatt

SEQ ID NO: 17 - DNA, 20 bp, Artificial Sequence
  Synthetic oligonucleotide primer sequence
ccgccgacatccccgattac

SEQ ID NO: 18 - DNA, 20 bp, Artificial Sequence
  Synthetic oligonucleotide primer sequence
gcagttgcacgggcttcttg

SEQ ID NO: 19 - DNA, 26 bp, Artificial Sequence
  Synthetic oligonucleotide primer sequence
accagaaaactaagaagtgtcctgaa

SEQ ID NO: 20 - DNA, 26 bp, Artificial Sequence
  Synthetic oligonucleotide primer sequence
agacaggacagtaagtcagtgatgct

SEQ ID NO: 21 - DNA, 20 bp, Artificial Sequence
  Synthetic oligonucleotide primer sequence
caacagagcctcaccctctc

SEQ ID NO: 22 - DNA, 20 bp, Artificial Sequence
  Synthetic oligonucleotide primer sequence
agaaatccgagaagcacgaa

SEQ ID NO: 23 - DNA, 22 bp, Artificial Sequence
  Synthetic oligonucleotide primer sequence
cacaaaggtggagcgtgaatac

SEQ ID NO: 24 - DNA, 22 bp, Artificial Sequence
  Synthetic oligonucleotide primer sequence
gcattcagtaggctcgtgtcaa

SEQ ID NO: 25 - DNA, 21 bp, Artificial Sequence
  Synthetic oligonucleotide primer sequence
cagcttcagtagcagcacaag

SEQ ID NO: 26 - DNA, 21 bp, Artificial Sequence
  Synthetic oligonucleotide primer sequence
catgactcgtcatcaactgga

SEQ ID NO: 27 - DNA, 22 bp, Artificial Sequence
  Synthetic oligonucleotide primer sequence
gatggacatgagatcattggac

SEQ ID NO: 28 - DNA, 21 bp, Artificial Sequence
  Synthetic oligonucleotide primer sequence
ataccagggagcaatcctgac

SEQ ID NO: 29 - DNA, 22 bp, Artificial Sequence
  Synthetic oligonucleotide primer sequence
gccggagaaggaggagattaaa

SEQ ID NO: 30 - DNA, 22 bp, Artificial Sequence
  Synthetic oligonucleotide primer sequence
agcagcctttctcaatgagtct

SEQ ID NO: 31 - DNA, 19 bp, Artificial Sequence
  Synthetic oligonucleotide primer sequence
gtgttgcagcagtgggaac

SEQ ID NO: 32 - DNA, 21 bp, Artificial Sequence
  Synthetic oligonucleotide primer sequence
gctttcattctctggctctga

SEQ ID NO: 33 - DNA, 23 bp, Artificial Sequence
  Synthetic oligonucleotide primer sequence
cctggctacaaattctcattttc

SEQ ID NO: 34 - DNA, 25 bp, Artificial Sequence
  Synthetic oligonucleotide primer sequence
tgtttttgtttgttttgttttggga

SEQ ID NO: 35 - DNA, 22 bp, Artificial Sequence
  Synthetic oligonucleotide primer sequence
agcatgaagtgtctcactagca

SEQ ID NO: 36 - DNA, 22 bp, Artificial Sequence
  Synthetic oligonucleotide primer sequence
cattaacagggcaaggcaccta

SEQ ID NO: 37 - DNA, 22 bp, Artificial Sequence
  Synthetic oligonucleotide primer sequence
agagaagaatgccagttgggtt

SEQ ID NO: 38 - DNA, 22 bp, Artificial Sequence
  Synthetic oligonucleotide primer sequence
acaacgctttattcagcagcag

SEQ ID NO: 39 - DNA, 20 bp, Artificial Sequence
  Synthetic oligonucleotide primer sequence
tcgggtttctctcctgtttg

SEQ ID NO: 40 - DNA, 20 bp, Artificial Sequence
  Synthetic oligonucleotide primer sequence
gttatcccaccgggaacttg

SEQ ID NO: 41 - DNA, 23 bp, Artificial Sequence
  Synthetic oligonucleotide primer sequence
ttacccatcaaaccattccttct

SEQ ID NO: 42 - DNA, 24 bp, Artificial Sequence
  Synthetic oligonucleotide primer sequence
aacccaaagaacttcagtgagagc

SEQ ID NO: 43 - DNA, 22 bp, Artificial Sequence
  Synthetic oligonucleotide primer sequence
caaagctgaagcaaaggaagag

SEQ ID NO: 44 - DNA, 23 bp, Artificial Sequence
  Synthetic oligonucleotide primer sequence
aattaagcaggctgacttggttg

SEQ ID NO: 45 - DNA, 21 bp, Artificial Sequence
  Synthetic oligonucleotide primer sequence
gatgctgtgagccaaggcaag

SEQ ID NO: 46 - DNA, 22 bp, Artificial Sequence
  Synthetic oligonucleotide primer sequence
ggctcctgatcaacagcatcac